(19) United States
(12) Patent Application Publication
(10) Pub. No.: US 2026/0357242 A1
(43) Pub. Date: Nov. 12, 2026

(54) METHODS AND SYSTEMS FOR ULTRA-HIGH-THROUGHPUT SCREENING OF YEAST SECRETIONS USING MOLECULAR SENSORS ANCHORED ON MOTHER YEAST CELLS

(71) Applicant: City University of Hong Kong, Hong Kong (HK)

(72) Inventors: Chia-Hung CHEN, Hong Kong (HK); Wenxin JIANG, Hong Kong (HK)

(21) Appl. No.: 19/666,395

(22) Filed: May 4, 2026

Related U.S. Application Data
(60) Provisional application No. 63/798,616, filed on May 2, 2025.

(51) Int. Cl. C12N 15/10; C07K 14/57; C07K 14/765; C12N 1/16; C12N 15/115; C12R 1/84; C12R 1/865; G01N 21/64; G01N 33/554; G01N 33/68

(52) U.S. Cl. CPC C12N 15/1037; C07K 14/57; C07K 14/765; C12N 1/16; C12N 15/1058; C12N 15/115; G01N 21/6428; G01N 33/554; G01N 33/6866; C12N 2310/16; C12R 2001/84; C12R 2001/865; G01N 2021/6439; G01N 2333/57; G01N 2333/765

(57) ABSTRACT

A method for ultra-high-throughput screening of yeast metabolism is provided. The method includes providing a population of yeast cells and anchoring a plurality of molecular sensors on membrane surfaces of mother yeast cells to form molecular sensors on membrane surfaces of mother cells (MOMS). The molecular sensors are selectively retained on the mother yeast cells during budding and are not transferred to daughter cells. The yeast cells are incubated under conditions that allow secretion of metabolic products from individual yeast cells. Secreted metabolic products are captured by the molecular sensors anchored on the membrane surface of the mother yeast cells. Interaction between the molecular sensors and the captured metabolic products generates detectable signals, which are used to screen the yeast cells and identify cells exhibiting desired metabolic secretion phenotypes.

### REFERENCE TO SEQUENCE DISCLOSURE

The sequence listing file under the file name “P3540US01_Sequence Listing.xml” submitted in ST.26 XML file format with a file size of 287,821 bytes created on May 4, 2026 and filed on May 4, 2026 is incorporated herein by reference.

### CROSS-REFERENCE TO RELATED APPLICATIONS

The present application claims priority from U.S. Provisional Utility Patent application no. 63/798,616 filed May 2, 2025; the disclosure of which is incorporated herein by reference in its entirety.

### FIELD OF THE INVENTION

The present invention relates to microbial screening technologies, and particularly to ultra-high-throughput single-cell screening of yeast metabolism using membrane-anchored molecular sensors.

### BACKGROUND OF THE INVENTION

Yeasts play a crucial role in many aspects of modern biotechnology, producing essential metabolites for pharmaceuticals, the food industry, environmental applications, and broader life science and biomedical fields. Extracellular secretions from yeast are particularly important for key metabolic processes such as fermentation, chemical decomposition, antimicrobial activity, and drug development. Directed evolution of yeast strains relies on identifying superior variants from large mutant libraries for improved chemical production and pharmaceutical applications. This process requires practical methods for analyzing extracellular secretions at the single-cell level across extremely large variant libraries (10⁶-10⁷ mutants) in order to identify rare, high-performing secretory strains. Although emerging molecular sensing technologies provide promising approaches for detecting intracellular compounds and extracellular secretions at the single-cell level, existing methods still struggle to achieve an optimal balance among sensitivity, throughput, and processing speed. These limitations present significant challenges for large-scale analysis and screening of yeast metabolic secretions.

Immunosorbent assays provide a promising strategy for measuring extracellular secretions from yeast by capturing target molecules for labeling and subsequent analysis. Enzyme-linked immunosorbent spot (ELISpot) assays, adapted from ELISA, generate visible spots that indicate secretory activity at the cellular level. More recently, microtiter plates containing nanoliter-scale microwells have been developed to enable parallel single-cell assays. In these systems, secreted molecules are captured on pre-coated glass slides functionalized with antibodies or aptamers, allowing multiplexed profiling of secretory products. However, the throughput of these approaches remains limited, typically analyzing only about 10³-10⁴ cells per assay. Mass spectrometry techniques, such as gas chromatography-mass spectrometry (GC-MS) and high-performance liquid chromatography-mass spectrometry (HPLC-MS), provide versatile platforms for detecting diverse metabolites secreted by yeast. Nevertheless, these techniques generally operate at extremely low throughput—often analyzing approximately one cell per experiment—making them unsuitable for large-scale single-cell secretion studies.

Fluorescence-activated cell sorting (FACS) provides a high-throughput alternative, capable of analyzing approximately 10³-10⁴ cells per second. However, conventional FACS primarily evaluates cell size, surface markers, and intracellular molecules, which limits its applicability for direct analysis of extracellular secretions. Intracellular staining (ICS) methods have been adapted to indirectly infer extracellular secretion profiles; however, these approaches are often labor-intensive, prone to artifacts, and may compromise cell viability. As a result, such methods are not well suited for large-scale screening and identification of high-secretion yeast strains for metabolic analysis and strain optimization.

Fluorescence-activated droplet sorting (FADS) has emerged as a powerful technique for high-throughput single-cell assays. In FADS systems, microfluidic droplet devices generate monodisperse water-in-oil droplets (~10-40 pL), each encapsulating a single cell together with chemical sensors that produce fluorescence signals upon detecting target metabolites. This configuration enables efficient detection and sorting of individual cells based on their metabolic activity. FADS has been widely applied to screen mutant yeast cells for metabolic engineering applications by measuring both intracellular molecules and extracellular secretions.

Intracellular single-cell molecular assays are typically performed by lysing cells within droplets, which releases intracellular components and triggers fluorescent signals through substrate reactions, thereby enabling the selection of improved enzyme variants. However, this approach prevents recovery of viable cells for downstream functional analysis. Alternatively, intracellular enzyme activities can be evaluated through cell surface display strategies, in which enzymes representing intracellular molecules are expressed on the cell surface. These enzymes interact with substrates within droplets to generate fluorescence signals, allowing sorting for directed evolution. Nevertheless, such approaches require genetic modification to display enzymes on the cell surface.

More recently, FADS has been adapted for direct measurement of extracellular secretions, including enzymes and small-molecule metabolites, through enzyme-coupled reactions within droplets. Despite its potential, several challenges remain in applying FADS for large-scale screening of secretory strains. First, versatility is limited because detection often relies on specific enzymatic reactions, which restricts the range of metabolites that can be measured. Many valuable natural products, such as terpenoids and phenolic compounds, remain difficult to detect using droplet-based enzymatic assays. To date, only a limited number of secretions, including α-amylase, lactate, and ethanol, have been successfully measured using such reactions.

Second, sensitivity remains a challenge. Although droplet-based enzyme reactions can enable highly sensitive detection of certain low-abundance molecules, current assays largely focus on intracellular molecules. For extracellular secretions, while α-amylase has been detected at concentrations as low as approximately 0.1 nM in specific cases, the sensitivity threshold for detecting most metabolic molecules remains limited to approximately 10 μM.

Third, throughput and screening speed remain constrained. The rate of single-cell encapsulation is typically low (<10%), which limits processing speeds to approximately 10-200 cells per second, while droplet screening rates generally range from 10² to 10³ droplets per second. These limitations restrict the practical application of FADS in screening very large mutant libraries.

The emerging RNA-Aptamer-in-Droplet (RAPID) screening technology addresses several limitations of conventional FADS by introducing suspended RNA aptamer sensors that generate fluorescence signals upon binding to target molecules through programmable aptamer sequences. This approach enables flexible and potentially multiplexed detection of extracellular secretions. However, several challenges remain. For example, RAPID systems often suffer from limited sensitivity and instability of RNA aptamers during co-incubation with yeast cells, which can lead to false-positive signals and a relatively high detection limit (approximately 260 μM).

Alternatively, genetically engineered living-cell biosensors have been developed for co-encapsulation with producer cells in droplets to detect secreted metabolites. While these biosensors can provide functional detection capabilities, their scalability is limited by challenges associated with strain co-culture and relatively low detection sensitivity (approximately 70 μM for naringenin), which restricts their usefulness for high-throughput metabolic screening.

More recently, droplet-based graphene oxide (GO) aptasensors have improved detection performance by immobilizing aptamers on GO surfaces. This strategy reduces nonspecific binding, minimizes false-positive signals, and improves detection sensitivity to approximately 10 μM. Despite these improvements, overall screening throughput remains constrained by the low probability of single-cell encapsulation in droplets (approximately 1-10%) and relatively modest processing speeds (around 10 cells per second).

There is a persistent trade-off between sensitivity, throughput, and speed-most current technologies struggle to achieve all three simultaneously in yeast secretion analysis. These challenges significantly restrict the scalability and versatility of current platforms for high-throughput, large-scale screening of yeast variants. Therefore, the present invention addresses this need.

### SUMMARY OF THE INVENTION

It is an objective of the present invention to provide the systems and methods to solve the aforementioned technical problems.

In accordance with a first aspect of the present invention, a method for ultra-high-throughput screening of yeast metabolism is provided. The method includes the following steps: providing a population of yeast cells; anchoring a plurality of molecular sensors on membrane surfaces of mother yeast cells to form molecular sensors on membrane surfaces of mother cells (MOMS), in which the molecular sensors are selectively retained on the mother yeast cells during budding and are not transferred to daughter cells; incubating the yeast cells in conditions allowing secretion of metabolic products from individual cell of the yeast cells; capturing the secreted metabolic products using the molecular sensors anchored on the membrane surfaces of the mother yeast cells; generating detectable signals from the molecular sensors upon interaction with the captured metabolic products; and screening the yeast cells based on the detectable signals to identify yeast cells exhibiting a desired metabolic secretion phenotype. In some embodiments, the secreted metabolic products include target metabolites and proteins.

In accordance with one embodiment, the secreted metabolic products include target metabolites and proteins, and the molecular sensors include nucleic acid aptamer sensors configured to bind to the target metabolites and proteins.

In accordance with another embodiment, the aptamer sensors are immobilized on the membrane surfaces of the mother yeast cells via biotin-streptavidin interaction.

In accordance with yet another embodiment, the molecular sensors are immobilized at a density of at least 10⁶ sensors per yeast cell.

In accordance with yet another embodiment, the molecular sensors generate fluorescence signals upon binding to the secreted metabolic products.

In accordance with yet another embodiment, the method further includes encapsulating individual MOMS-coated yeast cells in microfluidic droplets to capture secreted metabolites within the droplets.

In accordance with yet another embodiment, the method further includes disrupting the droplets after incubation to release the yeast cells for subsequent screening.

In accordance with yet another embodiment, the molecular sensors include structure-switching aptamers that emit fluorescence upon binding to a target molecule.

In accordance with yet another embodiment, the target metabolites include vanillin, ATP, glucose, and zinc ions.

In accordance with yet another embodiment, the target proteins include interferon-gamma (IFN-γ) and glycosylated human serum albumin.

In accordance with yet another embodiment, the yeast cells include Saccharomyces cerevisiae or Pichia pastoris.

In accordance with yet another embodiment, the method further includes generating a mutant yeast library and performing directed evolution by selecting yeast variants exhibiting enhanced secretion phenotypes.

In accordance with yet another embodiment, the method further includes performing multiplex detection of multiple metabolites by anchoring different molecular sensors targeting different molecules on the membrane surfaces of the mother yeast cells.

In accordance with a second aspect of the present invention, functionalized yeast cells for detecting extracellular secretions are provided. The functionalized yeast cells include at least a mother yeast cell having a membrane surface; and one or more molecular sensors immobilized on the membrane surface of the mother yeast cell. The molecular sensors are configured to bind secreted molecules produced by the functionalized yeast cells and generate detectable signals, and the molecular sensors remain localized on the mother yeast cell during budding and are substantially not transferred to any daughter cell.

In accordance with one embodiment, the molecular sensors include aptamer sensors.

In accordance with another embodiment, the molecular sensors are immobilized through biotin-streptavidin binding.

In accordance with yet another embodiment, the molecular sensors include fluorescence-based structure-switching aptamers.

In accordance with a third aspect of the present invention, a system for ultra-high-throughput screening of microbial secretions is provided. The system includes: the aforementioned functionalized yeast cells; a droplet microfluidic device configured to encapsulate individual cell of the functionalized yeast cells in droplets; an incubation module configured to allow secretion and capture of metabolites by the molecular sensors; and a detection and sorting device configured to detect signals generated by the molecular sensors and to isolate yeast cells exhibiting desired secretion phenotypes. Specifically, the system is configured to screen at least 10⁷ yeast cells in one cycle, and the system is configured to sort yeast cells at a rate of at least 10³ cells per second.

In accordance with a fourth aspect of the present invention, a mutant enoyl-CoA hydratase/aldolase (ECH) enzyme including at least one amino acid substitution selected from I90N, Y169C, and N212Q relative to a wild-type ECH enzyme is provided.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the invention are described in more details hereinafter with reference to the drawings, in which:

FIGS. 1A-1D depict molecular sensors on the membrane surface of mother yeast cells for single-yeast secretion assays, in which: FIG. 1A depicts a schematic representation showing that molecular sensors anchored on the membrane surface of mother yeast cells (MOMS) selectively attach to mother yeast cells and are not transmitted to daughter cells during the budding process (scale bar: 0.5 μm); FIG. 1B shows the advantages of selective sensor coating on mother yeast cells, where the MOMS platform provides high-sensitivity detection, high-throughput screening capability, and ultra-high-speed sorting performance, enabling practical analysis of more than 10⁷ yeast cells within approximately 40 minutes; FIG. 1C compares the performance of the MOMS platform with various droplet-based screening methods, showing that the MOMS platform demonstrates more than a 10-fold improvement in extracellular secretion assay sensitivity and more than a 30-fold increase in sorting speed for identifying secretory strains; and FIG. 1D depicts a comparative analysis of multiple analytical technologies for microbial extracellular secretion assays indicating that the MOMS platform provides an optimal balance of sensitivity, screening throughput, and sorting speed for detecting a wide range of metabolic molecules secreted by individual microbial cell;

FIGS. 2A-2C depict that MOMS coating is present on yeast cell surface, in which: FIG. 2A depicts that the yeast cell wall is visualized after the binding of Alexa Fluor 488 labeled Concanavalin A (ConA); FIG. 2B shows that the successful coating of MOMS on single yeast cells is observed by Cy5 fluorescence; and FIG. 2C is a merged image of Alexa Fluor 488 and Cy5 fluorescence, indicating that MOMS coating only happens on the yeast cell surface;

FIGS. 3A-3D depict the cell viability test after yeast cells coated with biotin-VANapt-Cy5 and subsequently treated with fluorescein diacetate, in which: FIG. 3A depicts that MOMS coating is visualized under CLSM using Cy5 fluorescence labeling, confirming successful coating on yeast cells; FIG. 3B shows the FDA live-dead staining for assessing cell viability under CLSM; FIG. 3C depicts a merged image of Cy5 and fluorescein fluorescence; and FIG. 3D depicts the flow cytometry analysis demonstrating 93% yeast cell viability after MOMS coating, confirming its biocompatibility;

FIGS. 4A-4B depict the biocompatibility of MOMS coating, in which: FIG. 4A depicts a cell growth curve test confirming that MOMS coating does not affect yeast cell growth across different strains; and FIG. 4B depicts the vanillin productivity test of WT and VAN-3 yeast strains, with and without MOMS coating, using an established absorbance-based method;

FIGS. 5A-5L depict the sensitive multiplexed assay via dense MOMS coating, in which: FIG. 5A depicts that yeast surface is functionalized using a biotin-streptavidin binding strategy to attach biotin-labeled aptamers, forming molecular sensors on the membrane surface of mother yeast cells; FIG. 5B depicts that the Cy5-labeled MOMS on Alexa Fluor 488-ConA-stained yeast cells is visualized during budding, demonstrating exclusive localization of MOMS on mother cells;

FIG. 5C depicts that the incubation of yeast cells with 0-3 μM MOMS results in varying coating densities, reaching saturation at 1.5 μM; FIG. 5D depicts that around 77% of MOMS remain on mother cells after over 30 generations (3 days) while daughter cells exhibit MOMS-free coatings; FIG. 5E depicts that a high-density MOMS (~1.0×10⁷ per cell) persists on mother cells for 72 hours, despite declining proportions of mother cells during proliferation; FIG. 5F depicts that the densely grafted MOMS enable highly sensitive multiplexed assays on mother cells; FIG. 5G depicts that MOMS outperform current droplet based aptamer sensors by leveraging ~1.4×10⁷ immobilized aptamers per cell, enhancing assay sensitivity by >100-fold and reducing the detection limit from 50 μM to 100 nM; FIG. 5H depicts that the MOMS' high sensitivity allows direct detection of ATP secreted by single yeast cells; FIG. 5I depicts a multiplexed assay achieved by coating yeast cells with different MOMS targeting vanillin, ATP, and glucose; FIG. 5J depicts that the fluorescence intensity increases over time following incubation of MOMS-coated yeast cells with vanillin (1.0 mM), ATP (1.0 mM), glucose (1.5M), and Zn²⁺ (2.0 mM); FIG. 5K depicts the confocal microscopy images, confirming simultaneous triplex aptamer coating on yeast cells for multiplexed detection; and FIG. 5L shows the heatmap validation confirming 100% assay specificity for multiplexed metabolite detection;

FIG. 6 depicts the fluorescence calibration for quantifying aptamer concentration;

FIGS. 7A-7B depict the gating strategies for identifying mother yeast cells in flow cytometry analysis, in which: FIG. 7A depicts an initial gate defined based on FSC-A versus SSC-A signals to select the starting yeast cell population for distinguishing yeast cells from debris; and FIG. 7B depicts a second gate established using negative control samples (yeast cells without fluorescence labeling) to further differentiate mother yeast cells coated with fluorophore labeled MOMS from daughter cells;

FIG. 8 depicts the MOMS coating during yeast budding;

FIGS. 9A-9B depict the budding scar analysis, in which: FIG. 9A shows that the aptamer sensors continue to localize on mother cells and multiple bud scars are observed, confirming sensor retention through successive divisions; and FIG. 9B depicts a quantitative image analysis indicating that approximately 87% of the surface-bound sensors remain stably anchored on mother cells even after multiple rounds of cell division in 24 hours;

FIGS. 10A-10C depict the specificity tests for vanillin, glucose, and ATP detection using MOMS, in which: FIG. 10A shows the vanillin specificity assessed by comparing responses to vanillin, vanillyl alcohol, vanillic acid, and ferulic acid; FIG. 10B depicts the glucose specificity tested against fructose and galactose; and FIG. 10C depicts the ATP specificity confirmed by comparing ATP to GTP, UTP, and CTP;

FIG. 11 depicts the sensitivity comparison between MOMS and free-floating aptamer sensors;

FIGS. 12A-12C depict the sensitive secretion measurement over time, in which: FIG. 12A is a schematic illustration of MOMS-coated yeast cells capturing their own secretions directly on the cell surface; FIG. 12B depicts the time lapse visualization of ATP secretion from single yeast cells captured by MOMS, monitored using CLSM; and FIG. 12C depicts the flow cytometry analysis showing a progressive increase in yeast ATP secretion over time;

FIGS. 13A-13D depict the molecular detection at different concentrations, in which: FIG. 13A depicts the vanillin assay—MOMS-coated yeast cells are used to detect vanillin concentrations ranging from 0 to 1.00 mM; FIG. 13B depicts the vanillin sensing kinetics—Fluorescence intensity is monitored over time (0-5 hours) to evaluate vanillin detection kinetics; FIG. 13C depicts the ATP assay—MOMS-coated yeast cells are used to detect ATP concentrations ranging from 0 to 4.0 mM; and FIG. 13D depicts the glucose assay—MOMS-coated yeast cells detect glucose concentrations between 0 and 2.0 M;

FIGS. 14A-14D depict the combined MOMS coating on yeast cells, in which: FIG. 14A depicts that MOMS containing biotin-ATPapt-Cy5 are coated on yeast cells; FIG. 14B depicts that MOMS containing biotin-VANapt-FITC are coated on yeast cells; FIG. 14C depicts that MOMS containing biotin-GLUapt-Cy3 are coated on yeast cells; and FIG. 14D depicts the merged fluorescence channels confirming the simultaneous coating of different MOMS on the same yeast cells;

FIGS. 15A-15G depict the high-throughput screening, in which: FIG. 15A depicts that MOMS-enabled high-throughput screening involves capturing single yeast cells and screening them via flow cytometry to identify secretory strains; FIG. 15B depicts that MOMS removes droplets before screening, allowing conventional flow cytometry (10³-10⁴ Hz) despite low droplet encapsulation rates; FIG. 15C depicts that vanillin secretion (VAN-3>VAN-2>VAN-1WT) is monitored over time, showing signal amplification during incubation; FIG. 15D depicts that the simultaneous vanillin and ATP secretion measurements reveal two clusters: VAN-3 and the negative control; FIG. 15E depicts that all yeast strains (VAN-1, VAN-2, VAN-3, and WT) show similar ATP secretion profiles; FIG. 15F depicts that the coupled assays identify three clusters (VAN-3, WT, and daughter cells) during vanillin secretion in mixed yeast samples (VAN-3: WT=1:1); and FIG. 15G depicts that the mixed WT and VAN-3 samples show distinct fluorescence peaks for sorting VAN-3 strains;

FIGS. 16A-16C depict the selective MOMS coating on mother yeast cells in droplets, in which: FIG. 16A depicts the budding of MOMS-coated yeast cells in droplets using bright-field and fluorescence microscopy; FIG. 16B depicts the flow cytometry confirming the high stability of MOMS coatings; and FIG. 16C depicts the flow cytometry analysis revealing that although the proportion of mother cells in the population decreases over time, the MOMS sensor density on mother yeast cells shows only a slight reduction;

FIGS. 17A-17B depict the metabolic pathway for engineering yeast to produce vanillin, in which: FIG. 17A shows a Schematic representation of the vanillin biosynthesis pathway from ferulic acid; and FIG. 17B depicts the gene circuit in plasmid pCYP-VAN, which harbors the 4cl and ech genes essential for vanillin synthesis;

FIG. 18 depicts the signal saturation analysis of MOMS, showing that the fluorescence intensity on the surface of the mother cells increases proportionally with the vanillin concentration in the droplets;

FIGS. 19A-19G depict the high-speed sorting for rapid directed evolution, in which: FIG. 19A depicts that MOMS efficiently screens a mixed population; FIG. 19B depicts that MOMS is used to screen a mutant library for ECH enzyme evolution; FIG. 19C depicts the time-course high-performance liquid chromatography (HPLC) analysis of fermentation supernatants from VAN-3 and selected mutant strains; FIG. 19D depicts a schematic illustrates the sorted ECH mutant from the S-10 strain, with amino acid substitutions: I90N, Y169C, N212Q, and P213L; FIG. 19E depicts that a library of 2×10⁷ yeast variants is screened using MOMS to isolate top-performing strains with enhanced extracellular vanillin secretion; FIG. 19F depicts that four high-performing transporter variants are validated through batch fermentation; and FIG. 19G depicts that two key mutations, K40R and V79D, are identified in the top-performing strain;

FIG. 20 depicts the validation of vanillin production via absorbance measurements;

FIG. 21 depicts the time-course measurement of vanillin concentrations in fermentation supernatant of the VAN-3 and mutant strains (S-1, S-8, and S-10);

FIGS. 22A-22B depict the additional mutation sites in ECH variants from sorted strains, in which: FIG. 22A shows a schematic of the sorted mutational ECH from S-1 strain, with the Y75H substitution; and FIG. 22B is a schematic of the sorted mutational ECH variant from S-8 strain, with the D80V substitution;

FIG. 23 depicts the validation of ECH enzyme activity through vanillin production in Escherichia coli;

FIGS. 24A-24C depict the quantitative analysis of ECH enzyme activity, in which: FIG. 24A depicts that ECH enzymes are purified from E. coli strains expressing either wild-type or mutant ech genes for enzymatic activity quantification;

FIG. 24B depicts that the equal concentrations of purified enzymes (1 mg/mL) are incubated with 300 μM feruloyl-CoA and 7.5 μM j-NAD at 30 °C for 12 hours; and FIG. 24C depicts that the enzyme activity is calculated based on vanillin yield;

FIG. 25 depicts the validation of vanillin secretion for sorted transporter mutants;

FIG. 26 depicts that the assessment of fluorescence signal variability associated with yeast cell size and budding state;

FIGS. 27A-27B depict the stringent gating strategy for minimizing fluorescence false positives caused by yeast cell size and budding variation, in which: FIG. 27A depicts that the top 0.05% of high-performing strains is isolated from the vanillin transporter mutant library; and FIG. 27B shows the comparison of FSC-A signal distributions between sorted and unsorted yeast populations;

FIGS. 28A-28C depict the additional mutation sites in PP_0179 vanillin transporter variants from sorted strains, in which: FIG. 28A is a schematic of the sorted mutational PP_0179 from the T-1 strain, with the G45C substitution; FIG. 28B depicts the schematic of the sorted mutational PP_0179 variant from the T-2 strain, with the K48M substitution; and FIG. 28C is a schematic of the sorted mutational PP_0179 variant from the T-3 strain, with the T15I/G45R/Q98H substitutions;

FIGS. 29A-29D depict the high-flexibility of MOMS for protein analysis, in which: FIG. 29A depicts that the dynamic detection range of IFN-γ is evaluated using aptamer-functionalized MOMS (aptamer-MOMS) over concentrations ranging from 1 to 1000 nM; FIG. 29B depicts that the specificity of glycosylated human serum albumin (gHSA)-MOMS is assessed by comparing responses to gHSA, bovine serum albumin (BSA), and non-glycosylated human serum albumin (HSA); FIG. 29C depicts that the antibody-functionalized MOMS (antibody-MOMS) are fabricated by coating biotinylated yeast cells with streptavidin-modified IFN-γ capture antibodies; and FIG. 29D depicts that the antibody-MOMS are incubated with varying concentrations of IFN-γ, and the captured proteins are labeled with APC-tagged detection antibodies to generate quantifiable fluorescence signals; and

FIG. 30 depicts the adaptation of the MOMS platform for use with Pichia pastoris cells.

### DETAILED DESCRIPTION

In the following description, systems and/or methods and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

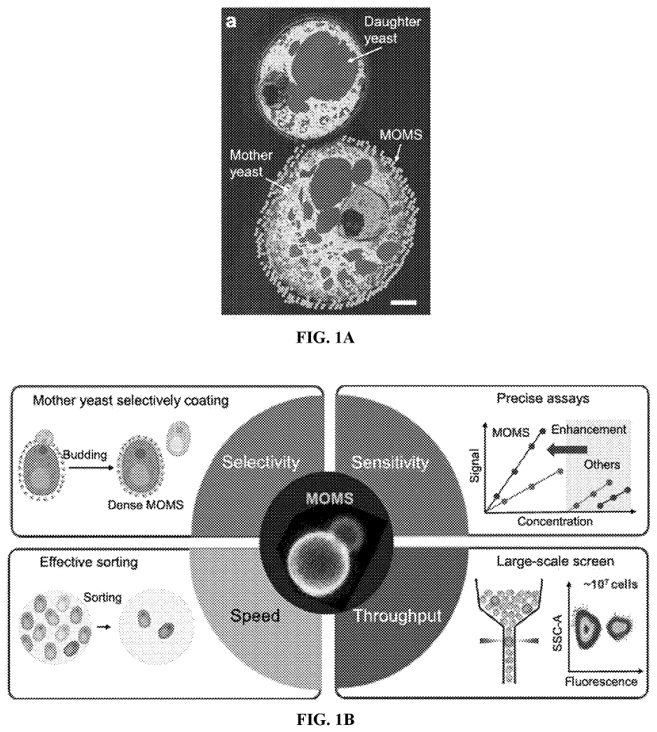

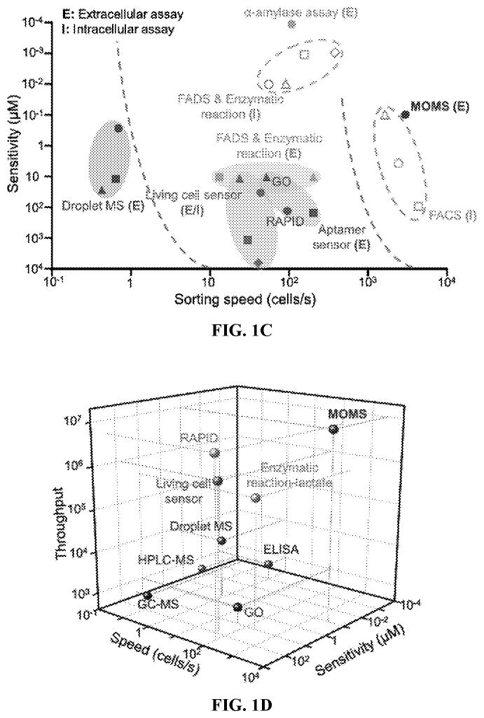

The present invention provides an advanced molecular sensing platform referred to as molecular sensors on the membrane surface of mother yeast cells (MOMS) for large-scale analysis of extracellular secretions from single yeast cells. The MOMS system utilizes aptamer-based molecular sensors that are selectively anchored to the membrane surface of mother yeast cells (FIG. 1A) and are not transferred to daughter cells during budding. This selective localization enables a high-density coating of molecular sensors on mother cells, reaching approximately 1.4×10⁷ sensors per cell, thereby enabling precise detection and quantification of secreted molecules from individual yeast cells.

The MOMS platform provides several key advantages. First, it offers high sensitivity, enabling detection of secreted metabolites at concentrations as low as approximately 100 nM (limit of detection, LOD). Second, the platform provides high throughput, as secretions are measured directly on the surface of yeast cells, allowing analysis of more than 10⁷ individual yeast cells with high efficiency. Third, the system provides high processing speed, enabling screening at rates of approximately 3.0×10³ cells per second and allowing isolation of rare high-secretion strains (approximately 0.05%) from large mutant libraries containing about 2.2×10⁶ variants within approximately 12 minutes (FIG. 1).

Compared with state-of-the-art droplet-based screening technologies, the MOMS platform demonstrates substantial performance improvements, including more than a ten-fold increase in sensitivity, greater than a two-fold increase in throughput, and more than a thirty-fold increase in processing speed for screening extracellular secretions from single yeast cells across a wide range of metabolic compounds (FIG. 1C). A comparative evaluation of MOMS with other analytical technologies used for microbial extracellular secretion analysis (FIG. 1D and Table 1) highlights the improved balance achieved by the MOMS platform in terms of sensitivity, screening throughput, and sorting speed.

To demonstrate the practical utility of the MOMS platform, the system is applied to directed evolution of yeast strains for improved metabolite production. Using this approach, yeast strains optimized for vanillin production are successfully identified, exhibiting secretion rates more than 2.7 times higher than those of their parental strains. Accordingly, the MOMS platform represents an advanced molecular sensing system for yeast metabolic analysis, providing an exceptional combination of sensitivity, speed, and throughput. The platform serves as a powerful tool for large-scale screening of extracellular secretions from single yeast cells and significantly expands the capabilities of metabolic analysis and biofabrication.

TABLE 1
Comparative analysis of main technologies
for single yeast cell secretion analysis

| | | | Screening | Sorting |
|---|---|---|---|---|
| | | | throughput | speed |
| Platform | Sensitivity | | (cells/assay) | (cells/s) |
| Enzyme linked | 1 | nM | ~10³-10⁴ | ~1 |
| immunosorbent assay | | | | |
| (ELISA) | | | | |
| Gas chromatography- | 20 | μM | ~10²-10³ | ~0.03 |
| mass spectrometry (GC- | | | | |
| MS) | | | | |
| High-performance | 1 | nM | ~10²-10³ | ~0.05 |
| liquid chromatography- | | | | |
| mass spectrometry | | | | |
| (HPLC-MS) | | | | |
| Droplet enzyme | 10 | nM | ~7.2 × 10⁵ | ~100 |
| screening | | | | |
| RNA-Aptamer-in- | 260 | μM | ~5.0 × 10⁶ | ~100 |
| Droplet (RAPID) | | | | |
| Living-cell biosensors in | 70 | μM | ~10⁶ | ~10 |
| droplet | | | | |
| graphene oxide (GO) | 10 | μM | ~10³-10⁴ | ~50 |
| aptasensors in droplet | | | | |
| Droplet mass | 250 | nM | ~10⁴ | 0.8 |
| spectrometry (MS) | | | | |
| MOMS (the present | 100 | nM | ~1.0 × 10⁷ | ~3000 |
| invention) | | | | |

In accordance with a first aspect of the present invention, a method for ultra-high-throughput screening of yeast metabolism is provided. The method includes providing a population of yeast cells and anchoring a plurality of molecular sensors on a membrane surface of mother yeast cells to form molecular sensors on membrane surfaces of mother cells. The molecular sensors are selectively retained on the mother yeast cells during budding and are not transferred to daughter cells, thereby maintaining a stable sensor population on the mother cell surfaces over successive cell divisions. The yeast cells are incubated under conditions that allow secretion of metabolic products from individual yeast cells. The secreted metabolic products are captured by the molecular sensors anchored on the membrane surface of the mother yeast cells. Upon interaction with the captured metabolic products, the molecular sensors generate detectable signals, which are subsequently used to screen the yeast cells and identify cells exhibiting a desired metabolic secretion phenotype. In certain embodiments, the secreted metabolic products comprise target metabolites and proteins.

In some embodiments, the secreted metabolic products include target metabolites and proteins, and the molecular sensors comprise nucleic acid aptamer sensors configured to bind to the target metabolites and proteins. The aptamer sensors may be immobilized on the membrane surface of the mother yeast cells via a biotin-streptavidin interaction, thereby enabling stable and high-density attachment of the molecular sensors on the cell surface. In certain embodiments, the molecular sensors are immobilized at a density of at least 10⁶ sensors per yeast cell, which enhances detection sensitivity by increasing the probability of capturing secreted molecules in proximity to the cell surface. In some implementations, the molecular sensors generate fluorescence signals upon binding to the secreted metabolic products, thereby enabling optical detection and quantitative measurement of secretion activity from individual yeast cells.

In certain embodiments, the method further comprises encapsulating individual MOMS-coated yeast cells in microfluidic droplets to capture secreted metabolites within the droplets. The droplets may provide isolated microenvironments that prevent cross-contamination between neighboring cells and facilitate accumulation of secreted products near the producing cell. Following incubation, the droplets may be disrupted to release the yeast cells for subsequent screening, such as through fluorescence-based detection or sorting methods.

In some embodiments, the molecular sensors comprise structure-switching aptamers that emit fluorescence upon binding to a target molecule. These aptamers undergo conformational changes when interacting with their targets, thereby generating a detectable fluorescence signal. In certain embodiments, the target metabolites include vanillin, ATP, glucose, and zinc ions. In other embodiments, the target proteins include interferon-gamma (IFN-7) and glycosylated human serum albumin. The method may be applied to yeast species including Saccharomyces cerevisiae or Pichia pastoris, although other budding yeasts may also be utilized.

In further embodiments, the method includes generating a mutant yeast library and performing directed evolution by selecting yeast variants exhibiting enhanced secretion phenotypes. Such variants may be identified through analysis of the detectable signals generated by the molecular sensors, allowing efficient enrichment of high-performing strains from large yeast populations. In some embodiments, multiplex detection of multiple metabolites is performed by anchoring different molecular sensors targeting different molecules on the membrane surface of the mother yeast cells. This multiplex configuration enables simultaneous monitoring of multiple secreted compounds from individual yeast cells, thereby expanding the analytical capability of the screening platform.

In accordance with a second aspect of the present invention, a functionalized yeast cell for detecting extracellular secretions is provided. The functionalized yeast cell include a mother yeast cell having a membrane surface and a plurality of molecular sensors immobilized on the membrane surface of the mother yeast cell. The molecular sensors are configured to bind secreted molecules produced by yeast cells and to generate detectable signals upon interaction with the secreted molecules. By immobilizing the molecular sensors on the membrane surface of the mother yeast cell, secreted molecules released from the yeast cell can be captured in close proximity to their source, thereby enabling sensitive detection of extracellular secretion events. Importantly, the molecular sensors remain localized on the mother yeast cell during budding and are substantially not transferred to daughter cells, allowing the mother yeast cell to retain a stable population of sensors over multiple rounds of cell division. This selective localization ensures that signal generation corresponds specifically to secretion events associated with the mother yeast cell and avoids dilution of sensor density during cell proliferation.

In certain embodiments, the molecular sensors include aptamer sensors capable of specifically recognizing target molecules. Such aptamer sensors may be designed to bind a variety of metabolites or proteins secreted by yeast cells, thereby enabling selective detection of diverse extracellular molecules. In some embodiments, the molecular sensors are immobilized on the membrane surface of the mother yeast cell through biotin-streptavidin binding, which provides a stable and high-affinity interaction for anchoring the sensors to the cell surface. The use of biotin-streptavidin interactions facilitates efficient attachment of the sensors while maintaining their functional conformation and molecular recognition capability.

In further embodiments, the molecular sensors include fluorescence-based structure-switching aptamers. These aptamers undergo conformational changes upon binding to target molecules, resulting in the generation of detectable fluorescence signals. The fluorescence-based detection mechanism allows real-time monitoring and quantitative analysis of extracellular secretion events at the single-cell level. By employing structure-switching aptamers as molecular sensors, the functionalized yeast cells serve as sensitive platforms for detecting extracellular metabolites and proteins while maintaining the physiological activity and viability of the yeast cells.

In accordance with a third aspect of the present invention, a system for ultra-high-throughput screening of microbial secretions is provided. The system includes at least one of the aforementioned functionalized yeast cell, wherein the functionalized yeast cell includes a mother yeast cell having a membrane surface and a plurality of molecular sensors immobilized on the membrane surface of the mother yeast cell. The molecular sensors are configured to bind secreted molecules produced by yeast cells and generate detectable signals upon interaction with the captured molecules. In the system, the molecular sensors remain localized on the mother yeast cell during budding and are substantially not transferred to daughter cells, thereby maintaining a stable sensing interface on the mother yeast cell for monitoring extracellular secretion events.

The system further includes a droplet microfluidic device configured to encapsulate individual yeast cells in droplets. The droplet microfluidic device is capable of generating discrete droplets that serve as isolated microenvironments for individual yeast cells, thereby enabling single-cell analysis of secretion behavior. By encapsulating individual yeast cells in droplets, the secreted molecules can accumulate within the confined droplet volume and be captured efficiently by the molecular sensors immobilized on the membrane surface of the mother yeast cells.

The system additionally includes an incubation module configured to allow secretion and capture of metabolites by the molecular sensors. During incubation, the encapsulated yeast cells are maintained under conditions suitable for cell growth and metabolic activity, thereby enabling secretion of metabolites or proteins from the yeast cells. The molecular sensors immobilized on the membrane surface of the mother yeast cells capture the secreted molecules and generate detectable signals corresponding to the secretion activity of individual cells.

The system further includes a detection and sorting device configured to detect signals generated by the molecular sensors and to isolate yeast cells exhibiting desired secretion phenotypes. The detection and sorting device may analyze the detectable signals produced by the molecular sensors and identify yeast cells that exhibit enhanced or otherwise desired secretion characteristics. Based on the detected signals, the system is capable of isolating or sorting yeast cells exhibiting the desired secretion phenotypes for further analysis, cultivation, or directed evolution.

The molecular sensors include aptamer sensors configured to bind secreted metabolites. These aptamer sensors provide selective molecular recognition of target metabolites, enabling the system to detect specific metabolic products produced by yeast cells. By utilizing aptamer sensors as the molecular sensing elements, the system enables sensitive and selective detection of extracellular metabolites while supporting ultra-high-throughput screening of microbial secretion phenotypes.

Accordingly, the system is able to achieve screening at least 10⁷ yeast cells and sorting yeast cells at a rate of at least 10³ cells per second.

In accordance with a fourth aspect of the present invention, a mutant enoyl-CoA hydratase/aldolase (ECH) enzyme including at least one amino acid substitution selected from I90N, Y169C, and N212Q relative to a wild-type ECH enzyme is provided.

### EXAMPLES

TABLE 2
The primers used in examples

| | |
|---|---|
| Name | Sequences (5′-3′) |

Primers for deletion of gre2 and adh6 genes in S. cerevisiae BY4742

| | |
|---|---|
| ADH6-sgRNA-F | GATCGACATTAAGATCGAAGCATGTGGGTTTTAGAGC |
| | TAG (SEQ ID NO: 01) |
| ADH6-sgRNA-R | CTAGCTCTAAAACCCACATGCTTCGATCTTAATGTC |
| | (SEQ ID NO: 02) |
| ADH6-donorU-F | GGACCTTGACGTGGAATTCC (SEQ ID NO: 03) |
| ADH6-donorU-R | GGCTTTTCTTGTTGTTGTGTTG (SEQ ID NO: 04) |
| ADH6-donorD-F | CAGACTAGGTTGTCAAGCTCTTG (SEQ ID NO: 05) |
| ADH6-donorD-R | CATGGAACATCCTCATCCGC (SEQ ID NO: 06) |
| GRE2-sgRNA-F | GATCAAAGGCCGAGAATTTAACGGGTTTTAGAGCTA |
| | G (SEQ ID NO: 07) |
| GRE2-sgRNA-R | CTAGCTCTAAAACCCGTTAAATTCTCGGCCTTT (SEQ |
| | ID NO: 08) |
| GRE2-donorU-F | TGTACGCTATAGTTTCCTTTCAA (SEQ ID NO: 09) |
| GRE2-donorU-R | TTTACGGGCGTGTGATACTG (SEQ ID NO: 10) |
| GRE2-donorD-F | TCGTCCTTCTTGAAGTCCCA (SEQ ID NO: 11) |
| GRE2-donorD-R | CGACACTGCCTCCCAAATTT (SEQ ID NO: 12) |

Primers for construction of pVAN plasmid

| | |
|---|---|
| PTPI1-F | AACTTTGTATAGAAAAGTTGAAGGATGAGCCAAGAA |
| | TAAG (SEQ ID NO: 13) |
| PTPI1-R | TACAAACTTGTTTTAGTTTATGTATGTGTTTTTTGTAG |
| | (SEQ ID NO: 14) |
| 4CL-F | TAAACTAAAACAAGTTTGTACAAAAAAGCAG (SEQ |
| | ID NO: 15) |
| 4CL-R | TTCTTCAGACTTATTTTGGAAGGTCCCC (SEQ ID NO: |
| | 16) |
| TGPM1-F | TCCAAAATAAGTCTGAAGAATGAATGATTTG (SEQ ID |
| | NO: 17) |
| TGPM1-R | CAGTTGGATCTATTGCTATAACATGTCATGTC (SEQ ID |
| | NO: 18) |
| PFBA1-F | TATAGCAATAGATCCAACTGGCACCGCTG (SEQ ID |
| | NO: 19) |
| PFBA1-R | ATTTGCTCATTTTGAATATGTATTACTTGGTTATGGTTA |
| | TATATGACAAAAGAAAAAG (SEQ ID NO: 20) |
| ECH-F | CATATTCAAAATGAGCAAATACGAAGGAC (SEQ ID |
| | NO: 21) |
| ECH-R | TTTGTACAAGAAAGCTGGGTTCACCTCTTGTAAGCCT |
| | G (SEQ ID NO: 22) |
| pYCP-F | ACCCAGCTTTCTTGTACAAAG (SEQ ID NO: 23) |
| pYCP-R | CAACTTTTCTATACAAAGTTGGTG (SEQ ID NO: 24) |
| 4CL-colony-F | TGAGAGTGAGATGCCTGAAGTT (SEQ ID NO: 25) |
| 4CL-colony-R | ATTGCTTTATCTCCTCCTCCGT (SEQ ID NO: 26) |
| ECH-colony-F | TTTTCCTTCTTCTTCGCCCAC (SEQ ID NO: 27) |
| ECH-colony-R | TAGTCCTCGTTCTGTTCCCAC (SEQ ID NO: 28) |

Primers for constructing mutation library of ECH enzyme

| | |
|---|---|
| epECH-F | GTTCTTCCTTGCGTTATTCTTCTGTTCTTC (SEQ ID |
| | NO: 29) |
| epECH-R | CACTTTGTACAAGAAAGCTGGGTTCAC (SEQ ID NO: |
| | 30) |
| pYCP-L-F | GTGAACCCAGCTTTCTTGTACAAAGTG (SEQ ID NO: |
| | 31) |
| pYCP-L-R | GAAGAACAGAAGAATAACGCAAGGAAGAAC (SEQ |
| | ID NO: 32) |

Primers for construction of pET21b-ECH plasmid

| | |
|---|---|
| ECH-F2 | TTAACTTTAAGAAGGAGATATACATATGAGCAAATAC |
| | GAAGGACGTTGGA (SEQ ID NO: 33) |
| ECH-R2 | GGATCTCAGTGGTGGTGGTGGTGGTGCCTCTTGTAA |
| | GCCTGCAATCCAGGTT (SEQ ID NO: 34) |
| pET21b-ECH-F | AACCTGGATTGCAGGCTTACAAGAGGCACCACCACC |
| | ACCACCACTGAGATCC (SEQ ID NO: 35) |
| pET21b-ECH-R | TCCAACGTCCTTCGTATTTGCTCATATGTATATCTCCT |
| | TCTTAAAGTTAA (SEQ ID NO: 36) |

Primers for construction of pVAN-PP_0179 plasmid

| | |
|---|---|
| pVAN-GJ-F | GCGGCCCTGCATTAATGAATCGGCCAA (SEQ ID NO: |
| | 37) |
| pVAN-GJ-R | AGCTTGCAAATTAAAGCCTTCGAGCGTC (SEQ ID NO: |
| | 38) |
| PTPI1-F2 | TTGGGACGCTCGAAGGCTTTAATTTGCAAGCTAAGG |
| | ATGAGCCAAGAATAAGGGAACA (SEQ ID NO: 39) |
| PTPI1-R2 | TTTTAGTTTATGTATGTGTTTTTTGTAGTTATAG (SEQ |
| | ID NO: 40) |
| PP 0179-F | AACTACAAAAAACACATACATAAACTAAAAATGGTT |
| | CAAAGATTGACTTTCAGAAGACG (SEQ ID NO: 41) |
| PP 0179-R | AATCATCAAATCATTCATTCTTCAGACTTACTTAGCA |
| | GCTTTACGACCAGCCTTGGCA (SEQ ID NO: 42) |
| TGPM1-F2 | GTCTGAAGAATGAATGATTTGATGA (SEQ ID NO: 43) |
| TGPM1-R2 | GCGTTGGCCGATTCATTAATGCAGGGCCGCTATTGCT |
| | ATAACATGTCATGTCACCATT (SEQ ID NO: 44) |

Primers for constructing mutation library of PP_0179 transporter

| | |
|---|---|
| epPP_0179-F | TAACTACAAAAAACACATACATAAACTAAAAATG |
| | (SEQ ID NO: 45) |
| epPP_0179-R | GAAATCATCAAATCATTCATTCTTCAGACTTA (SEQ ID |
| | NO: 46) |

### Example 1. Fabrication of MOMS Platform

The fabrication of molecular sensors on the membrane surface of mother yeast cells involved three primary steps: (1) cell surface biotinylation, (2) preparation of an aptamer-streptavidin complex, and (3) coating of the aptamer complex onto the biotinylated yeast cells.

In the first step, biotin molecules are covalently conjugated to proteins present on the yeast cell surface. Yeast cells (2.0×10⁶ cells) are suspended in 90 μL of Dulbecco's phosphate-buffered saline (DPBS) supplemented with 5 mM MgCl₂, followed by the addition of 10 μL of 10 mM sulfo-NHIS-LC-biotin dissolved in DMSO. The reaction mixture is incubated at room temperature (approximately 25 °C) with shaking at 150 rpm for 30 minutes to allow surface biotinylation. The resulting biotinylated yeast cells are subsequently washed three times with 150 μL of DPBS containing 5 mM MgCl₂ to remove excess reagent.

In the second step, the aptamer-streptavidin complex is prepared by mixing biotin-labeled aptamers with streptavidin at a defined molar ratio (aptamer:streptavidin=3:1). Aptamer solutions (0.5, 1.0, 1.5, or 3.0 μL of a 100 μM solution) are combined with 0.63, 1.25, 2.50, or 5.00 μL of a 20 M streptavidin solution in 99 μL of DPBS containing 5 mM MgCl₂. The mixture is incubated at room temperature for approximately 5-10 minutes to allow formation of the aptamer-streptavidin complex. To stabilize the complex and minimize nonspecific interactions, 1 μL of 10 mg/mL herring sperm DNA is subsequently added.

In the third step, the prepared aptamer-streptavidin complex is coated onto the surface of the biotinylated yeast cells. The biotinylated yeast cells are resuspended in 100 μL of the aptamer-streptavidin solution and incubated at room temperature with shaking at 150 rpm for 30 minutes. After incubation, the cells are washed three times with 150 μL of DPBS containing 5 mM MgCl₂. The resulting functionalized yeast cells are then ready for use in secretion assays.

To enable detection of metabolic compounds, double-stranded DNA aptamer sensors are incorporated into the MOMS system. Biotin- and fluorescent-labeled aptamers (for example, biotin-Xapt-Cy5, biotin-Xapt-Cy3, or biotin-Xapt-FITC, where X denotes the target molecule) are first hybridized with complementary DNA (cDNA) strands labeled with quenching probes (for example, BHQ3 for Cy5 quenching, BHQ2 for Cy3 quenching, and BHQ1 for FITC quenching) to form quenched DNA duplex structures. In a typical preparation, the biotin-labeled aptamer is incubated with approximately 2.5-4.0 equivalents of its quenching cDNA in 50 μL of DPBS containing 5 mM MgCl₂ at room temperature for more than 2 hours. The resulting duplex structure quenches the fluorescence of the aptamer until activation by the target molecule.

The prepared DNA duplex is then incubated with approximately three equivalents of streptavidin (relative to the aptamer concentration) in 100 μL of DPBS containing 5 mM MgCl₂ and 0.1 mg/mL herring sperm DNA to generate a DNA-streptavidin complex. This complex is subsequently coated onto the biotinylated yeast cells using the procedure described above. The resulting functionalized yeast cells serve as versatile sensing platforms for biochemical and fluorescence-based assays.

In the present invention, aptamer sensors targeting different molecules are prepared as follows. For vanillin sensors, 3 μL of 100 μM biotin-VANapt-Cy5 or biotin-VANapt-FTC is hybridized with 7.5 μL of 100 μM BHQ3-VANQ or BHQ1-VANQ. For ATP sensors, 3 μL of 100 μM biotin-ATPapt-Cy5 is hybridized with 7.5 μL of 100 μM BHQ3-ATPQ. For glucose sensors, 3 μL of 100 μM biotin-GLUapt-Cy5 or biotin-GLUapt-Cy3 is hybridized with 12 μL of 100 μM BHQ3-GLUQ or BHQ2-GLUQ. For Zn²⁺ sensors, 3 μL of 100 μM biotin-ZINapt-Cy5 is hybridized with 12 μL of 100 DM BHQ3-ZINQ. For IFN-γ sensors, 3 L of 100 biotin-IFNapt-Cy5 is hybridized with 7.5 μL of 100 μM BHQ3-IFNQ. For gHSA sensors, 3 μL of 100 μM biotin-HSAapt-Cy5 is hybridized with 7.5 GL of 100 BHQ3-HSAQ. The sequences of all aptamers used in the present invention are provided in Table 3.

TABLE 3
The aptamer sequences and details

| Aptamer | Description | Sequences (5′-3′) |
|---|---|---|
| Biotin-ATPapt-Cy5 | ATP aptamer labeled by | /Cy5/TACCTGGGGGAGTATTG |
| | Cy5 | CG GAGGAAGGT (SEQ ID |
| | | NO: 47)/SpacerC9/Biotin/ |
| BHQ3-ATPQ | Quenching cDNA to | CTCCCCCAGGTA (SEQ ID |
| | Biotin-ATPapt-Cy5 | NO: 48)/BHQ3/ |
| Biotin-GLUapt-Cy5 | D-glucose aptamer labeled | /Cy5/CTCTCGGGACGACCGTG |
| | by Cy5 | TGTGTTGCTCTGTAACAGTGT |
| | | CCATT GTCGTCCC (SEQ ID |
| | | NO: 49)/SpacerC9/Biotin/ |
| BHQ3-GLUQ | Quenching cDNA to | GTCGTCCCGAGAG (SEQ ID |
| | Biotin-GLUapt-Cy5 | NO: 50)/BHQ3/ |
| Biotin-GLUapt-Cy3 | D-glucose aptamer labeled | /Cy3/CTCTCGGGACGACCGTG |
| | by Cy3 | TGTGTTGCTCTGTAACAGTGT |
| | | CCATTGTCGTCCC (SEQ ID |
| | | NO: 49)/SpacerC9/Biotin/ |
| BHQ2-GLUQ | Quenching cDNA to | GTCGTCCCGAGAG (SEQ ID |
| | Biotin-GLUapt-Cy3 | NO: 50)/BHQ2/ |
| Biotin-VANapt-Cy5 | Vanillin aptamer labeled | /Biotin/SpacerC9/CAGGAGAAA |
| | by Cy5 | CATGGAGTCTCGATGAT (SEQ |
| | | ID NO: 51)/Cy5/ |
| BHQ3-VANQ | Quenching cDNA to | /BHQ3/TCATCGAGAC (SEQ ID |
| | Biotin-VANapt-Cy5 | NO: 52) |
| Biotin-VANapt- | Vanillin aptamer labeled | /Biotin/SpacerC9/CAGGAGAAA |
| FITC | by FITC | CATGGAGTCTCGATGAT (SEQ |
| | | ID NO: 51)/FITC/ |
| BHQ1-VANQ | Quenching cDNA to | /BHQ1/TCATCGAGAC (SEQ ID |
| | Biotin-VANapt-FITC | NO: 52) |
| Biotin-ZINapt-Cy5 | Zinc ion aptamer labeled | /Cy5/CCATCAGTTAGTCATTAC |
| | by Cy5 | GCTTACGGCGGCTCTATCCTA |
| | | ACTGATATATTGTGAAGTCGT |
| | | GTCCC (SEQ ID NO: 53)/ |
| | | SpacerC9/Biotin/ |
| BHQ3-ZINQ | Quenching cDNA to | ATGACTAACTGATGG (SEQ ID |
| | Biotin-ZINapt-Cy5 | NO: 54)/BHQ3/ |
| Biotin-IFNapt-Cy5 | IFN-γ aptamer labeled by | /Cy5/TGGGGTTGGTTGTGTTG |
| | Cy5 | GG TGTTGTGT (SEQ ID |
| | | NO: 55)/SpacerC9/Biotin/ |
| BHQ3-IFNQ | Quenching cDNA to | ACAACCAACCCCA (SEQ ID |
| | Biotin-IFNapt-Cy5 | NO: 56)/BHQ3/ |
| Biotin-HSAapt-Cy5 | gHSA aptamer labeled by | /Biotin/SpacerC9/GGTGCGGTTC |
| | Cy5 | GTGCGGTTGTAGTACTCGTGG |
| | | CCG (SEQ ID NO: 57)/Cy5/ |
| BHQ3-HSAQ | Quenching cDNA to | /BHQ3/CGGCCACGA |
| | Biotin-HSAapt-Cy5 | |
| Biotin-NC-Cy5 | Negative control DNA | /Biotin/SpacerC9/CTCATTCAAT |
| | labeled by Cy5 | ACCCTACGTCTACCCTAC |
| | | (SEQ ID NO: 58)/Cy5/ |
| BHQ3-NCQ | Quenching cDNA to | /BHQ3/GTAGGGTAGACG (SEQ |
| | Biotin-NC-Cy5 | ID NO: 59) |
| Biotin-NC-FITC | Negative control DNA | /Biotin/SpacerC9/CTCATTCAAT |
| | labeled by FITC | ACCCTACGTCTACCCTAC |
| | | (SEQ ID NO: 58)/FITC/ |
| BHQ1-NCQ | Quenching cDNA to | /BHQ1/GTAGGGTAGACG (SEQ |
| | Biotin-NC-FITC | ID NO: 59) |

In one embodiment, yeast cells are first treated with sulfo-NHS-LC-biotin to biotinylate proteins present on the yeast cell wall. This is followed by sequential attachment of streptavidin and biotin-labeled DNA aptamers. The charged sulfonyl group of the biotinylation reagent prevents penetration through the cell membrane, thereby ensuring that biotinylation occurs exclusively on the cell surface and providing stable grafting sites for subsequent aptamer attachment. The aptamer sequences were specifically designed to recognize a range of target molecules, including ATP, glucose, vanillin, and zinc ions (Zn²⁺), thereby enabling the MOMS platform to capture diverse secreted metabolites and proteins.

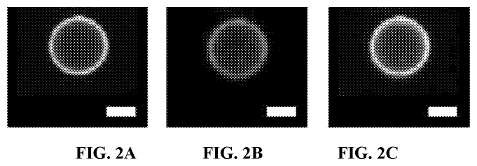

The presence of the MOMS coating is visualized by anchoring Cy5-labeled aptamers (excitation: 646 nm; emission: 664 nm) onto the yeast cell surface. In parallel, the yeast cell walls are stained with Alexa Fluor 488-Concanavalin A (ConA) (excitation: 495 nm; emission: 520 nm). Confocal laser scanning microscopy (CLSM) imaging confirms that the aptamer sensors are localized exclusively on the cell surface without detectable internalization. Importantly, the coating process did not affect yeast cell physiology or secretion profiles (FIGS. 2A-2C), indicating that the intrinsic cellular functions are preserved.

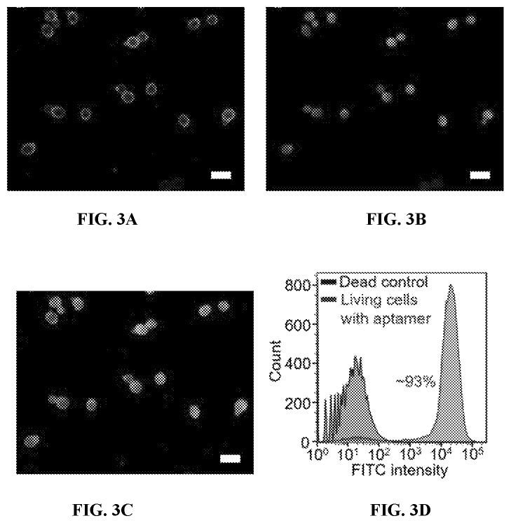

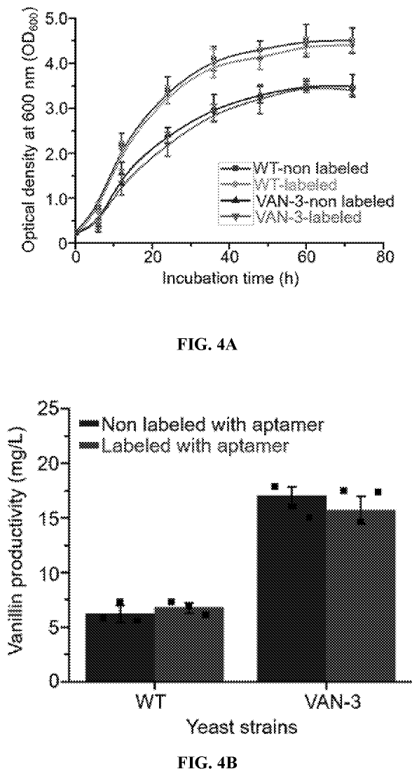

Following the MOMS coating procedure, the viability of yeast cells is further evaluated using fluorescein diacetate (FDA), which generates a fluorescent signal upon hydrolysis by intracellular esterases in living cells. CLSM imaging and flow cytometry analysis reveal a high fraction of viable cells (>93%) (FIGS. 3A-3D). Moreover, yeast cells functionalized with the MOMS coating retain normal proliferation and secretion behavior comparable to native, unmodified cells. These results confirm that the MOMS modification does not adversely affect cellular health or functionality (FIGS. 4A-4D).

To assess the biocompatibility of MOMS, yeast viability and growth are evaluated. For the viability test, yeast cells coated with fluorescently labeled single-stranded DNA (biotin-VANapt-Cy5) are suspended in 199 μL of DPBS and supplemented with 1 μL of 5 mg/m LFDA (dissolved in acetone). The suspension is incubated at 30 °C while shaking at 150 rpm for 15 minutes. After incubation, yeast cells are analyzed using CLSM imaging and flow cytometry. For the growth test, yeast cells with or without MOMS coatings are suspended in 5 mL of SD medium with an initial optical density at 600 nm (OD₆₀₀) of approximately 0.2. Cultures are incubated at 30 °C while shaking at 250 rpm for 72 hours, and cell density (OD₆₀₀) is measured at 12-hour intervals using a microplate reader.

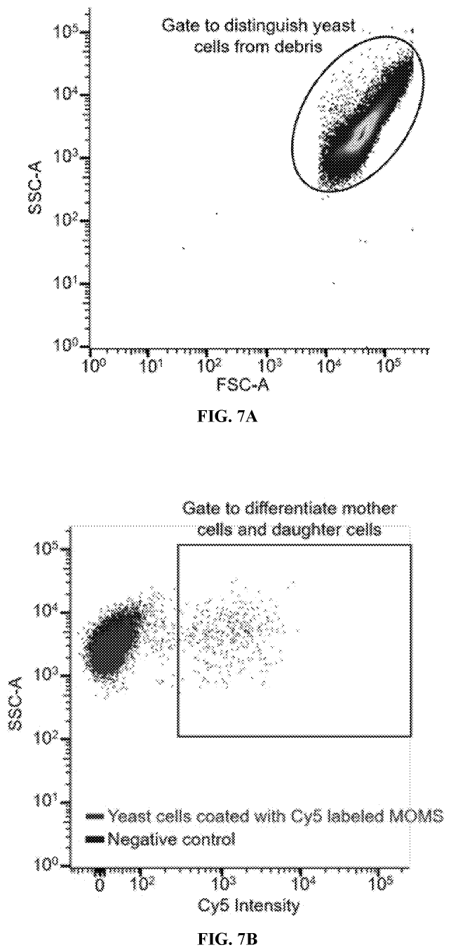

Briefly, the flow cytometry methodology is conducted with the following descriptions. An optimized gating strategy is applied to all flow cytometry experiments. To distinguish the target yeast population from debris, an initial gate is defined based on forward scatter area (FSC-A) and side scatter area (SSC-A) signals (FIG. 7A). To further differentiate mother yeast cells coated with fluorophore-labeled MOMS from daughter cells, a secondary gate is established using fluorescence-negative control samples (FIG. 7B). For sorting secretory strains, a stringent gating strategy is employed to minimize false-positive signals arising from variations in cell size or budding. Specifically, only the top 0.05% of mutant yeast cells exhibiting the highest fluorescence intensities are sorted.

### Example 2. Selective Dense MOMS Coating on Mother Yeasts

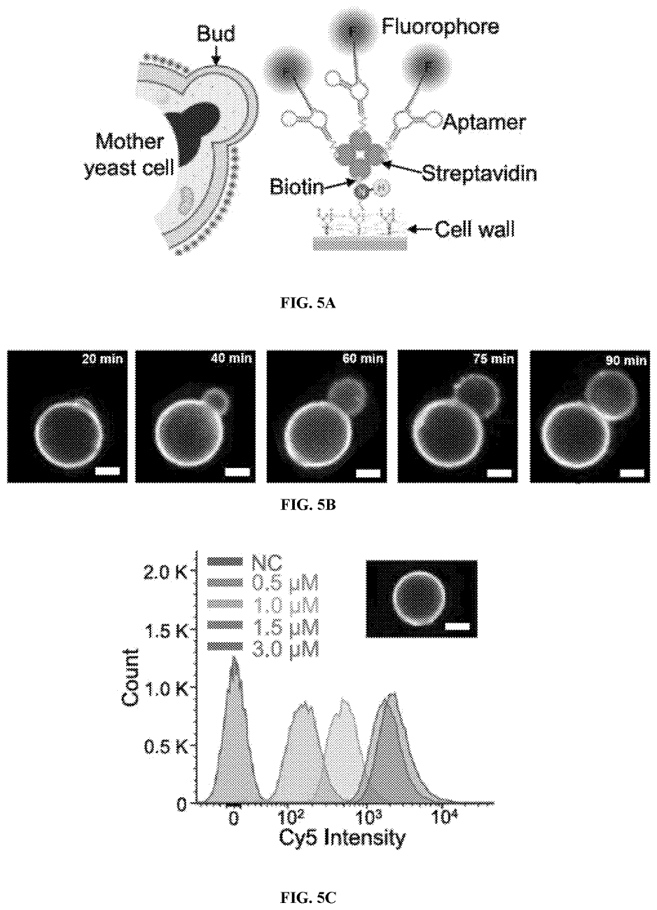

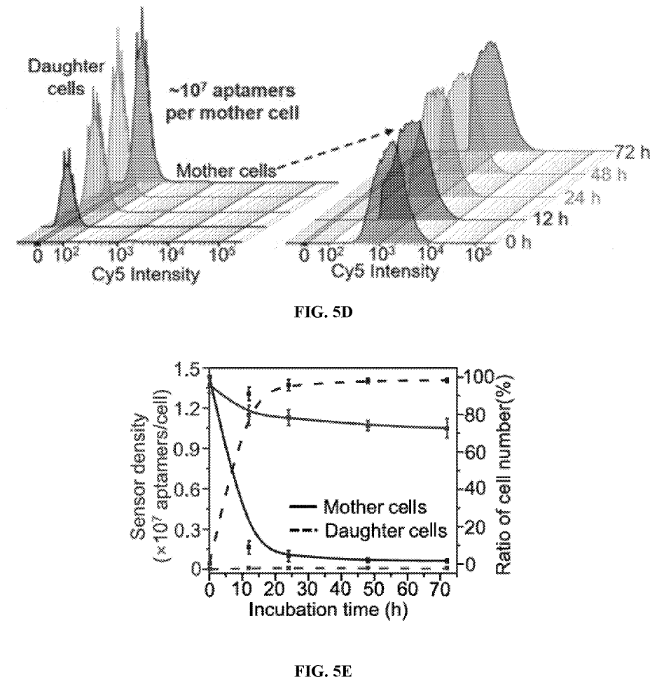

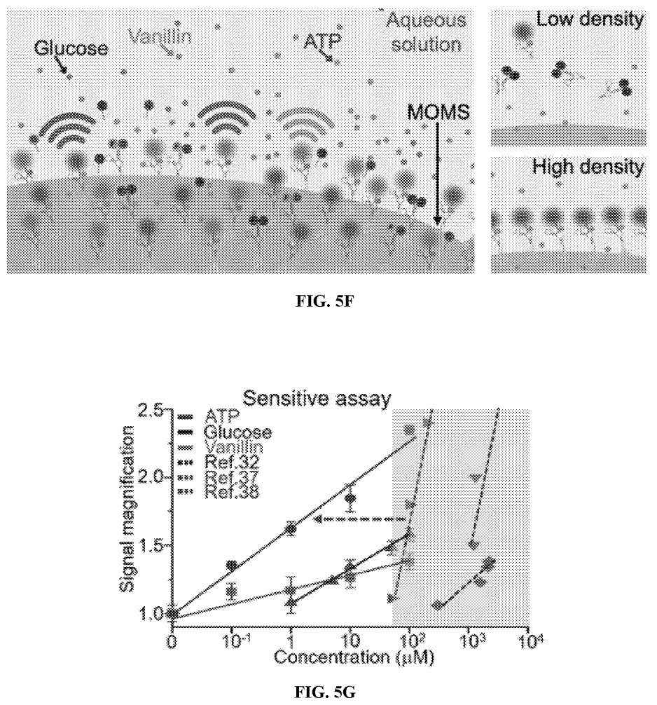

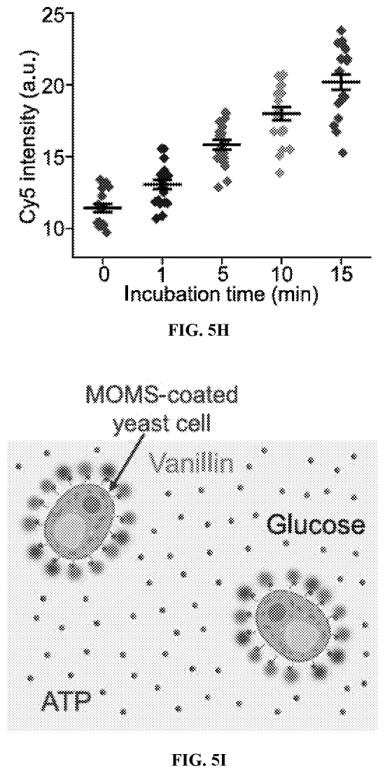

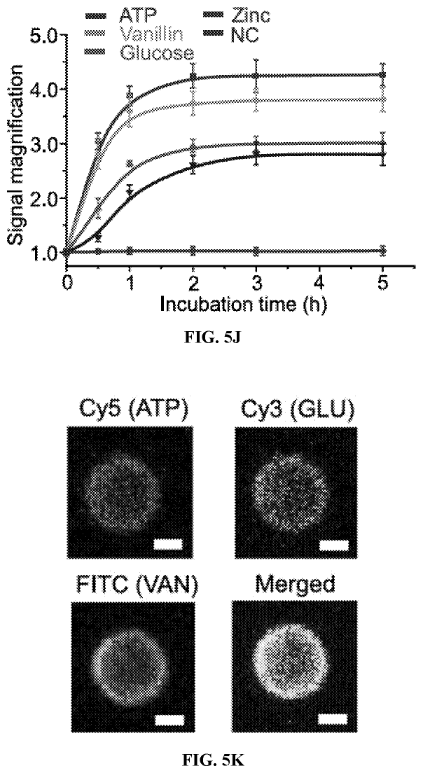

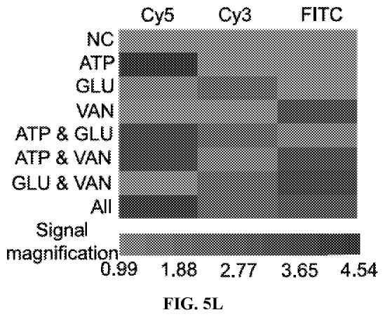

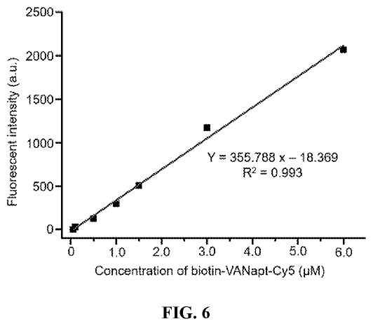

MOMS are coated onto yeast cell surfaces through functional biotin groups that bind to biotin-labeled aptamers via a streptavidin-mediated sandwich structure (FIG. 5A). Selective coating of MOMS on mother yeast cells is validated by anchoring Cy5-labeled aptamers to the cell surface, followed by staining of the yeast cell wall with Alexa Fluor 488-labeled Concanavalin A (ConA). This dual labeling confirms that the aptamer sensors are exclusively anchored to mother cells during proliferation. As daughter cells bud and form newly synthesized membranes, the MOMS coating remains confined to the original mother cells. This selective localization maintains a high density of molecular sensors on mother cells and generates distinct fluorescence signals that clearly differentiated mother cells from daughter cells (FIG. 5B).

To quantify the density of MOMS on the yeast surface, yeast cells at a fixed concentration (2.0×10⁷ cells/mL) are incubated with varying concentrations of aptamers in a 100 L medium. Sensor density is then analyzed by flow cytometry (FIG. 5C) and calibrated using a fluorescence plate reader (FIG. 6). Increasing the aptamer concentration from 0.5 μM to 1.5 M results in a proportional increase in fluorescence intensity, indicating a corresponding increase in sensor density on the cell surface. A saturation point is reached at approximately 1.4×10⁷ molecules per cell, beyond which fluorescence intensity remains constant, suggesting full occupancy of available binding sites on the yeast cell surface.

The stability of the MOMS coating on mother cells is further examined using flow cytometry (FIG. 5D). Mother cells coated with fluorophore-labeled MOMS are distinguished from daughter cells through appropriate gating strategies (FIG. 7). The results show that MOMS selectively coats mother yeast cells and are not transferred to daughter cells during budding. This selective localization remains stable over three days and more than 30 generations of cell division. Many mother cells retain dense MOMS coatings, as indicated by strong Cy5 fluorescence signals, while daughter cells remain largely uncoated.

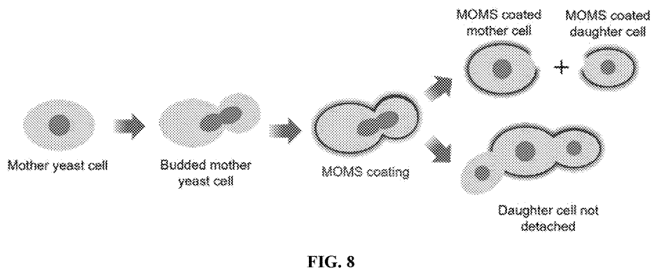

After prolonged budding (approximately 12 hours), a small fraction of MOMS (~16.4%) is observed to detach, leading to a slight reduction in fluorescence intensity. One possible explanation is that portions of some mother cells have already initiated the budding process prior to MOMS coating; as the coated buds continue to grow, they eventually detach from the mother cell surface during cytokinesis (FIG. 8). Despite this partial loss, the majority of the MOMS (~83.6%) remain on mother cells, demonstrating high stability suitable for long-term secretion measurements. Even after 72 hours of culture, a high sensor density (~1.0×10⁷ molecules per cell) is maintained on mother cells, although the proportion of mother cells in the population decreases due to extensive proliferation (FIG. 2E).

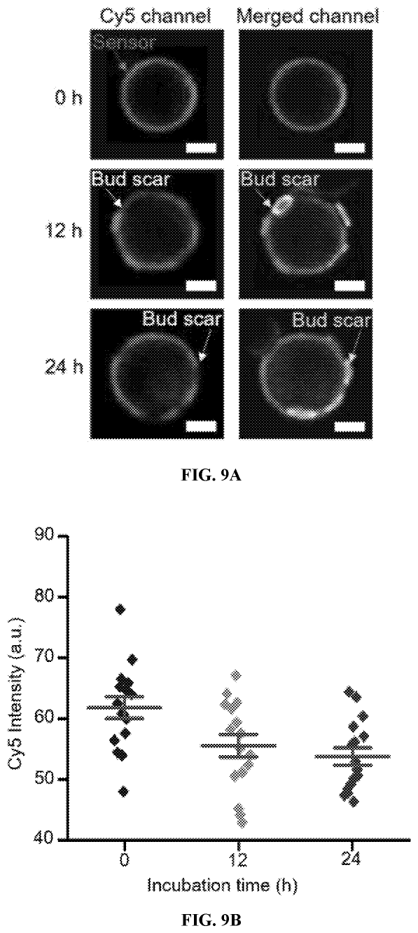

Confocal laser scanning microscopy (CLSM) images of mother yeast cells coated with Cy5-labeled aptamers further confirm that multiple generations of budding do not significantly affect the overall MOMS density on yeast cell surfaces (FIG. 9). Although the fraction of mother cells decreases over time, the density of MOMS on the remaining mother cells exhibits only a slight reduction, ensuring reliable long-term capture of secreted molecules. Importantly, the MOMS coating does not adversely affect yeast metabolic activity or proliferation, preserving the natural physiology of the cells and enabling accurate secretion profiling.

### Example 3. Sensitive Molecular Measurements Via MOMS

By grafting dense MOMS onto yeast cell surfaces, a highly sensitive assay capable of detecting target compounds through fluorescence signals generated by structure-switching aptamer sensors is developed. In this MOMS system, the fluorescent probe-labeled aptamer is initially quenched by a complementary DNA (cDNA) strand labeled with a quenching probe. Upon binding to a target molecule, the aptamer undergoes a conformational change that dissociates it from the cDNA, thereby restoring fluorescence. The high density of surface-immobilized sensors in the MOMS platform enables detection of low-abundance metabolic secretions, overcoming sensitivity limitations associated with existing sensing technologies (FIG. 5F).

To calibrate the sensing sensitivity of MOMS, yeast cells coated with MOMS are prepared by mixing 2.0×10⁶ biotinylated yeast cells with 3 M MOMS targeting specific molecules in 100 L of DNA-streptavidin solution. The mixture is incubated at 30 °C for 30 minutes. The coated yeast cells are then incubated in solutions containing known concentrations of target molecules (vanillin, ATP, glucose, and Zn²+) to determine their limits of detection (LODs). To minimize nonspecific binding of quenching cDNA detached from the aptamer to the yeast cell surface, 0.1 mg/mL Herring Sperm DNA is included in the buffer solution as a blocking agent. Tailored buffer solutions are used to optimize sensing conditions for different target molecules: Vanillin and ATP sensing: 10 mM Tris, 150 mM NaCl, and 5 mM MgCl₂; Glucose and Zn²⁺ sensing: DPBS supplemented with 5 mM MgCl₂. Following incubation at 30 °C with shaking at 150 rpm for a specified duration, yeast cells are washed and analyzed via flow cytometry. For specificity tests, MOMS-coated yeast cells targeting vanillin are incubated with 1 mM solutions of vanillin, vanillyl alcohol, vanillic acid, or ferulic acid to evaluate MOMS' capability for selective detection of the intended targets.

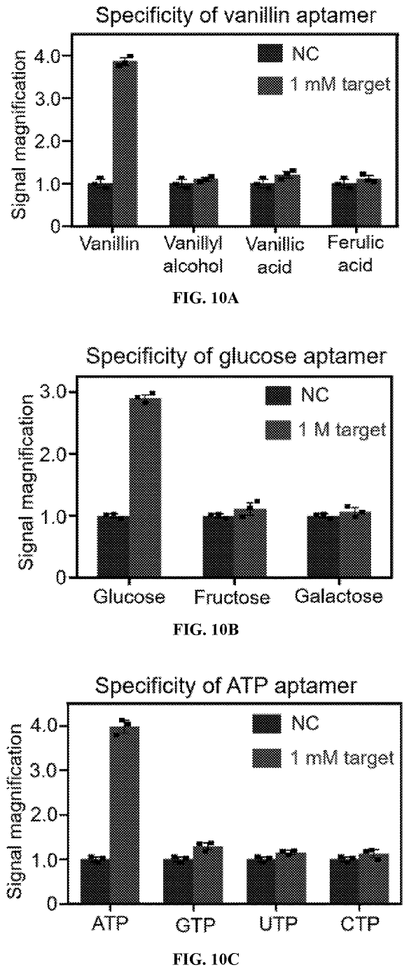

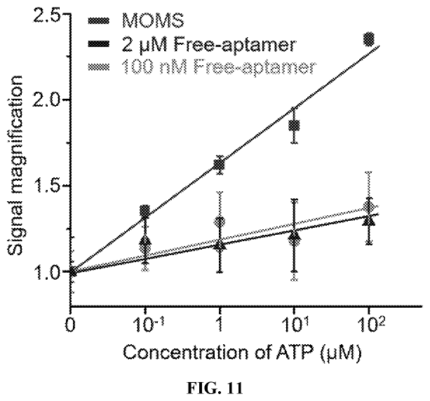

To validate assay specificity, vanillin-targeting aptamers are tested against structurally related compounds including vanillyl alcohol, vanillic acid, and ferulic acid. The MOMS platform generates a 3.86-fold fluorescence increase in response to vanillin, whereas the other compounds produce only minimal signal changes (1.06-1.18-fold), resulting in a specificity ratio of 3.45-fold (FIG. 10A). Similarly, in glucose specificity tests, exposure of MOMS to 1 M glucose results in a strong fluorescence increase (~3-fold), while fructose and galactose produce only minor responses, confirming the high molecular recognition specificity of the aptamer sensors (FIG. 10B). For ATP detection, ATP-specific MOMS produce a robust fluorescence increase (~4-fold) upon exposure to 1 mM ATP, whereas structurally related nucleotides-including GTP, UTP, and CTP-generate negligible fluorescence responses (FIG. 0C), further demonstrating the platform's excellent selectivity.

The dense MOMS coating enables detection of vanillin at concentrations as low as 100 nM, demonstrating a low detection limit. Similarly, detection limits for ATP and glucose are approximately 100 nM and 1 μM, respectively (FIG. 5G). Compared with conventional droplet assays using freely suspended aptamer sensors, the MOMS system substantially increases local sensor density, reaching approximately 1.4×10⁷ sensors per yeast cell. This increased sensor density results in more than a ten-fold improvement in sensitivity compared with droplet-based living-cell sensors and more than a hundred-fold improvement relative to freely suspended aptamer sensors within droplets.

To further confirm the sensitivity improvement provided by surface-immobilized MOMS, ATP aptamer sensors with identical structures are evaluated in a freely suspended state. These sensors (100 nM or 2 μM) are co-incubated with uncoated yeast cells (2.0×10⁷ cells/mL), a blocking agent (0.1 mg/mL herring sperm DNA), and varying concentrations of ATP. Fluorescence signals are measured using a microplate reader (FIG. 11). In contrast to the clear fluorescence increases observed with MOMS, freely suspended aptamers show only fluctuating signal differences across ATP concentrations ranging from 100 nM to 10 μM. This reduced sensitivity is likely caused by nonspecific interactions between the aptamers and yeast cell surfaces, which generate elevated background signals. By contrast, immobilization of aptamer sensors in the MOMS configuration minimizes nonspecific interactions, stabilizes sensor performance, and significantly improves detection sensitivity.

Although cell-free droplet systems containing enriched cellular extracts—such as tRNAs, nucleotides, and cofactors—can reduce background noise and improve detection sensitivity by eliminating membrane structures, their encapsulation efficiency for individual mutant DNA templates remains relatively low (~5-20%). Furthermore, many cell-free platforms are highly miniaturized and remain difficult to scale for industrial biomanufacturing. Even when promising biosynthetic pathways are identified in vitro, they must ultimately be reintroduced into living cells for functional validation.

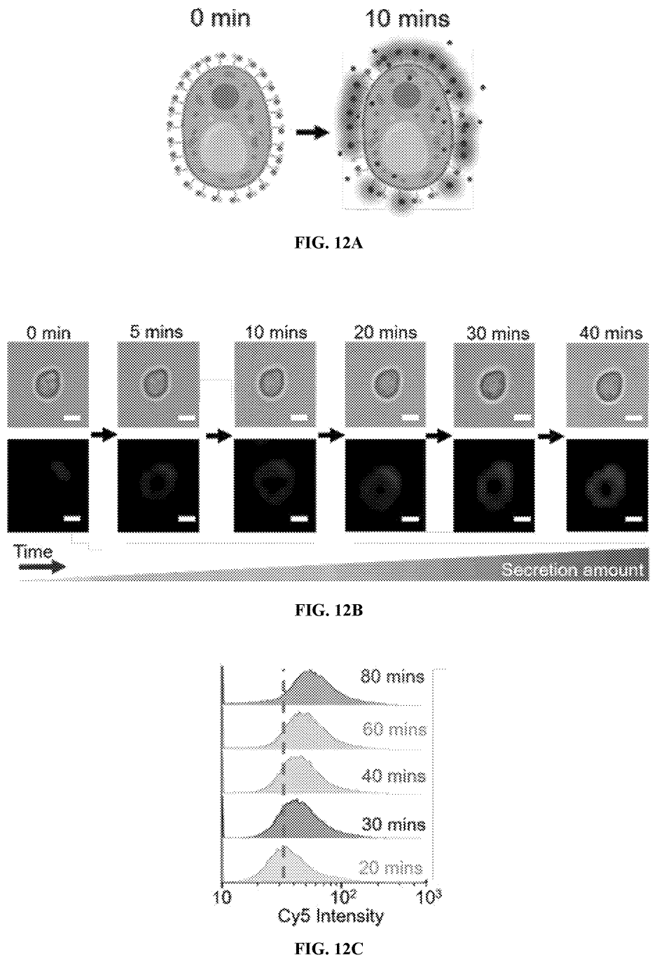

The high sensitivity of the MOMS platform enables visualization of secretions from individual Saccharomyces cerevisiae cells, as secreted compounds are directly captured by aptamer sensors anchored on the yeast cell surface (FIG. 12A). ATP secretion dynamics are monitored using confocal laser scanning microscopy (CLSM), with imaging performed at 5-minute intervals over a 40-minute period (FIG. 12B). Upon binding secreted ATP, the MOMS aptamers undergo conformational changes that activate Cy5 fluorescence (excitation: 646 nm; emission: 664 nm). Yeast cells dispersed in diluted culture medium (~5.0×10⁵ cells/mL) minimize cross-contamination via diffusion, allowing the MOMS coating on each cell to immediately capture ATP molecules upon secretion. Under normal culture conditions, even low-abundance ATP secretion from single yeast cells becomes detectable within 15 minutes and can be distinguished from baseline signals recorded at 0, 1, 5, and 10 minutes (FIG. 5H). Flow cytometry analysis further confirms the time-dependent increase in ATP secretion across individual yeast cells (FIG. 12C).

The high sensitivity and temporal resolution of the MOMS platform therefore provide unprecedented insights into secretion dynamics, enabling direct visualization and quantification of metabolic activity in individual yeast cells with exceptional precision.

Briefly, to validate the MOMS coating, yeast cells are labeled with fluorescently tagged single-stranded DNA. Specifically, 3 μM biotin-VANapt-Cy5 (excitation: 646 nm, emission: 664 nm) is incubated with a suspension of 1.0×10⁶ biotinylated yeast cells (wild-type S. cerevisiae strain BY4742) for fluorescent labeling. After incubation, the labeled yeast cells are treated with 2.5 M Alexa Fluor 488-ConA (excitation: 495 nm, emission: 520 nm) in 100 μL DPBS at 37 °C with shaking at 150 rpm for 30 minutes. The yeast cells are subsequently washed once and resuspended in either DPBS or YPD medium for imaging using a confocal laser scanning microscope.

To systematically quantify the density of the MOMS coating, 2.0×10⁶ yeast cells are incubated with varying concentrations of biotin VANapt-Cy5 (0.5-3.0 M) and analyzed using a flow cytometer. Yeast cells without fluorescently labeled DNA coating serve as negative controls. For each sample, 3.0×10⁵ yeast cells are analyzed. To estimate the number of sensor molecules on each yeast cell, fluorescence signals from solutions with known biotin-VANapt-Cy5 concentrations are calibrated using a microplate reader to generate a standard curve. The MOMS density per yeast cell can be calculated by determining the remaining biotin-VANapt-Cy5 concentration in the incubation solution post-coating (FIG. 6).

### Example 4. Sensitive Multiplexed Assay Via Combinational MOMS

By coating yeast cells with different MOMS, each incorporating distinct aptamer sequences, multiple target metabolites can be detected simultaneously, enabling multiplexed assays (FIG. 5I).

For multiplexed sensing, three types of MOMS with distinct aptamer sequences are simultaneously coated onto the same yeast cells. The MOMS specifically target ATP, vanillin, and glucose and are fabricated according to established protocols, with each labeled using distinct fluorescent dyes: Cy5 for ATP (excitation: 646 nm, emission: 664 nm), FITC for vanillin (excitation: 490 nm, emission: 525 nm), and Cy3 for glucose (excitation: 555 nm, emission: 570 nm). These MOMS (1.5 M each) are co-incubated with 2.0×10⁶ biotinylated yeast cells in a DNA-streptavidin solution containing 1.5 μM streptavidin and 0.1 mg/mL Herring Sperm DNA, resulting in a total reaction volume of 200 μL. After multiplex MOMS coating, the yeast cells are incubated in solutions containing various target molecules (0.5 mM ATP, 1.0 mM vanillin, and/or 1.0M glucose) in a buffer composed of 10 mM Tris, 150 mM NaCl, 5 mM MgCl₂, and 0.1 mg/mL Herring Sperm DNA. The incubation took place at 30 °C with shaking at 150 rpm for 1 hour. Following the incubation, the MOMS-coated yeast cells are washed and analyzed using flow cytometry to assess their sensing capacity for multiplexed assays.

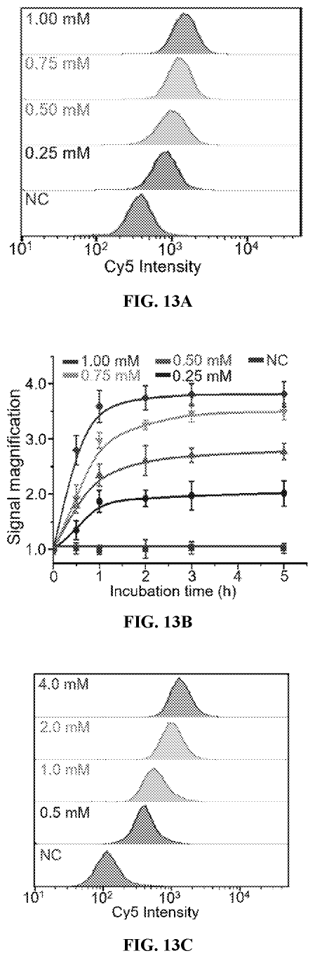

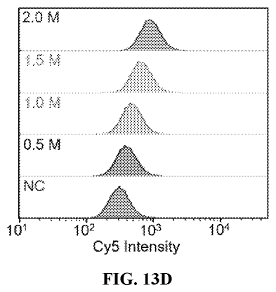

To evaluate the sensing performance of the system, MOMS-coated yeast cells are incubated with vanillin, ATP, glucose, or Zn²⁺ under various conditions over time (FIG. 5J; FIGS. 13A-13D). In the vanillin assay, fluorescence signals increase significantly within 1 hour of incubation and reach saturation after approximately 2 hours. At a vanillin concentration of 1 mM, a 3.82-fold signal amplification is observed, with a dynamic detection range of 100 nM-1 mM. For ATP detection, fluorescence signals exhibit a 9.81-fold amplification at 4 mM ATP, with saturation occurring within approximately 2 hours and a dynamic detection range of 100 nM-4 mM. In the glucose assay, fluorescence signals gradually plateau after about 2 hours, producing a 3.00-fold signal amplification at 1.5 M glucose and a dynamic detection range of 0-2 M. For Zn²⁺ detection, fluorescence signals reach a plateau after approximately 3 hours, with a 2.78-fold signal amplification observed at 2 mM Zn²⁺. These results demonstrate the versatility of the MOMS platform for high-sensitivity detection of a wide range of small molecules.

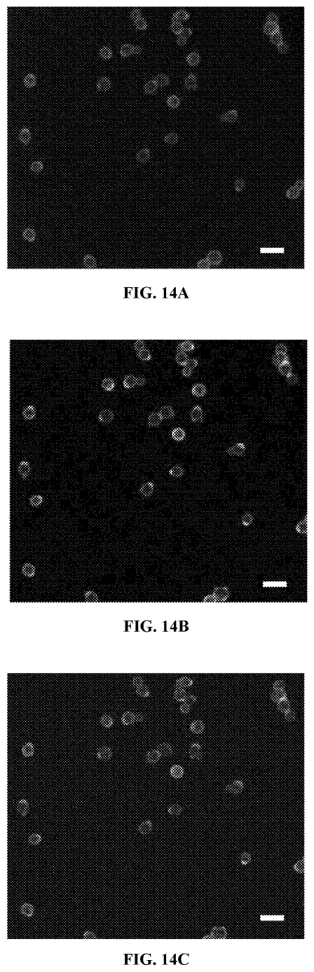

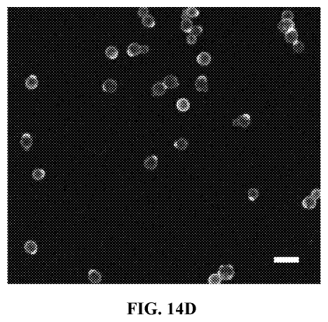

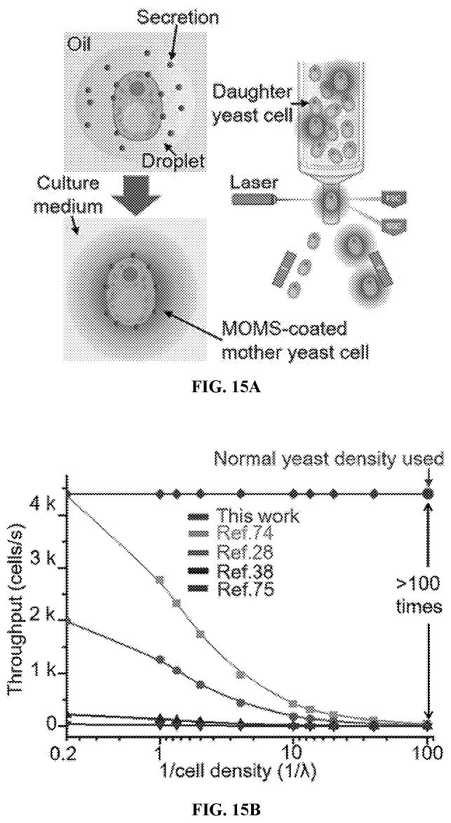

To further demonstrate multiplexing capability, three different types of MOMS are simultaneously grafted onto the surfaces of the same yeast cells, each functionalized with aptamers targeting vanillin, ATP, or glucose (FIG. 5K; FIGS. 14A-14D). This combinatorial MOMS system produces distinct fluorescence signals for each target molecule: ATP is detected using Cy5 fluorescence (excitation: 646 nm; emission: 664 nm), vanillin using FITC fluorescence (excitation: 490 nm; emission: 525 nm), and glucose using Cy3 fluorescence (excitation: 550 nm; emission: 570 nm). When exposed to a mixed solution containing all three target molecules, MOMS-coated yeast cells emit clearly distinguishable fluorescence signals corresponding to each metabolite. Flow cytometry analysis further confirms the ability of the system to differentiate yeast populations based on their fluorescence profiles, effectively isolating cells exhibiting multiple target signals from negative control populations with low fluorescence levels. Heatmap analysis of various mixtures of target compounds further confirms the accuracy of the MOMS assay, demonstrating 100% specificity in multiplexed measurements (FIG. 5L).

### Example 5. High-Throughput Screening Using MOMS

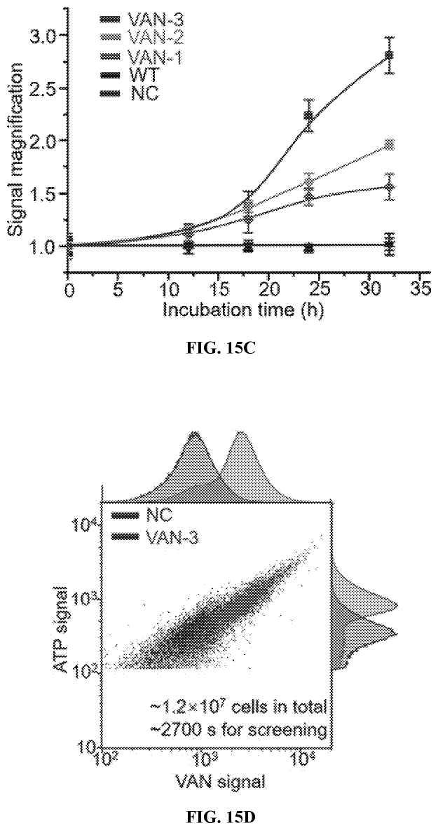

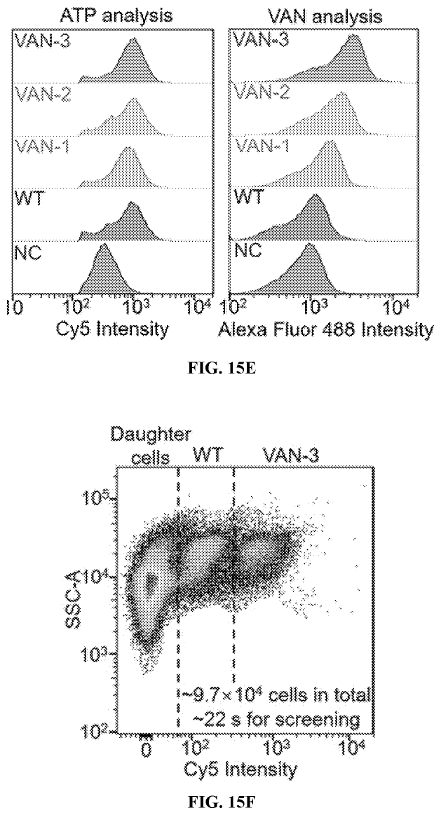

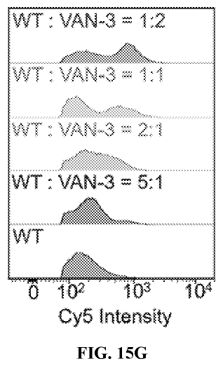

The high-throughput screening workflow includes two main steps: capturing and screening (FIG. 15A).

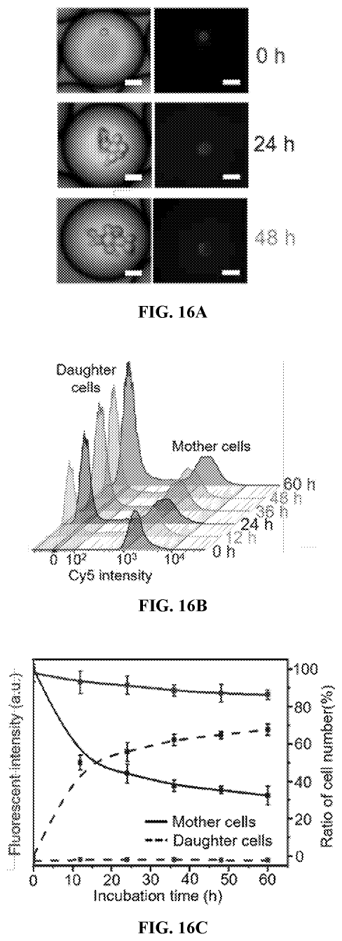

Step 1: Capturing. Droplet microfluidics are used to encapsulate individual MOMS-grafted yeast cells. Yeast cells coated with MOMS are suspended in YPD medium containing ferulic acid as the precursor substrate for vanillin production and encapsulated into approximately 33-μL water-in-oil droplets stabilized by a biocompatible surfactant. A cell concentration of approximately 1.5×10⁶ cells/mL ensures a single-cell encapsulation probability of less than 5%, with a co-encapsulation probability of less than 0.13% according to the Poisson distribution. The encapsulated yeast cells are incubated in droplets for approximately 48 hours, during which mother cells proliferate and generate daughter cells (FIGS. 16A-16C). Importantly, the MOMS coating remains confined to mother cells and maintains a high sensor density despite the budding process. Meanwhile, secreted metabolites accumulate within the droplets and are efficiently captured by the MOMS sensors, generating strong fluorescence signals corresponding to the secretion profiles of individual cells.

Step 2: Screening. After incubation, droplets are disrupted to release the yeast cells, and the mother cells are analyzed by flow cytometry. Unlike conventional droplet-based secretion assays, which are typically limited by droplet sorting throughput (~10-200 Hz at a cell density λ of 0.05), the MOMS-based approach bypasses these droplet encapsulation constraints and achieves screening throughputs exceeding 10³-10⁴ Hz, representing a 10-100-fold improvement in processing speed (FIG. 15B).

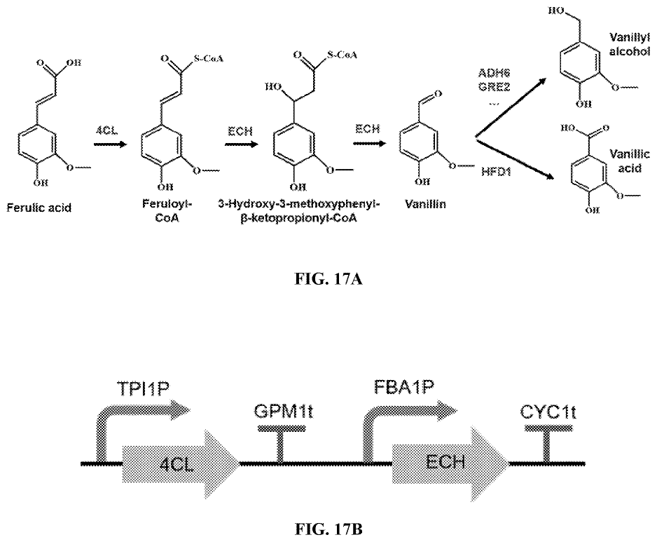

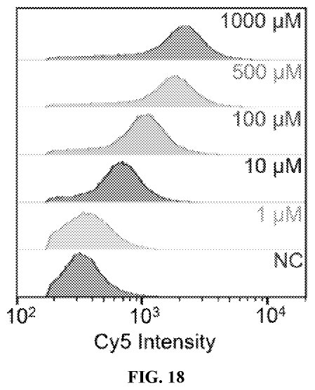

To demonstrate the capability of this platform, engineered Saccharomyces cerevisiae strains are developed for vanillin production. Vanillin synthesis in yeast is catalyzed by the enzymes 4CL and ECH, while native metabolic pathways convert vanillin into vanillyl alcohol or vanillic acid (FIGS. 17A-17B). To enhance vanillin accumulation and secretion, several engineered yeast strains are constructed containing plasmids encoding the 4cl and ech genes. These included VAN-1 (Δgre2), VAN-2 (Δadh6), and VAN-3 (Δadh6 Δgre2), where the deletions partially block vanillin reduction pathways. Bulk fermentation experiments using 1.0 g/L ferulic acid for 48 hours produce vanillin concentrations of 9.6 mg/L for VAN-1, 12.3 mg/L for VAN-2, and 22.9 mg/L for VAN-3. During droplet incubation, fluorescence signal amplification increases from 1.11 to 1.56 for VAN-1, from 1.12 to 1.96 for VAN-2, and from 1.13 to 2.81 for VAN-3 over a 20-hour period (FIG. 15C). Vanillin detection exhibits a wide dynamic range of 1-1000 M after 48 hours of droplet incubation, confirming that the MOMS platform enables accurate quantification without signal saturation during extended cell proliferation (FIG. 18).

In one embodiment, the original ech gene is as stated in SEQ ID NO: 60; and the mutant ech gene with I90N/Y169C/N212Q/P213L amino acid substitutions has a sequence as stated in SEQ ID NO: 61.

To simultaneously analyze vanillin and ATP secretion, yeast cells are coated with two types of MOMS: FITC-labeled MOMS for vanillin and Cy5-labeled MOMS for ATP. Specifically, 1.5 μM vanillin-targeted MOMS and 1.5 μM ATP-targeted MOMS are mixed with 1.5×10⁷ biotinylated yeast cells (WT, VAN-1, VAN-2, or VAN-3) in 1.5 mL DNA streptavidin solution containing 0.5 μM streptavidin, followed by incubation at 30 °C for 30 minutes. For the negative control, WT cells are coated with DNA duplexes labeled with the same fluorescent probes but targeting neither vanillin nor ATP (Table 3). These coated cells (1.5×10⁷) are suspended in 10 mL YPD medium containing 1.0 g/L ferulic acid and encapsulated in water-in-oil droplets using microfluidic droplet generators. The droplets are then incubated at 30 °C for 48 hours, allowing yeast cells to secrete and capture their own vanillin and ATP via MOMS. Following incubation, the droplets are broken, and yeast cells are analyzed via flow cytometry for high-throughput multiplexed single-cell secretion profiling.

High-throughput screening of large populations of single yeast cells (1.2×10⁷ cells) is further demonstrated using co-anchored MOMS containing distinct aptamers for multiplexed detection. Vanillin secretion is detected using FITC fluorescence (excitation: 490 nm; emission: 525 nm), while ATP secretion is simultaneously detected using Cy5 fluorescence (excitation: 646 nm; emission: 664 nm). Flow cytometry analysis clearly distinguishes VAN-3 cells from negative controls (NC), which consist of wild-type (WT) yeast cells coated with DNA duplex sensors that do not target vanillin or ATP (FIG. 15D).

The screening system successfully clusters VAN-1, VAN-2, VAN-3, WT, and NC populations based on their secretion profiles. Among the engineered strains, VAN-3 exhibits the highest vanillin secretion, followed by VAN-2 and VAN-1, all outperforming the WT strain. ATP secretion profiles are similar among the yeast strains but are significantly higher than those of the NC group, further demonstrating the multiplex detection capability of the MOMS platform (FIG. 15E).

In addition, mixtures containing WT and VAN-3 strains at a 1:1 ratio are analyzed by flow cytometry. Three distinct clusters are observed (FIG. 15F): VAN-3 mother cells exhibiting high fluorescence intensity (right cluster), WT mother cells showing quenched fluorescence (middle cluster), and daughter cells displaying low fluorescence (left cluster). When mixtures containing varying WT:VAN-3 ratios (1:2, 1:1, 2:1, 5:1, and 1:0) are analyzed, distinct fluorescence peaks corresponding to each population are observed. This enables precise differentiation and efficient sorting of VAN-3 cells from heterogeneous mixtures (FIG. 15G).

### Example 6. High-Speed Analysis for Rapid Directed Evolution Using MOMS

High-speed sorting of secretory strains for rapid directed evolution is demonstrated using the MOMS platform. In this approach, single-cell secretions are captured by MOMS sensors grafted onto the surfaces of mother yeast cells. After droplet incubation to accumulate secreted metabolites, the droplets are disrupted, and the yeast cells are analyzed directly by flow cytometry. Fluorescence signals generated by MOMS on the cell surfaces are measured at a screening rate of approximately 3.0×10³ cells per second. This method achieves a screening speed more than 30 times faster than conventional droplet-based screening techniques (typically ~10-200 Hz), despite the low droplet encapsulation rate of less than 5%.

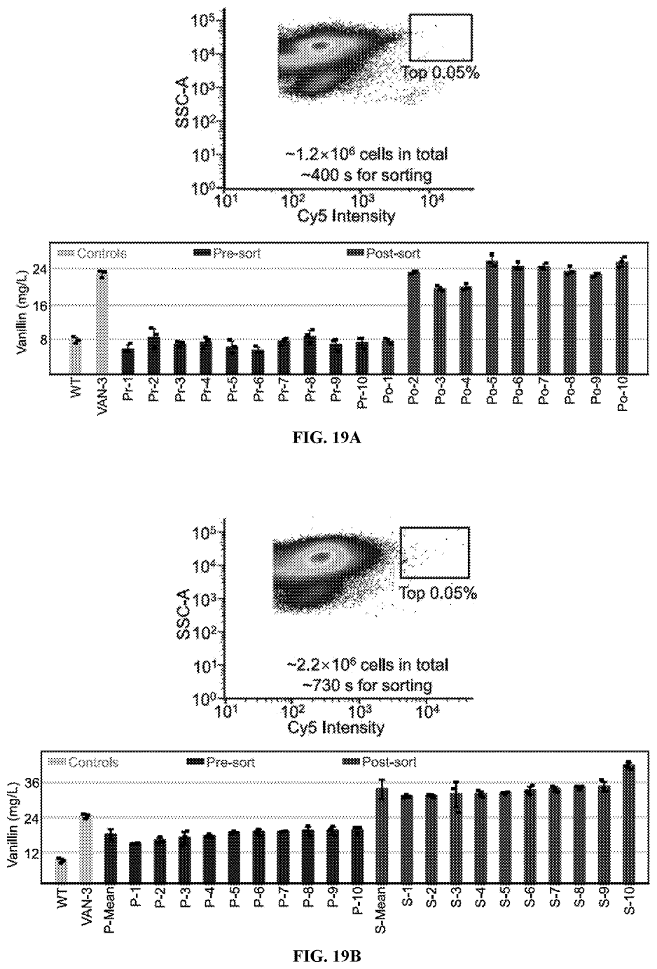

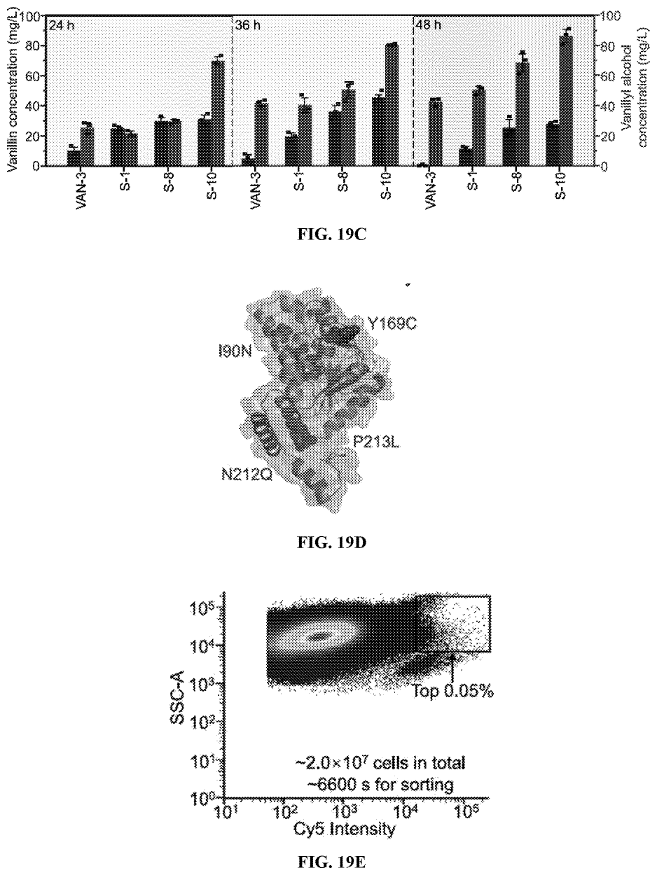

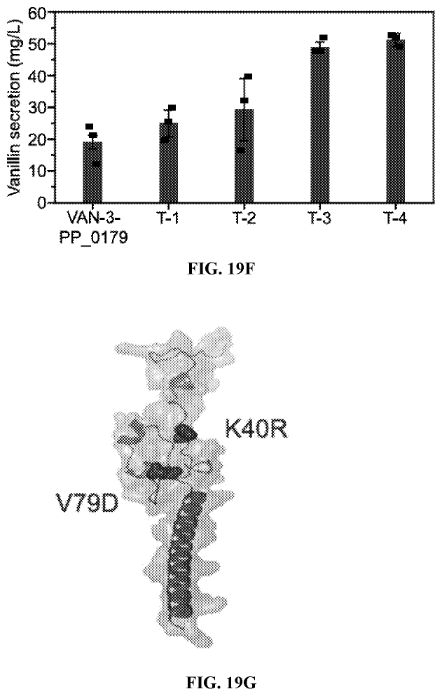

To validate this approach, a mixed population containing 0.5% VAN-3 and 99.5% wild-type (WT) yeast strains is screened to isolate vanillin-secreting cells (FIG. 19A). MOMS-grafted yeast cells are incubated in droplets for 48 hours to capture secreted vanillin. Following droplet disruption, fluorescent yeast cells are sorted by flow cytometry at a rate of approximately 3.0×10³ cells per second. Using this high-speed screening capability, 1.2×10⁶ yeast cells are analyzed and sorted within approximately 7 minutes, successfully isolating approximately 100 vanillin-secreting strains. Vanillin production in randomly selected yeast strains from pre-sorting and post-sorting populations is verified using an absorbance-based assay measuring optical density at 438 nm (OD₄₃₈). After sorting, the secretory VAN-3 population is enriched from 0.5% to approximately 90%, demonstrating high sorting efficiency.

To further demonstrate the application of this strategy in directed evolution, a large mutant library (~2.2×10⁶ variants) is generated using error-prone PCR to evolve the enzyme enoyl-CoA hydratase/aldolase (encoded by the ech gene) for improved vanillin production. The library plasmids are transformed into yeast strain D-3, which contains deletions in the gre2 and adh6 genes, and the resulting cells are coated with vanillin-targeting MOMS sensors. After 48 hours of droplet incubation, the top 0.05% of cells—ranked by fluorescence intensity as an indicator of vanillin secretion—are sorted from a total population of 2.2×10⁶ cells in approximately 12 minutes (FIG. 19B). This high-speed MOMS-assisted sorting process efficiently removes inactive mutants and enriched for high-producing variants.

For culturing and transformation procedures for E. coli and yeast, the strains used herein are listed in Table 4. The construction and amplification of E. coli-S. cerevisiae shuttling plasmids are carried out in E. coli DH5a chemically competent cells. E. coli cultures are grown overnight in Lysogeny Broth (LB) medium (5 g/L yeast extract, 10 g/L tryptone, and 10 g/L sodium chloride) at 37 °C with shaking at 220 rpm. When necessary, ampicillin (100 mg/L) is added for plasmid propagation. E. coli transformations are performed using a standard chemical transformation protocol following the manufacturer's instructions. Transformants are plated on LB agar containing 100 mg/L ampicillin and incubated overnight at 37 °C. Single colonies are inoculated into 5 mL of LB medium and cultured overnight. Plasmids are extracted from E. coli cultures (OD₆₀₀=2.0).

TABLE 4
The strains used in the present invention

| Strains | Description |
|---|---|
| E. coli DH5α | Host for construction of plasmids |
| E. coli Trans1-T1 | Host for construction of mutation library |
| E. coli BL21 (DE3) | Host for protein expression |
| S. cerevisiae BY4742 | Host for vanillin production |
| D-1 | BY4742 derivate, Δgre2 |
| D-2 | BY4742 derivate, Δadh6 |
| D-3 | BY4742 derivate, Δgre2, Δadh6 |
| VAN-1 | D-1 harboring pVAN |
| VAN-2 | D-2 harboring pVAN |
| VAN-1 | D-3 harboring pVAN |
| WT | D-3 harboring pYCP |
| S-1 | D-3 harboring Pvan^{Y75H} |
| S-8 | D-3 harboring Pvan^{D80V} |
| S-10 | D-3 harboring pVAN^{I90N/Y169C/N212Q/P213L} |
| VAN-3-pp_0179 | D-3 harboring pVAN-PP_0179 |
| T-1 | D-3 harboring pVAN-PP_0179^{G45C} |
| T-2 | D-3 harboring pVAN-PP_0179^{K48M} |
| T-3 | D-3 harboring pVAN-PP_0179^{T15I/G45R/Q98H} |
| T-4 | D-3 harboring pVAN-PP_0179^{K40R/V79D} |

For yeast strain development, S. cerevisiae BY4742 is obtained from Beijing Zoman Biotechnology Co., Ltd., China, and propagated in yeast extract peptone dextrose (YPD) medium (10 g/L yeast extract, 20 g/L tryptone, 20 g/L glucose) at 30 °C with shaking at 250 rpm. For plasmid maintenance, yeast strains are cultured in SD medium supplemented with either DO supplement-Leu or DO supplement-Ura. Chemically competent yeast cells are prepared using standard kits. Plasmids are then introduced via chemical transformation, and the engineered yeast cells are plated onto SD agar and incubated at 30 °C for 2-3 days until single colonies formed. These colonies are subsequently cultured overnight in 5 mL of SD medium to generate concentrated cell suspensions (OD₆₀₀=2.0). The molecular profiles of the transformed yeast strains are confirmed by colony PCR, verifying successful plasmid integration.

The plasmids used in the embodiments are listed in Table 5. The E. coli-S. cerevisiae shuttle vectors pYCP (VectorBuilder, China) and pML104 (Addgene, USA) are employed for gene expression and gene deletion in yeast strains, respectively. Recombinant plasmids are constructed using the Gibson assembly method following standard protocols. PCR amplifications are performed using Q5@High Fidelity DNA Polymerase (NEB, USA), with all primers listed in Table 4. For the construction of a plasmid enabling vanillin production in yeast, the target genes 4cl (from Petroselinum crispum) and ech (from Pseudomonas putida KT2440) are PCR amplified using specific primers. The resulting DNA fragments are purified via gel extraction and subcloned into the pYCP vector, generating the recombinant plasmid pVAN. Gene deletions in yeast strain BY4742 are performed using CRISPR/Cas9 gene editing methods.

TABLE 5
The plasmids used in the present invention

| Plasmids | Description |
|---|---|
| pYCP (SEQ ID NO: 62) | E. coli-S. cerevisiae shuttle vector |
| | with Amp^{R} and LEU2 markers |
| pML104 (SEQ ID NO: 63) | E. coli-S. cerevisiae shuttle vector |
| | with Amp^{R} and URA3 markers, |
| | expressing Cas9 and containing single |
| | guide RNA (sgRNA) expression |
| | cassette |
| pET21b (SEQ ID NO: 64) | Vector for expression of recombinant |
| | protein in E. coli, harboring Amp^{R} |
| | marker |
| pML104-adh6 (SEQ ID NO: 65) | pML104 derivate, containing the |
| | sgRNA for adh6 gene deletion |
| pML104-gre2 (SEQ ID NO: 66) | pML104 derivate, containing the |
| | sgRNA for gre2 gene deletion |
| pVAN (SEQ ID NO: 67) | pYCP derivate, containing the |
| | expression cassettes for 4cl and ech |
| | genes: P_{TPI1}-4cl-T_{GPM1}-P_{FBA1}-ech-T_{CYC1} |
| pVAN^{D80V} (SEQ ID NO: 68) | pVAN derivate, with mutation site |
| | D80V in ech gene |
| pVAN^{Y75H} (SEQ ID NO: 69) | pVAN derivate, with mutation site |
| | Y75H in ech gene |
| pVAN^{I90N/Y169C/N212Q/P213L} (SEQ ID | pVAN derivate, with mutation sites |
| NO: 70) | I90N/Y169C/N212Q/P213L in ech |
| | gene |
| pECH (SEQ ID NO: 71) | pET21b derivate, expressing ech gene |
| Pech^{Y75H} (SEQ ID NO: 72) | pECH derivate, with mutation site |
| | Y75H in ech gene |
| Pech^{D80V} (SEQ ID NO: 73) | pECH derivate, with mutation site |
| | D80V in ech gene |
| pECH^{I90N/Y169C/N212Q/P213L} (SEQ ID | pECH derivate, with mutation sites |
| NO: 74) | I90N/Y169C/N212Q/P213L in ech |
| | gene |
| pVAN-PP_0179 (SEQ ID NO: 75) | pVAN derivate, containing the |
| | expression cassettes for 4cl, ech and |
| | pp_0179 genes: P_{TPI1}-4cl-T_{GPM1}-P_{FBA1}- |
| | ech-T_{CYC1}- P_{TPI1}-pp_0179-T_{GPM1} |
| pVAN-PP_0179^{G45C} (SEQ ID NO: 76) | pVAN-PP_0179 derivate, with |
| | mutation site G45C in pp_0179 gene |
| pVAN-PP_0179^{K48M} (SEQ ID NO: 77) | pVAN-PP_0179 derivate, with |
| | mutation site K48M in pp_0179 gene |
| pVAN-P_0179^{T15I/G45R/Q98H} (SEQ ID | pVAN-PP_0179 derivate, with |
| NO: 78) | mutation sites T15I/G45R/Q98H in |
| | pp_0179 gene |
| pVAN-P_0179^{K40R/V79D} (SEQ ID NO: | pVAN-PP_0179 derivate, with |
| 79) | mutation sites K40R/V79D in pp_0179 |
| | gene |

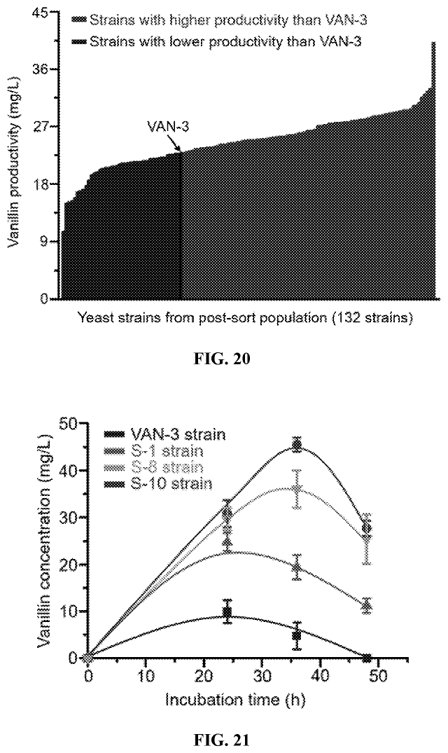

Following sorting, approximately 301 candidate strains are isolated and cultured on SD agar plates. From these, 132 clones are randomly selected and cultured individually to verify vanillin production using the absorbance-based assay. The results indicate that the sorted pool achieves over 68% accuracy in identifying rare secretory phenotypes (FIG. 20). Among these strains, the top 10 variants ranked by vanillin production exhibit an average production level approximately 1.93-fold higher than that of variants randomly selected from the unsorted parent population.

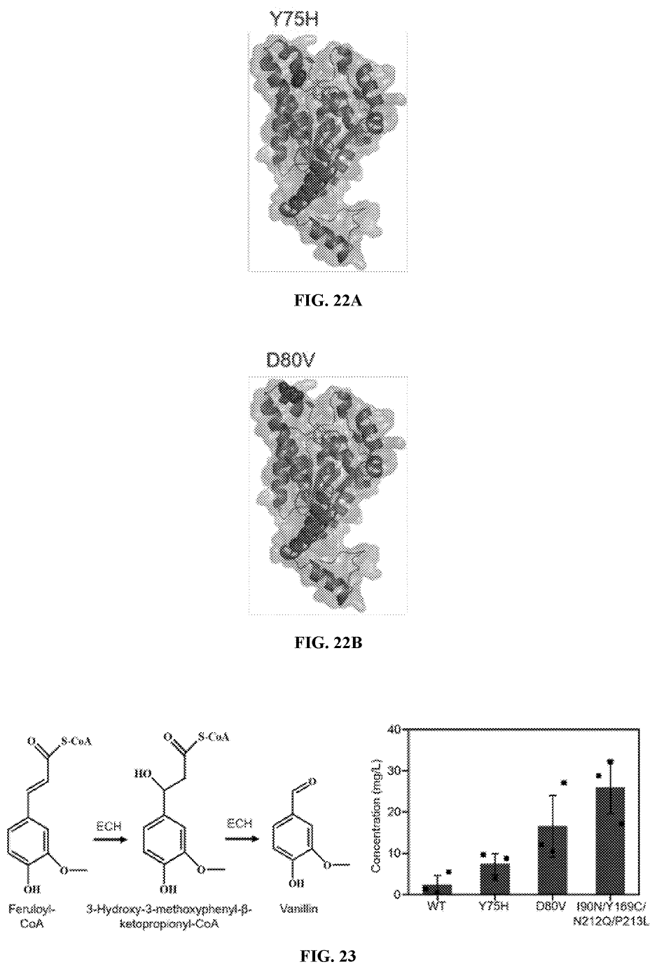

The vanillin productivity of the sorted strains is further validated by batch fermentation analysis using high-performance liquid chromatography (HPLC) (FIG. 19C). Among the variants, strain S-10 exhibits the highest yield, producing 86.4 mg/L vanillyl alcohol and 27.7 mg/L vanillin (corresponding to a total of 113.0 mg/L vanillin equivalents) after 48 hours. This represents a 2.7-fold increase compared with the control strain VAN-3, which produces 42.6 mg/L vanillyl alcohol (equivalent to 42.0 mg/L vanillin). Notably, VAN-3 secretes detectable vanillin during the first 24 hours; however, the vanillin concentration declines significantly after 36 hours, likely due to conversion to vanillyl alcohol, and becomes nearly undetectable after 48 hours when vanillyl alcohol levels reach 42.6 mg/L. A time-course comparison among strains is shown in FIG. 21.

Similar vanillin depletion patterns are observed in ECH mutant strains S-1, S-8, and S-10, although the rate of decline varies among strains. This observation suggests that vanillin is converted into vanillyl alcohol by endogenous enzymes such as alcohol dehydrogenases (ADHs), aldoketo reductases (AKRs), and aldehyde reductases (ALDRs). Sequencing analysis of the vanillin-secreting variants reveals three unique full-length active ech sequences without internal stop codons. Compared with the original ECH enzyme, the ECH variant identified in strain S-10 contains four mutations (I90N, Y169C, N212Q, and P213L) (FIG. 19D). Additional mutations, including D80V and Y75H, are identified in the ECH variants from strains S-8 and S-1, respectively (FIGS. 22A-22B).

To evaluate enzyme activity, wild-type and mutant ech genes are expressed in Escherichia coli BL21 (DE3). The cultures are incubated with 200 μM feruloyl-CoA and 7.5 M 3-NAD at 37 °C for 24 hours, after which vanillin concentrations in the supernatant are quantified using an absorbance-based assay. All three mutant enzymes produce significantly higher levels of vanillin compared with the wild-type enzyme. In particular, the I90N/Y169C/N212Q/P213L variant yields the highest vanillin production (~26 mg/L), corresponding to approximately an 11-fold increase over the wild type (FIG. 23).

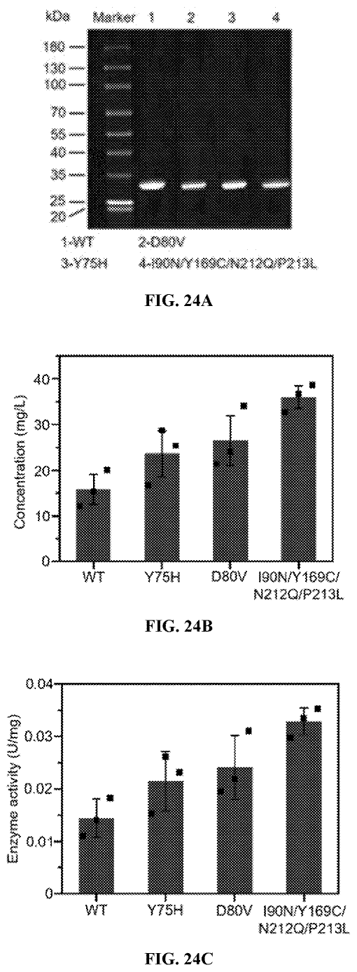

To further quantify catalytic efficiency, both wild-type and mutant ECH enzymes are purified using nickel-affinity chromatography (FIG. 24A). Equal amounts of purified enzyme (1 mg/mL) are incubated with 300 μM feruloyl-CoA and 7.5 μM 3-NAD at 30 °C for 12 hours. Vanillin production is measured using the same absorbance-based method (FIG. 24B), and relative enzyme activities are calculated accordingly (FIG. 24C). All mutant enzymes consistently exhibit higher catalytic activity than the wild type, with the I90N/Y169C/N212Q/P213L variant displaying an approximately 2.3-fold increase in enzymatic activity, confirming improved catalytic performance.

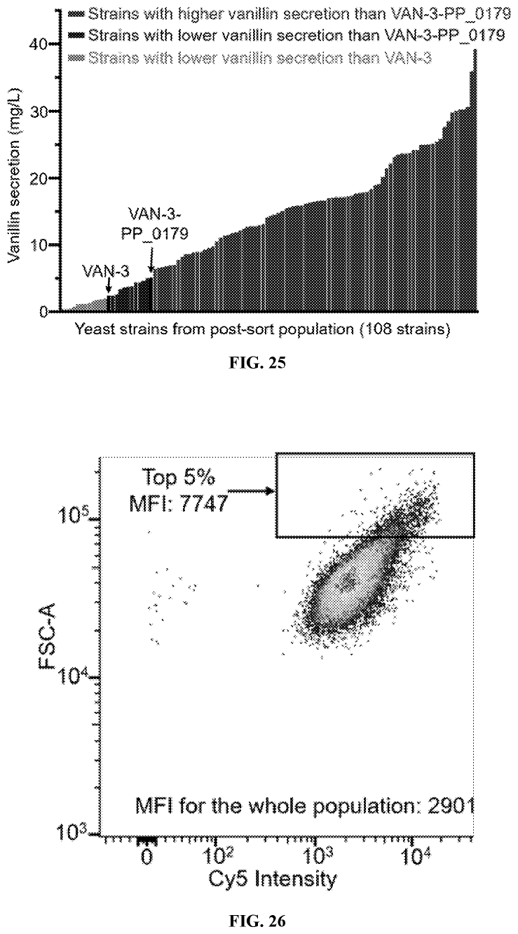

In addition to evolving ECH enzymes, the MOMS platform is further applied to engineer transporter proteins that enhance extracellular secretion in yeast. A mutant library of the vanillin transporter PP_0179 is generated in the VAN-3 strain using error-prone PCR, yielding more than 2×10⁷ variants. Using the MOMS platform, single-cell screening is performed by capturing and quantifying vanillin secreted from individual mutants. The top 0.05% of high-secreting strains are sorted (FIG. 19E), plated, and 108 colonies are randomly selected for validation (FIG. 25). Among these isolates, more than 78% demonstrate improved performance compared with the wild-type PP_0179 transporter, confirming the high precision of the screening platform.

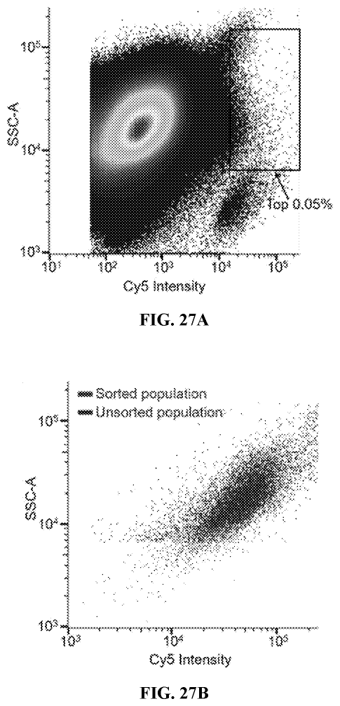

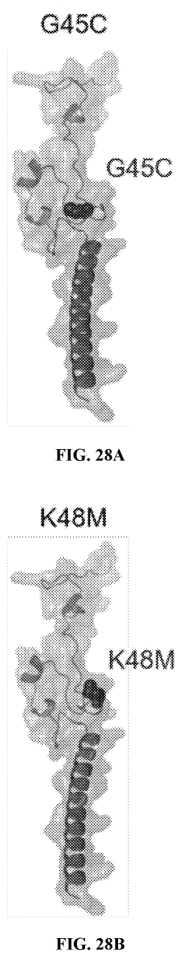

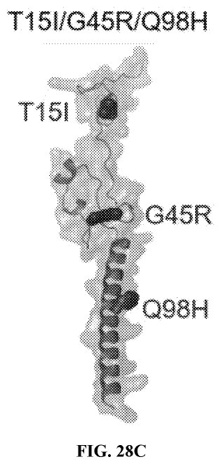

Potential fluorescence signal variations caused by differences in cell size or budding (FIG. 26) are found to have minimal impact on sorting accuracy. A stringent gating strategy (FIG. 27A) ensures consistent cell size distributions before and after sorting (FIG. 27B). The ten best-performing variants are subsequently evaluated through batch fermentation experiments, with four variants exhibiting significantly enhance vanillin secretion (FIG. 19F). The most effective variant, derived from the T-4 strain, contains K40R and V79D mutations that show a synergistic enhancement of transporter efficiency (FIG. 19G). Additional key mutations—including G45C in the T-1 strain, K48M in the T-2 strain, and the combined mutations T15I/G45R/Q98H in the T-3 strain—are presented in FIGS. 28A-28C. These results highlight the versatility of the MOMS platform for practical applications in metabolic engineering, including the directed evolution of transporter proteins.

### Example 7. High-Flexibility of MOMS for Protein Analysis

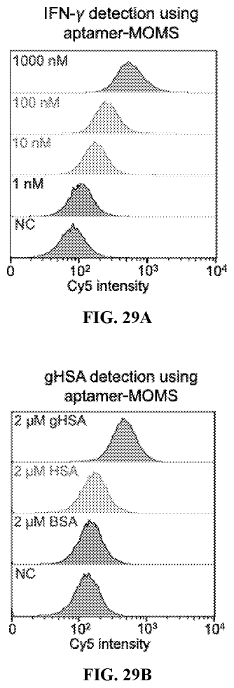

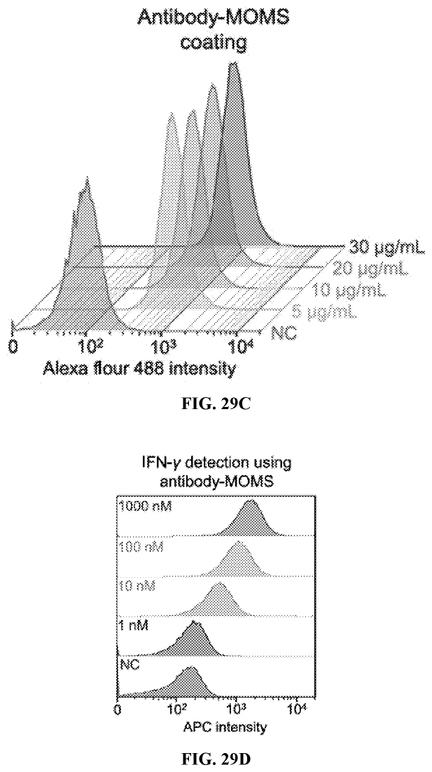

MOMS is a highly versatile platform capable of accommodating a wide range of sensor types through biotin-streptavidin interactions, enabling the detection of diverse molecular targets. The system is compatible with high-affinity aptamers for both metabolite detection (Table. 6) and protein detection (Table. 7). For example, using aptamers targeting human interferon gamma (IFN-γ), the aptamer-based MOMS system (aptamer-MOMS) can detect IFN-γ across a dynamic range of 1-1000 nM with a limit of detection (LOD) of approximately 1 nM (FIG. 29A). Similarly, glycosylated human serum albumin (gHSA)-specific aptamers enable aptamer-MOMS to selectively recognize glycosylated pharmaceutical proteins (FIG. 29B). For molecular targets lacking validated aptamers, emerging structure-switching aptamer technologies provide promising alternatives for expanding the sensing capability of the MOMS platform.

In addition to aptamer-based sensing, the MOMS platform can be adapted to an antibody-based format (antibody-MOMS) by coating the cell surface with streptavidin-conjugated antibodies. This configuration enables the detection of a broader range of protein targets. For instance, yeast cells coated with streptavidin-modified IFN-γ antibodies achieve uniform surface coverage at antibody concentrations above 5 g/mL (FIG. 29C), enabling sensitive detection with a limit of detection of approximately 1 nM and a wide dynamic range of 1-1000 nM (FIG. 29D). This modular architecture allows MOMS to be readily customized for detecting various analytes—from small molecules to large proteins—simply by replacing the sensing element.

TABLE 6
Aptamers for metabolite analysis

| Target | DNA aptamer sequence |
|---|---|
| ATP | TACCTGGGGGAGTATTGCGGAGGAAGGT (SEQ ID NO: |
| | 80) |
| Glucose | CTCTCGGGACGACCGTGTGTGTTGCTCTGTAACAGT |
| | GTCCATTGTCGTCCC (SEQ ID NO: 81) |
| Vanillin | CAGGAGAAACATGGAGTCTCGATGAT (SEQ ID NO: 82) |
| Naringenin | CTAGCAGTIGTGGTAGAGTCACCACAA (SEQ ID NO: |
| | 83) |
| Arginine | CCACCAACGTGTCGCCTCGTCG (SEQ ID NO: 84) |
| L-tyrosine | CTCTCGGGACGACCTCTCGGGACGACGGCCCGATCT |
| | CAGAGTAGTCGTCCCGTCGTCCC (SEQ ID NO: 85) |
| Phenylalanine | CTCTCGGGACGACCGCGTTTCCCAAGAAAGCAAGTA |
| | TTGGTTGGTCGTCCC (SEQ ID NO: 86) |
| L-Lactate | GACTAGCGCGTATGAATGCTTTTCTATGGA (SEQ ID |
| | NO: 87) |
| L-tryptophan | AGCACGTTGGTTAGGTCAGGTTTGGGTTTCGTGC |
| | (SEQ ID NO: 88) |
| Testosterone | CTCTCGGGACGACGGGATGTCCGGGGTACGGTGGTT |
| | GCAGTTCGTCGTCCC (SEQ ID NO: 89) |
| Hydrocortisone | CTCTCGGGACGACGCCCGCATGTTCCATGGATAGTCT |
| | TGACTAGTCGTCCC (SEQ ID NO: 90) |
| Aldosterone | CTCTCGGGACGACAGATAGTTGTTCTTAGCGATGTTC |
| | AGCGTTGTCGTCCC (SEQ ID NO: 91) |

TABLE 7
Aptamers for protein analysis

| Target | DNA aptamer sequence |
|---|---|
| Human IFN-γ | TGGGGTTGGTTGTGTTGGGTGTTGTGT (SEQ ID NO: |
| | 92) |
| glycosylated HSA | GGTGCGGTTCGTGCGGTTGTAGTACTCGTGGCCG |
| | (SEQ ID NO: 93) |
| Human TNF-α | TGGTGGATGGCGCAGTCGGCGACAA (SEQ ID NO: 94) |
| Human insulin | GGTGGTGGGGGGGGTTGGTAGGGTGTCTTC (SEQ ID |
| | NO: 95) |
| Human IL-2 | TCGTTCGGGACTGCTCGGGATTGCGGATA (SEQ ID |
| | NO: 96) |
| α-defensin human | GGCAGGAAGACAAACACGGGGCGGCCGAAACAATG |
| neutrophil protein 1 | CGTTTCGGACCCAGCAACTCGTGGTCTGTGGTGCTG |
| | T (SEQ ID NO: 97) |
| PD-L1 | TACAGGTTCTGGGGGGTGGGTGGGGAACCTGTT |
| | (SEQ ID NO: 98) |
| Thrombin | CCTGCCACGCTCCGCT (SEQ ID NO: 99) |
| Salivary α-amylase | TGAACGACGTGAATAGTGTTTGTGGGTCCG (SEQ ID |
| | NO: 100) |
| Streptavidin | CTATACTCCACTTTGCTATTTCTCGGTTCCTTCACGCG |
| | CCGATCGCAGGCTGATGAATTG (SEQ ID NO: 101) |

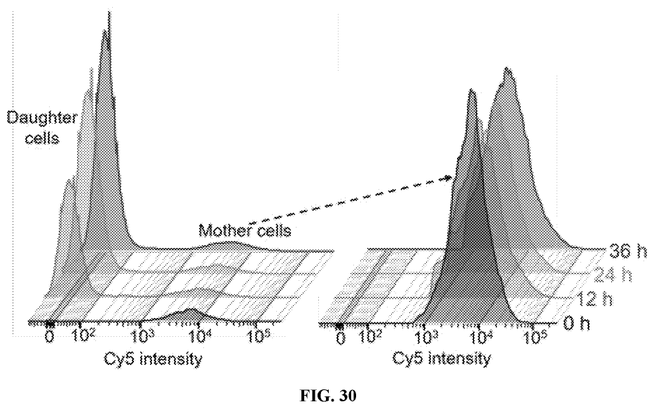

Notably, beyond Saccharomyces cerevisiae, the MOMS platform can also be adapted to other budding fungi, such as Pichia pastoris, for secretion profiling applications (FIG. 30). A key advantage of the MOMS strategy lies in its reliance on the asymmetric budding process of yeast, which ensures that surface-bound sensors remain localized on mother cells during cell division. This mechanism preserves a stable sensor density across generations and enables robust fluorescence-based detection of extracellular secretions. In contrast, symmetrically dividing organisms such as Escherichia coli distribute surface-bound sensors evenly between daughter cells, resulting in signal dilution and reduced detection stability, thereby making them unsuitable for MOMS-based sensing. Table 8 summarizes fungal species that are compatible or incompatible with the MOMS platform.

TABLE 8
The fungal species for MOMS.

| | |
|---|---|
| Representative fungal species | Saccharomyces cerevisiae; |
| compatible with MOMS | Pichia pastoris; |
| | Yarrowia lipolytica; |
| | Phaffia rhodozyma; |
| | Hansenula polymorpha; |
| | Kluyveromyces lactis; |
| | Cryptoccus neoformans; |
| | Candida albicans. |
| Representative fungal species | Schizosaccharomyces; |
| incompatible with MOMS | Filamentous fungi; |
| | Dimorphic fungi. |

In summary, the present invention provides a molecular sensing platform, referred to as MOMS, which selectively anchors molecular sensors onto mother yeast cells without transfer to daughter cells during budding. This selective localization enables high-density sensor immobilization (approximately 1.4×10⁷ sensors per cell) on the surface of mother yeast cells, while maintaining functionality over multiple cell divisions for up to three days.

The MOMS platform offers three key advantages. First, high sensitivity is achieved through dense sensor coverage and minimal diffusion distance between the sensors and the cell surface, enabling highly sensitive detection with limits of detection below 1 μM. Second, high throughput is enabled by a two-step process: MOMS-coated yeast cells are initially encapsulated in droplets to capture secreted molecules, generating fluorescence signals on the mother cell surface, followed by droplet disruption and flow cytometric analysis, allowing high-throughput phenotyping of more than 10⁷ yeast cells. Third, high speed is achieved by directly sorting yeast cells without requiring droplet encapsulation during analysis, thereby overcoming the low single-cell encapsulation efficiency (~1-10%) associated with conventional droplet-based methods and enabling sorting speeds more than 30-fold higher.

To validate the platform, a mutant library comprising approximately 10⁶ variants derived from the parental strain VAN-3 is screened. The top 0.05% of mother yeast cells exhibiting the highest vanillin secretion are isolated via high-speed sorting. The selected population demonstrates a plateau in secretion activity at approximately 2.7-fold higher than that of the parental strain. Sequencing analysis identifies four unique variants, including mutations in the ech gene that resulted in amino acid substitutions. Among these, the S-10 strain, harboring mutations I90N, Y169C, N212Q, and P213L, exhibits the highest vanillin productivity and strong potential for directed evolution.

Further improvements may be achieved by combining disruption of endogenous vanillin degradation pathways, including ADHs, AKRs, and ALDRs, with iterative MOMS-based directed evolution targeting key enzymes such as ECH and 4CL, thereby enabling large-scale vanillin production at industrially relevant titers (e.g., >2.5 g/L).

Notably, the MOMS platform is highly versatile and supports a wide range of metabolic engineering applications. It can be applied to the directed evolution of extracellular transport proteins, such as PP_0179, and can also be used for detecting extracellular proteins, including IFN-γ and glycosylated proteins. The platform further enables the isolation of high-producing strains for subsequent optimization, and may be extended to analyze protein heterogeneity, such as immunoglobulins with different glycosylation profiles.

Accordingly, MOMS represents an advanced analytical tool for sensitive, high-throughput screening and rapid sorting of single-cell yeast secretions, enabling efficient identification of rare high-performing strains within large populations and providing new opportunities for metabolic analysis and strain engineering.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A method for ultra-high-throughput screening of yeast metabolism, comprising:
providing a population of yeast cells;
anchoring a plurality of molecular sensors on membrane surfaces of mother yeast cells to form molecular sensors on membrane surfaces of mother cells (MOMS), wherein the molecular sensors are selectively retained on the mother yeast cells during budding and are not transferred to daughter cells;
incubating the yeast cells in conditions allowing secretion of metabolic products from individual cell of the yeast cells;
capturing the secreted metabolic products using the molecular sensors anchored on the membrane surfaces of the mother yeast cells;
generating detectable signals from the molecular sensors upon interaction with the captured metabolic products; and
screening the yeast cells based on the detectable signals to identify yeast cells exhibiting a desired metabolic secretion phenotype;
wherein the secreted metabolic products comprise target metabolites and proteins.

2. The method of claim 1, wherein the secreted metabolic products comprise target metabolites and proteins, and the molecular sensors comprise nucleic acid aptamer sensors configured to bind to the target metabolites and proteins.

3. The method of claim 2, wherein the aptamer sensors are immobilized on the membrane surfaces of the mother yeast cells via a biotin-streptavidin interaction.

4. The method of claim 1, wherein the molecular sensors are immobilized at a density of at least 10⁶ sensors per yeast cell.

5. The method of claim 1, wherein the molecular sensors generate fluorescence signals upon binding to the secreted metabolic products.

6. The method of claim 1, further comprising encapsulating individual MOMS-coated yeast cells in microfluidic droplets to capture secreted metabolites within the droplets.

7. The method of claim 6, further comprising disrupting the droplets after incubation to release the yeast cells for subsequent screening.

8. The method of claim 1, wherein the molecular sensors comprise structure-switching aptamers that emit fluorescence upon binding to a target molecule.

9. The method of claim 1, wherein the target metabolites comprise vanillin, ATP, glucose, and zinc ions.

10. The method of claim 1, wherein the target proteins comprise interferon-gamma (IFN-γ) and glycosylated human serum albumin.

11. The method of claim 1, wherein the yeast cells comprise Saccharomyces cerevisiae or Pichia pastoris.

12. The method of claim 1, further comprising generating a mutant yeast library and performing directed evolution by selecting yeast variants exhibiting enhanced secretion phenotypes.

13. The method of claim 1, further comprising performing multiplex detection of multiple metabolites by anchoring different molecular sensors targeting different molecules on the membrane surface of the mother yeast cells.

14. Functionalized yeast cells for detecting extracellular secretions, comprising:
at least one mother yeast cell having a membrane surface; and
one or more molecular sensors immobilized on the membrane surface of the mother yeast cell;
wherein the molecular sensors are configured to bind secreted molecules produced by the functionalized yeast cells and generate detectable signals; and
wherein the molecular sensors remain localized on the mother yeast cell during budding and are substantially not transferred to any daughter cell.

15. The functionalized yeast cell of claim 14, wherein the molecular sensors comprise aptamer sensors.

16. The functionalized yeast cell of claim 14, wherein the molecular sensors are immobilized through biotin-streptavidin binding.

17. The functionalized yeast cell of claim 14, wherein the molecular sensors comprise fluorescence-based structure-switching aptamers.

18. A system for ultra-high-throughput screening of microbial secretions, comprising:
the functionalized yeast cells of claim 14;
a droplet microfluidic device configured to encapsulate individual cell of the functionalized yeast cells in droplets;
an incubation module configured to allow secretion and capture of metabolites by the molecular sensors; and
a detection and sorting device configured to detect signals generated by the molecular sensors and to isolate yeast cells exhibiting desired secretion phenotypes;
wherein the system is configured to screen at least 10⁷ yeast cells in one cycle; and
wherein the system is configured to sort yeast cells at a rate of at least 10³ cells per second.

19. The system of claim 18, wherein the molecular sensors comprise aptamer sensors configured to bind secreted metabolites.

20. A mutant enoyl-CoA hydratase/aldolase (ECH) enzyme comprising at least one amino acid substitution selected from I90N, Y169C, and N212Q relative to a wild-type ECH enzyme.


SEQUENCE LISTING (101 sequences)

SEQ ID NO: 1 - DNA, 40 bp, synthetic construct
  source 1..40: mol_type = other DNA
  misc_feature 1..40: note = forward primer for ADH6-sgRNA
gatcgacattaagatcgaagcatgtgggttttagagctag

SEQ ID NO: 2 - DNA, 36 bp, synthetic construct
  source 1..36: mol_type = other DNA
  misc_feature 1..36: note = reverse primer for ADH6-sgRNA
ctagctctaaaacccacatgcttcgatcttaatgtc

SEQ ID NO: 3 - DNA, 20 bp, synthetic construct
  source 1..20: mol_type = other DNA
  misc_feature 1..20: note = forward primer for ADH6-donorU
ggaccttgacgtggaattcc

SEQ ID NO: 4 - DNA, 22 bp, synthetic construct
  source 1..22: mol_type = other DNA
  misc_feature 1..22: note = reverse primer for ADH6-donorU
ggcttttcttgttgttgtgttg

SEQ ID NO: 5 - DNA, 23 bp, synthetic construct
  source 1..23: mol_type = other DNA
  misc_feature 1..23: note = forward primer for ADH6-donorD
cagactaggttgtcaagctcttg

SEQ ID NO: 6 - DNA, 20 bp, synthetic construct
  source 1..20: mol_type = other DNA
  misc_feature 1..20: note = reverse primer for ADH6-donorD
catggaacatcctcatccgc

SEQ ID NO: 7 - DNA, 37 bp, synthetic construct
  source 1..37: mol_type = other DNA
  misc_feature 1..37: note = forward primer for GRE2-sgRNA
gatcaaaggccgagaatttaacgggttttagagctag

SEQ ID NO: 8 - DNA, 33 bp, synthetic construct
  source 1..33: mol_type = other DNA
  misc_feature 1..33: note = reverse primer for GRE2-sgRNA
ctagctctaaaacccgttaaattctcggccttt

SEQ ID NO: 9 - DNA, 23 bp, synthetic construct
  source 1..23: mol_type = other DNA
  misc_feature 1..23: note = forward primer for GRE2-donorU
tgtacgctatagtttcctttcaa

SEQ ID NO: 10 - DNA, 20 bp, synthetic construct
  source 1..20: mol_type = other DNA
  misc_feature 1..20: note = reverse primer for GRE2-donorU
tttacgggcgtgtgatactg

SEQ ID NO: 11 - DNA, 20 bp, synthetic construct
  source 1..20: mol_type = other DNA
  misc_feature 1..20: note = forward primer for GRE2-donorD
tcgtccttcttgaagtccca

SEQ ID NO: 12 - DNA, 20 bp, synthetic construct
  source 1..20: mol_type = other DNA
  misc_feature 1..20: note = reverse primer for GRE2-donorD
cgacactgcctcccaaattt

SEQ ID NO: 13 - DNA, 40 bp, synthetic construct
  source 1..40: mol_type = other DNA
  misc_feature 1..40: note = forward primer for PTPI1
aactttgtatagaaaagttgaaggatgagccaagaataag

SEQ ID NO: 14 - DNA, 38 bp, synthetic construct
  source 1..38: mol_type = other DNA
  misc_feature 1..38: note = reverse primer for PTPI1
tacaaacttgttttagtttatgtatgtgttttttgtag

SEQ ID NO: 15 - DNA, 31 bp, synthetic construct
  source 1..31: mol_type = other DNA
  misc_feature 1..31: note = forward primer for 4CL
taaactaaaacaagtttgtacaaaaaagcag

SEQ ID NO: 16 - DNA, 28 bp, synthetic construct
  source 1..28: mol_type = other DNA
  misc_feature 1..28: note = reverse primer for 4CL
ttcttcagacttattttggaaggtcccc

SEQ ID NO: 17 - DNA, 31 bp, synthetic construct
  source 1..31: mol_type = other DNA
  misc_feature 1..31: note = forward primer for TGPM1
tccaaaataagtctgaagaatgaatgatttg

SEQ ID NO: 18 - DNA, 32 bp, synthetic construct
  source 1..32: mol_type = other DNA
  misc_feature 1..32: note = reverse primer for TGPM1
cagttggatctattgctataacatgtcatgtc

SEQ ID NO: 19 - DNA, 29 bp, synthetic construct
  source 1..29: mol_type = other DNA
  misc_feature 1..29: note = forward primer for PFBA1
tatagcaatagatccaactggcaccgctg

SEQ ID NO: 20 - DNA, 58 bp, synthetic construct
  source 1..58: mol_type = other DNA
  misc_feature 1..58: note = reverse primer for PFBA1
atttgctcattttgaatatgtattacttggttatggttatatatgacaaaagaaaaag

SEQ ID NO: 21 - DNA, 29 bp, synthetic construct
  source 1..29: mol_type = other DNA
  misc_feature 1..29: note = forward primer for ECH
catattcaaaatgagcaaatacgaaggac

SEQ ID NO: 22 - DNA, 38 bp, synthetic construct
  source 1..38: mol_type = other DNA
  misc_feature 1..38: note = reverse primer for ECH
tttgtacaagaaagctgggttcacctcttgtaagcctg

SEQ ID NO: 23 - DNA, 21 bp, synthetic construct
  source 1..21: mol_type = other DNA
  misc_feature 1..21: note = forward primer for pYCP
acccagctttcttgtacaaag

SEQ ID NO: 24 - DNA, 24 bp, synthetic construct
  source 1..24: mol_type = other DNA
  misc_feature 1..24: note = reverse primer for pYCP
caacttttctatacaaagttggtg

SEQ ID NO: 25 - DNA, 22 bp, synthetic construct
  source 1..22: mol_type = other DNA
  misc_feature 1..22: note = forward primer for 4CL-colony
tgagagtgagatgcctgaagtt

SEQ ID NO: 26 - DNA, 22 bp, synthetic construct
  source 1..22: mol_type = other DNA
  misc_feature 1..22: note = reverse primer for 4CL-colony
attgctttatctcctcctccgt

SEQ ID NO: 27 - DNA, 21 bp, synthetic construct
  source 1..21: mol_type = other DNA
  misc_feature 1..21: note = forward primer for ECH-colony
ttttccttcttcttcgcccac

SEQ ID NO: 28 - DNA, 21 bp, synthetic construct
  source 1..21: mol_type = other DNA
  misc_feature 1..21: note = reverse primer for ECH-colony
tagtcctcgttctgttcccac

SEQ ID NO: 29 - DNA, 30 bp, synthetic construct
  source 1..30: mol_type = other DNA
  misc_feature 1..30: note = forward primer for epECH
gttcttccttgcgttattcttctgttcttc

SEQ ID NO: 30 - DNA, 27 bp, synthetic construct
  source 1..27: mol_type = other DNA
  misc_feature 1..27: note = reverse primer for epECH
cactttgtacaagaaagctgggttcac

SEQ ID NO: 31 - DNA, 27 bp, synthetic construct
  source 1..27: mol_type = other DNA
  misc_feature 1..27: note = forward primer for pYCP-L
gtgaacccagctttcttgtacaaagtg

SEQ ID NO: 32 - DNA, 30 bp, synthetic construct
  source 1..30: mol_type = other DNA
  misc_feature 1..30: note = reverse primer for pYCP-L
gaagaacagaagaataacgcaaggaagaac

SEQ ID NO: 33 - DNA, 50 bp, synthetic construct
  source 1..50: mol_type = other DNA
  misc_feature 1..50: note = forward primer for ECH
ttaactttaagaaggagatatacatatgagcaaatacgaaggacgttgga

SEQ ID NO: 34 - DNA, 52 bp, synthetic construct
  source 1..52: mol_type = other DNA
  misc_feature 1..52: note = reverse primer for ECH
ggatctcagtggtggtggtggtggtgcctcttgtaagcctgcaatccaggtt

SEQ ID NO: 35 - DNA, 52 bp, synthetic construct
  source 1..52: mol_type = other DNA
  misc_feature 1..52: note = forward primer for pET21b-ECH
aacctggattgcaggcttacaagaggcaccaccaccaccaccactgagatcc

SEQ ID NO: 36 - DNA, 50 bp, synthetic construct
  source 1..50: mol_type = other DNA
  misc_feature 1..50: note = reverse primer for pET21b-ECH
tccaacgtccttcgtatttgctcatatgtatatctccttcttaaagttaa

SEQ ID NO: 37 - DNA, 27 bp, synthetic construct
  source 1..27: mol_type = other DNA
  misc_feature 1..27: note = forward primer for pVAN-GJ
gcggccctgcattaatgaatcggccaa

SEQ ID NO: 38 - DNA, 28 bp, synthetic construct
  source 1..28: mol_type = other DNA
  misc_feature 1..28: note = reverse primer for pVAN-GJ
agcttgcaaattaaagccttcgagcgtc

SEQ ID NO: 39 - DNA, 58 bp, synthetic construct
  source 1..58: mol_type = other DNA
  misc_feature 1..58: note = forward primer for PTPI1
ttgggacgctcgaaggctttaatttgcaagctaaggatgagccaagaataagggaaca

SEQ ID NO: 40 - DNA, 34 bp, synthetic construct
  source 1..34: mol_type = other DNA
  misc_feature 1..34: note = reverse primer for PTPI1
ttttagtttatgtatgtgttttttgtagttatag

SEQ ID NO: 41 - DNA, 59 bp, synthetic construct
  source 1..59: mol_type = other DNA
  misc_feature 1..59: note = forward primer for PP_0179
aactacaaaaaacacatacataaactaaaaatggttcaaagattgactttcagaagacg

SEQ ID NO: 42 - DNA, 58 bp, synthetic construct
  source 1..58: mol_type = other DNA
  misc_feature 1..58: note = reverse primer for PP_0179
aatcatcaaatcattcattcttcagacttacttagcagctttacgaccagccttggca

SEQ ID NO: 43 - DNA, 25 bp, synthetic construct
  source 1..25: mol_type = other DNA
  misc_feature 1..25: note = forward primer for TGPM1
gtctgaagaatgaatgatttgatga

SEQ ID NO: 44 - DNA, 58 bp, synthetic construct
  source 1..58: mol_type = other DNA
  misc_feature 1..58: note = reverse primer for TGPM1
gcgttggccgattcattaatgcagggccgctattgctataacatgtcatgtcaccatt

SEQ ID NO: 45 - DNA, 34 bp, synthetic construct
  source 1..34: mol_type = other DNA
  misc_feature 1..34: note = forward primer for epPP_0179
taactacaaaaaacacatacataaactaaaaatg

SEQ ID NO: 46 - DNA, 32 bp, synthetic construct
  source 1..32: mol_type = other DNA
  misc_feature 1..32: note = reverse primer for epPP_0179
gaaatcatcaaatcattcattcttcagactta

SEQ ID NO: 47 - DNA, 28 bp, synthetic construct
  source 1..28: mol_type = other DNA
  misc_feature 1..28: note = ATP aptamer
tacctgggggagtattgcggaggaaggt

SEQ ID NO: 48 - DNA, 12 bp, synthetic construct
  source 1..12: mol_type = other DNA
  misc_feature 1..12: note = Quenching cDNA to ATP aptamer
ctcccccaggta

SEQ ID NO: 49 - DNA, 51 bp, synthetic construct
  source 1..51: mol_type = other DNA
  misc_feature 1..51: note = D-glucose aptamer
ctctcgggacgaccgtgtgtgttgctctgtaacagtgtccattgtcgtccc

SEQ ID NO: 50 - DNA, 13 bp, synthetic construct
  source 1..13: mol_type = other DNA
  misc_feature 1..13: note = Quenching cDNA to D-glucose aptamer
gtcgtcccgagag

SEQ ID NO: 51 - DNA, 26 bp, synthetic construct
  source 1..26: mol_type = other DNA
  misc_feature 1..26: note = Vanillin aptamer
caggagaaacatggagtctcgatgat

SEQ ID NO: 52 - DNA, 10 bp, synthetic construct
  source 1..10: mol_type = other DNA
  misc_feature 1..10: note = Quenching cDNA to Vanillin aptamer
tcatcgagac

SEQ ID NO: 53 - DNA, 65 bp, synthetic construct
  source 1..65: mol_type = other DNA
  misc_feature 1..65: note = Zinc ion aptamer
ccatcagttagtcattacgcttacggcggctctatcctaactgatatattgtgaagtcgtgtccc

SEQ ID NO: 54 - DNA, 15 bp, synthetic construct
  source 1..15: mol_type = other DNA
  misc_feature 1..15: note = Quenching cDNA to Zinc ion aptamer
atgactaactgatgg

SEQ ID NO: 55 - DNA, 27 bp, synthetic construct
  source 1..27: mol_type = other DNA
  misc_feature 1..27: note = IFN gamma aptamer
tggggttggttgtgttgggtgttgtgt

SEQ ID NO: 56 - DNA, 13 bp, synthetic construct
  source 1..13: mol_type = other DNA
  misc_feature 1..13: note = Quenching cDNA to IFN gamma aptamer
acaaccaacccca

SEQ ID NO: 57 - DNA, 34 bp, synthetic construct
  source 1..34: mol_type = other DNA
  misc_feature 1..34: note = gHSA aptamer
ggtgcggttcgtgcggttgtagtactcgtggccg

SEQ ID NO: 58 - DNA, 28 bp, synthetic construct
  source 1..28: mol_type = other DNA
  misc_feature 1..28: note = Negative control DNA
ctcattcaataccctacgtctaccctac

SEQ ID NO: 59 - DNA, 12 bp, synthetic construct
  source 1..12: mol_type = other DNA
  misc_feature 1..12: note = Quenching cDNA to Negative control DNA
gtagggtagacg

SEQ ID NO: 60 - DNA, 831 bp, Saccharomyces cerevisiae
  source 1..831: mol_type = genomic DNA
atgagcaaatacgaaggacgttggacgactgttaaagtcgaattagaggccggtatcgcgtgggtaacgttgaacagacctgagaagaggaatgccatgagccccacgcttaacagggagatggtagatgtgttggagactctggagcaggacgcagacgctggggtattggttcttactggggcgggggaaagttggactgccggcatggacttgaaggaatacttcagggaagtggatgcgggcccagaaatcttgcaggaaaaaaaccgtagggaggcaagccaatggcagtggaagctactaaggttatacgccaagccgactatcgctatggtcaacggttggtgcttcggaggcgggttttcccccctagtcgcatgtgatctagctatatgcgcaaacgaagctacgtttggattaagcgagattaactggggtattcctcctgggaaccttgtcagcaaagcgatggcggatacagtaggccacaggcagtctttgtgttacataatgacagggaaaacttttgatgggaggaaagctgcggagatgggactggtaaatgattccgtcccactggctgagttaagagagactacgagagaactagctttgaaccttctggagaaaaacttggttgtcttgcgtgcggctaaaaacggattcaagaggtgccgtgagctgacgtgggaacagaacgaggactacttatacgcaaagttggaccagtctcgtctacttgacacgaccggagggagggagcaaggcatgaaacaatttttggacgataaaagtattaaacctggattgcaggcttacaagaggtga

SEQ ID NO: 61 - DNA, 831 bp, synthetic construct; identical to SEQ ID NO: 60
  source 1..831: mol_type = other DNA
  misc_feature 1..831: note = Mutant ech gene with I90N/Y169C/N212Q/P213L amino acid substitutions

SEQ ID NO: 62 - DNA, 4146 bp, synthetic construct
  source 1..4146: mol_type = other DNA
  misc_feature 1..4146: note = E. coli S. cerevisiae shuttle vector with AmpR and LEU2 markers
caactttgtatagaaaagttgacccagctttcttgtacaaagtggtagcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctagcggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccggggatcgatccactagt

SEQ ID NO: 63 - DNA, 11240 bp, synthetic construct
  source 1..11240: mol_type = other DNA
  misc_feature 1..11240: note = E. coli S. cerevisiae shuttle vector with AmpR and URA3 markers, expressing Cas9 and containing sgRNA expression cassette
atggacaagaagtattctatcggactggacatcgggactaatagcgtcgggtgggccgtcatcactgacgagtacaaggtgccctctaagaagttcaaggtgctcgggaacaccgaccggcattccatcaagaaaaatctgatcggagctctcctctttgattcaggggagaccgctgaagcaacccgcctcaagcggactgctagacggcggtacaccaggaggaagaaccggatttgttaccttcaagagatattctccaacgaaatggcaaaggtcgacgacagcttcttccataggctggaagaatcattcctcgtggaagaggataagaagcatgaacggcatcccatcttcggtaatatcgtcgacgaggtggcctatcacgagaaatacccaaccatctaccatcttcgcaaaaagctggtggactcaaccgacaaggcagacctccggcttatctacctggccctggcccacatgattaagttcagaggccacttcctgatcgagggcgacctcaatcctgacaatagcgatgtggataaactgttcatccagctggtgcagacttacaaccagctctttgaagagaaccccatcaatgcaagcggagtcgatgccaaggccattctgtcagcccggctgtcaaagagccgcagacttgagaatcttatcgctcagctgccgggtgaaaagaaaaatggactgttcgggaacctgattgctctttcacttgggctgactcccaatttcaagtctaatttcgacctggcagaggatgccaagctgcaactgtccaaggacacctatgatgacgatctcgacaacctcctggcccagatcggtgaccaatacgccgaccttttccttgctgctaagaatctttctgacgccatcctgctgtctgacattctccgcgtgaacactgaaatcaccaaggcccctctttcagcttcaatgattaagcggtatgatgagcaccaccaggacctgaccctgcttaaggcactcgtccggcagcagcttccggagaagtacaaggaaatcttctttgaccagtcaaagaatggatacgccggctacatcgacggaggtgcctcccaagaggaattttataagtttatcaaacctatccttgagaagatggacggcaccgaagagctcctcgtgaaactgaatcgggaggatctgctgcggaagcagcgcactttcgacaatgggagcattccccaccagatccatcttggggagcttcacgccatccttcggcgccaagaggacttctacccctttcttaaggacaacagggagaagattgagaaaattctcactttccgcatcccctactacgtgggacccctcgccagaggaaatagccggtttgcttggatgaccagaaagtcagaagaaactatcactccctggaacttcgaagaggtggtggacaagggagccagcgctcagtcattcatcgaacggatgactaacttcgataagaacctccccaatgagaaggtcctgccgaaacattccctgctctacgagtactttaccgtgtacaacgagctgaccaaggtgaaatatgtcaccgaagggatgaggaagcccgcattcctgtcaggcgaacaaaagaaggcaattgtggaccttctgttcaagaccaatagaaaggtgaccgtgaagcagctgaaggaggactatttcaagaaaattgaatgcttcgactctgtggagattagcggggtcgaagatcggttcaacgcaagcctgggtacctaccatgatctgcttaagatcatcaaggacaaggattttctggacaatgaggagaaagaggacatccttgaggacattgtcctgactctcactctgttcgaggaccgggaaatgatcgaggagaggcttaagacctacgcccatctgttcgacgataaagtgatgaagcaacttaaacggagaagatataccggatggggacgccttagccgcaaactcatcaacggaatccgggacaaacagagcggaaagaccattcttgatttccttaagagcgacggattcgctaatcgcaacttcatgcaacttatccatgatgattccctgacctttaaggaggacatccagaaggcccaagtgtctggacaaggtgactcactgcacgagcatatcgcaaatctggctggttcacccgctattaagaagggtattctccagaccgtgaaagtcgtggacgagctggtcaaggtgatgggtcgccataaaccagagaacattgtcatcgagatggccagggaaaaccagactacccagaagggacagaagaacagcagggagcggatgaaaagaattgaggaagggattaaggagctcgggtcacagatccttaaagagcacccggtggaaaacacccagcttcagaatgagaagctctatctgtactaccttcaaaatggacgcgatatgtatgtggaccaagagcttgatatcaacaggctctcagactacgacgtggaccacatcgtccctcagagcttcctcaaagacgactcaattgacaataaggtgctgactcgctcagacaagaaccggggaaagtcagataacgtgccctcagaggaagtcgtgaaaaagatgaagaactattggcgccagcttctgaacgcaaagctaatcactcagcggaagttcgacaatctcactaaggctgagaggggcggactgagcgaactggacaaagcaggattcattaaacggcaacttgtggagactcggcagattactaaacatgtagcccaaatccttgactcacgcatgaataccaagtacgacgaaaacgacaaacttatccgcgaggtgaaggtgattaccctgaagtccaagctggtcagcgatttcagaaaggactttcaattctacaaagtgcgggagatcaataactatcatcatgctcatgacgcatatctgaatgccgtggtgggaaccgccctaatcaagaagtacccaaagctggaaagcgagttcgtgtacggagactacaaggtctacgacgtgcgcaagatgattgccaaatctgagcaggagatcggaaaggccaccgcaaagtacttcttctacagcaacatcatgaatttcttcaagaccgaaatcacccttgcaaacggtgagatccggaagaggccgctcatcgagactaatggggagactggcgaaatcgtgtgggacaagggcagagatttcgctaccgtgcgcaaagtgctttctatgcctcaagtgaacatcgtgaagaaaaccgaggtgcaaaccggaggcttttctaaggaatcaatcctccccaagcgcaactccgacaagctcattgcaaggaagaaggattgggaccctaagaagtacggcggattcgattcaccaactgtggcttattctgtcctggtcgtggctaaggtggaaaaaggaaagtctaagaagctcaagagcgtgaaggaactgctgggtatcaccattatggagcgcagctccttcgagaagaacccaattgactttctcgaagccaaaggttacaaggaagtcaagaaggaccttatcatcaagctcccaaagtatagcctgttcgaactggagaatgggcggaagcggatgctcgcctccgctggcgaacttcagaagggtaatgagctggctctcccctccaagtacgtgaatttcctctaccttgcaagccattacgagaagctgaaggggagccccgaggacaacgagcaaaagcaactgtttgtggagcagcataagcattatctggacgagatcattgagcagatttccgagttttctaaacgcgtcattctcgctgatgccaacctcgataaagtccttagcgcatacaataagcacagagacaaaccaattcgggagcaggctgagaatatcatccacctgttcaccctcaccaatcttggtgcccctgccgcattcaagtacttcgacaccaccatcgaccggaaacgctatacctccaccaaagaagtgctggacgccaccctcatccaccagagcatcaccggactttacgaaactcggattgacctctcacagctcggaggggatgagggagctcccaagaaaaagcgcaaggtaggtagttccggatcctagactctcgaggcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttctctagagcggccgtggtatcgtttagattggcaattacagtgtcttagctcacatgcttataactaattacatgactcgaagacataaaaaacaaaaaaagcaccaccgactcggtgccactttttcaagttgataacggactagccttattttaacttgctatttaaatgcatcgattctgatcatttatctttcactgcggagaagtttcgaacgccgaaacatgcgcaccaactttcacttctacagcgtttgaccaaaatcttttgaacagaacattgtagggtgtgaaaaaatgcgcacctttaccgctagcccaagagggcactacaaaatctagagttgtacttcaaacgtacatgtaatcaccttgtatatactcgaaagaaaacatcaagtttctgtataaatatgagtgaaagcataatcatacattatcttttcaaagattgtccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcagtctactagtcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaagatctgatatggatcgaattagatctcgccacc

SEQ ID NO: 64 - DNA, 5442 bp, synthetic construct
  source 1..5442: mol_type = other DNA
  misc_feature 1..5442: note = Vector for expression of recombinant protein in E. coli, harboring AmpR marker
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatcccgacccatttgctgtccaccagtcatgctagccatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 65 - DNA, 11240 bp, synthetic construct
  source 1..11240: mol_type = other DNA
  misc_structure 1..11240: note = pML104 derivate, containing the sgRNA for adh6 gene deletion
atggacaagaagtattctatcggactggacatcgggactaatagcgtcgggtgggccgtcatcactgacgagtacaaggtgccctctaagaagttcaaggtgctcgggaacaccgaccggcattccatcaagaaaaatctgatcggagctctcctctttgattcaggggagaccgctgaagcaacccgcctcaagcggactgctagacggcggtacaccaggaggaagaaccggatttgttaccttcaagagatattctccaacgaaatggcaaaggtcgacgacagcttcttccataggctggaagaatcattcctcgtggaagaggataagaagcatgaacggcatcccatcttcggtaatatcgtcgacgaggtggcctatcacgagaaatacccaaccatctaccatcttcgcaaaaagctggtggactcaaccgacaaggcagacctccggcttatctacctggccctggcccacatgattaagttcagaggccacttcctgatcgagggcgacctcaatcctgacaatagcgatgtggataaactgttcatccagctggtgcagacttacaaccagctctttgaagagaaccccatcaatgcaagcggagtcgatgccaaggccattctgtcagcccggctgtcaaagagccgcagacttgagaatcttatcgctcagctgccgggtgaaaagaaaaatggactgttcgggaacctgattgctctttcacttgggctgactcccaatttcaagtctaatttcgacctggcagaggatgccaagctgcaactgtccaaggacacctatgatgacgatctcgacaacctcctggcccagatcggtgaccaatacgccgaccttttccttgctgctaagaatctttctgacgccatcctgctgtctgacattctccgcgtgaacactgaaatcaccaaggcccctctttcagcttcaatgattaagcggtatgatgagcaccaccaggacctgaccctgcttaaggcactcgtccggcagcagcttccggagaagtacaaggaaatcttctttgaccagtcaaagaatggatacgccggctacatcgacggaggtgcctcccaagaggaattttataagtttatcaaacctatccttgagaagatggacggcaccgaagagctcctcgtgaaactgaatcgggaggatctgctgcggaagcagcgcactttcgacaatgggagcattccccaccagatccatcttggggagcttcacgccatccttcggcgccaagaggacttctacccctttcttaaggacaacagggagaagattgagaaaattctcactttccgcatcccctactacgtgggacccctcgccagaggaaatagccggtttgcttggatgaccagaaagtcagaagaaactatcactccctggaacttcgaagaggtggtggacaagggagccagcgctcagtcattcatcgaacggatgactaacttcgataagaacctccccaatgagaaggtcctgccgaaacattccctgctctacgagtactttaccgtgtacaacgagctgaccaaggtgaaatatgtcaccgaagggatgaggaagcccgcattcctgtcaggcgaacaaaagaaggcaattgtggaccttctgttcaagaccaatagaaaggtgaccgtgaagcagctgaaggaggactatttcaagaaaattgaatgcttcgactctgtggagattagcggggtcgaagatcggttcaacgcaagcctgggtacctaccatgatctgcttaagatcatcaaggacaaggattttctggacaatgaggagaaagaggacatccttgaggacattgtcctgactctcactctgttcgaggaccgggaaatgatcgaggagaggcttaagacctacgcccatctgttcgacgataaagtgatgaagcaacttaaacggagaagatataccggatggggacgccttagccgcaaactcatcaacggaatccgggacaaacagagcggaaagaccattcttgatttccttaagagcgacggattcgctaatcgcaacttcatgcaacttatccatgatgattccctgacctttaaggaggacatccagaaggcccaagtgtctggacaaggtgactcactgcacgagcatatcgcaaatctggctggttcacccgctattaagaagggtattctccagaccgtgaaagtcgtggacgagctggtcaaggtgatgggtcgccataaaccagagaacattgtcatcgagatggccagggaaaaccagactacccagaagggacagaagaacagcagggagcggatgaaaagaattgaggaagggattaaggagctcgggtcacagatccttaaagagcacccggtggaaaacacccagcttcagaatgagaagctctatctgtactaccttcaaaatggacgcgatatgtatgtggaccaagagcttgatatcaacaggctctcagactacgacgtggaccacatcgtccctcagagcttcctcaaagacgactcaattgacaataaggtgctgactcgctcagacaagaaccggggaaagtcagataacgtgccctcagaggaagtcgtgaaaaagatgaagaactattggcgccagcttctgaacgcaaagctaatcactcagcggaagttcgacaatctcactaaggctgagaggggcggactgagcgaactggacaaagcaggattcattaaacggcaacttgtggagactcggcagattactaaacatgtagcccaaatccttgactcacgcatgaataccaagtacgacgaaaacgacaaacttatccgcgaggtgaaggtgattaccctgaagtccaagctggtcagcgatttcagaaaggactttcaattctacaaagtgcgggagatcaataactatcatcatgctcatgacgcatatctgaatgccgtggtgggaaccgccctaatcaagaagtacccaaagctggaaagcgagttcgtgtacggagactacaaggtctacgacgtgcgcaagatgattgccaaatctgagcaggagatcggaaaggccaccgcaaagtacttcttctacagcaacatcatgaatttcttcaagaccgaaatcacccttgcaaacggtgagatccggaagaggccgctcatcgagactaatggggagactggcgaaatcgtgtgggacaagggcagagatttcgctaccgtgcgcaaagtgctttctatgcctcaagtgaacatcgtgaagaaaaccgaggtgcaaaccggaggcttttctaaggaatcaatcctccccaagcgcaactccgacaagctcattgcaaggaagaaggattgggaccctaagaagtacggcggattcgattcaccaactgtggcttattctgtcctggtcgtggctaaggtggaaaaaggaaagtctaagaagctcaagagcgtgaaggaactgctgggtatcaccattatggagcgcagctccttcgagaagaacccaattgactttctcgaagccaaaggttacaaggaagtcaagaaggaccttatcatcaagctcccaaagtatagcctgttcgaactggagaatgggcggaagcggatgctcgcctccgctggcgaacttcagaagggtaatgagctggctctcccctccaagtacgtgaatttcctctaccttgcaagccattacgagaagctgaaggggagccccgaggacaacgagcaaaagcaactgtttgtggagcagcataagcattatctggacgagatcattgagcagatttccgagttttctaaacgcgtcattctcgctgatgccaacctcgataaagtccttagcgcatacaataagcacagagacaaaccaattcgggagcaggctgagaatatcatccacctgttcaccctcaccaatcttggtgcccctgccgcattcaagtacttcgacaccaccatcgaccggaaacgctatacctccaccaaagaagtgctggacgccaccctcatccaccagagcatcaccggactttacgaaactcggattgacctctcacagctcggaggggatgagggagctcccaagaaaaagcgcaaggtaggtagttccggatcctagactctcgaggcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttctctagagcggccgtggtatcgtttagattggcaattacagtgtcttagctcacatgcttataactaattacatgactcgaagacataaaaaacaaaaaaagcaccaccgactcggtgccactttttcaagttgataacggacacatgcttcgatcttaatgtctttaaatgcatcgattctgatcatttatctttcactgcggagaagtttcgaacgccgaaacatgcgcaccaactttcacttctacagcgtttgaccaaaatcttttgaacagaacattgtagggtgtgaaaaaatgcgcacctttaccgctagcccaagagggcactacaaaatctagagttgtacttcaaacgtacatgtaatcaccttgtatatactcgaaagaaaacatcaagtttctgtataaatatgagtgaaagcataatcatacattatcttttcaaagattgtccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcagtctactagtcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaagatctgatatggatcgaattagatctcgccacc

SEQ ID NO: 66 - DNA, 11239 bp, synthetic construct
  source 1..11239: mol_type = other DNA
  misc_feature 1..11239: note = pML104 derivate, containing the sgRNA for gre2 gene deletion
atggacaagaagtattctatcggactggacatcgggactaatagcgtcgggtgggccgtcatcactgacgagtacaaggtgccctctaagaagttcaaggtgctcgggaacaccgaccggcattccatcaagaaaaatctgatcggagctctcctctttgattcaggggagaccgctgaagcaacccgcctcaagcggactgctagacggcggtacaccaggaggaagaaccggatttgttaccttcaagagatattctccaacgaaatggcaaaggtcgacgacagcttcttccataggctggaagaatcattcctcgtggaagaggataagaagcatgaacggcatcccatcttcggtaatatcgtcgacgaggtggcctatcacgagaaatacccaaccatctaccatcttcgcaaaaagctggtggactcaaccgacaaggcagacctccggcttatctacctggccctggcccacatgattaagttcagaggccacttcctgatcgagggcgacctcaatcctgacaatagcgatgtggataaactgttcatccagctggtgcagacttacaaccagctctttgaagagaaccccatcaatgcaagcggagtcgatgccaaggccattctgtcagcccggctgtcaaagagccgcagacttgagaatcttatcgctcagctgccgggtgaaaagaaaaatggactgttcgggaacctgattgctctttcacttgggctgactcccaatttcaagtctaatttcgacctggcagaggatgccaagctgcaactgtccaaggacacctatgatgacgatctcgacaacctcctggcccagatcggtgaccaatacgccgaccttttccttgctgctaagaatctttctgacgccatcctgctgtctgacattctccgcgtgaacactgaaatcaccaaggcccctctttcagcttcaatgattaagcggtatgatgagcaccaccaggacctgaccctgcttaaggcactcgtccggcagcagcttccggagaagtacaaggaaatcttctttgaccagtcaaagaatggatacgccggctacatcgacggaggtgcctcccaagaggaattttataagtttatcaaacctatccttgagaagatggacggcaccgaagagctcctcgtgaaactgaatcgggaggatctgctgcggaagcagcgcactttcgacaatgggagcattccccaccagatccatcttggggagcttcacgccatccttcggcgccaagaggacttctacccctttcttaaggacaacagggagaagattgagaaaattctcactttccgcatcccctactacgtgggacccctcgccagaggaaatagccggtttgcttggatgaccagaaagtcagaagaaactatcactccctggaacttcgaagaggtggtggacaagggagccagcgctcagtcattcatcgaacggatgactaacttcgataagaacctccccaatgagaaggtcctgccgaaacattccctgctctacgagtactttaccgtgtacaacgagctgaccaaggtgaaatatgtcaccgaagggatgaggaagcccgcattcctgtcaggcgaacaaaagaaggcaattgtggaccttctgttcaagaccaatagaaaggtgaccgtgaagcagctgaaggaggactatttcaagaaaattgaatgcttcgactctgtggagattagcggggtcgaagatcggttcaacgcaagcctgggtacctaccatgatctgcttaagatcatcaaggacaaggattttctggacaatgaggagaaagaggacatccttgaggacattgtcctgactctcactctgttcgaggaccgggaaatgatcgaggagaggcttaagacctacgcccatctgttcgacgataaagtgatgaagcaacttaaacggagaagatataccggatggggacgccttagccgcaaactcatcaacggaatccgggacaaacagagcggaaagaccattcttgatttccttaagagcgacggattcgctaatcgcaacttcatgcaacttatccatgatgattccctgacctttaaggaggacatccagaaggcccaagtgtctggacaaggtgactcactgcacgagcatatcgcaaatctggctggttcacccgctattaagaagggtattctccagaccgtgaaagtcgtggacgagctggtcaaggtgatgggtcgccataaaccagagaacattgtcatcgagatggccagggaaaaccagactacccagaagggacagaagaacagcagggagcggatgaaaagaattgaggaagggattaaggagctcgggtcacagatccttaaagagcacccggtggaaaacacccagcttcagaatgagaagctctatctgtactaccttcaaaatggacgcgatatgtatgtggaccaagagcttgatatcaacaggctctcagactacgacgtggaccacatcgtccctcagagcttcctcaaagacgactcaattgacaataaggtgctgactcgctcagacaagaaccggggaaagtcagataacgtgccctcagaggaagtcgtgaaaaagatgaagaactattggcgccagcttctgaacgcaaagctaatcactcagcggaagttcgacaatctcactaaggctgagaggggcggactgagcgaactggacaaagcaggattcattaaacggcaacttgtggagactcggcagattactaaacatgtagcccaaatccttgactcacgcatgaataccaagtacgacgaaaacgacaaacttatccgcgaggtgaaggtgattaccctgaagtccaagctggtcagcgatttcagaaaggactttcaattctacaaagtgcgggagatcaataactatcatcatgctcatgacgcatatctgaatgccgtggtgggaaccgccctaatcaagaagtacccaaagctggaaagcgagttcgtgtacggagactacaaggtctacgacgtgcgcaagatgattgccaaatctgagcaggagatcggaaaggccaccgcaaagtacttcttctacagcaacatcatgaatttcttcaagaccgaaatcacccttgcaaacggtgagatccggaagaggccgctcatcgagactaatggggagactggcgaaatcgtgtgggacaagggcagagatttcgctaccgtgcgcaaagtgctttctatgcctcaagtgaacatcgtgaagaaaaccgaggtgcaaaccggaggcttttctaaggaatcaatcctccccaagcgcaactccgacaagctcattgcaaggaagaaggattgggaccctaagaagtacggcggattcgattcaccaactgtggcttattctgtcctggtcgtggctaaggtggaaaaaggaaagtctaagaagctcaagagcgtgaaggaactgctgggtatcaccattatggagcgcagctccttcgagaagaacccaattgactttctcgaagccaaaggttacaaggaagtcaagaaggaccttatcatcaagctcccaaagtatagcctgttcgaactggagaatgggcggaagcggatgctcgcctccgctggcgaacttcagaagggtaatgagctggctctcccctccaagtacgtgaatttcctctaccttgcaagccattacgagaagctgaaggggagccccgaggacaacgagcaaaagcaactgtttgtggagcagcataagcattatctggacgagatcattgagcagatttccgagttttctaaacgcgtcattctcgctgatgccaacctcgataaagtccttagcgcatacaataagcacagagacaaaccaattcgggagcaggctgagaatatcatccacctgttcaccctcaccaatcttggtgcccctgccgcattcaagtacttcgacaccaccatcgaccggaaacgctatacctccaccaaagaagtgctggacgccaccctcatccaccagagcatcaccggactttacgaaactcggattgacctctcacagctcggaggggatgagggagctcccaagaaaaagcgcaaggtaggtagttccggatcctagactctcgaggcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttctctagagcggccgtggtatcgtttagattggcaattacagtgtcttagctcacatgcttataactaattacatgactcgaagacataaaaaacaaaaaaagcaccaccgactcggtgccactttttcaagttgataacggacccgttaaattctcggccttttttaaatgcatcgattctgatcatttatctttcactgcggagaagtttcgaacgccgaaacatgcgcaccaactttcacttctacagcgtttgaccaaaatcttttgaacagaacattgtagggtgtgaaaaaatgcgcacctttaccgctagcccaagagggcactacaaaatctagagttgtacttcaaacgtacatgtaatcaccttgtatatactcgaaagaaaacatcaagtttctgtataaatatgagtgaaagcataatcatacattatcttttcaaagattgtccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcagtctactagtcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaagatctgatatggatcgaattagatctcgccacc

SEQ ID NO: 67 - DNA, 8764 bp, synthetic construct
  source 1..8764: mol_type = other DNA
  misc_feature 1..8764: note = pYCP derivate, containing the expression cassettes for 4cl and ech genes
caactttgtatagaaaagttgaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatgggtgactgtgtcgcacccaaggaggatttaatctttcgttctaagttacccgatatttatatccccaaacacttgccactacatacatattgctttgagaacataagtaaagttggcgacaaatcttgtctaataaacggagctacgggggaaacctttacctactcccaggtcgaattgttgtccaggaaggtagcgtctgggcttaataaactaggaatacagcagggagatacaattatgttattactaccaaattctcctgaatactttttcgcctttctgggcgcctcatatcgtggggcaataagtaccatggctaaccccttctttacaagcgcagaagtaattaagcagctaaaggcgagccaggctaaactgattataactcaagcgtgttacgtcgataaggtaaaggactatgcagcggaaaagaacattcaaattatttgcatagatgacgctccgcaggactgcctacacttctctaaattaatggaggcagatgagagtgagatgcctgaagttgttataaattcagatgacgttgtcgccctgccttactcctcagggacaactggactgccaaaaggagtgatgctgacgcataaaggactggtcacttctgtggcgcagcaagttgatggggacaacccgaatctgtatatgcacagcgaagacgtgatgatatgcatccttccactatttcatatctatagtctaaatgcggtactatgctgtgggctgagagctggggtaacaatacttattatgcaaaagtttgatatcgtaccatttttagaacttattcagaaatataaagtgaccattggacctttcgttcccccgattgtcctggctatagccaagtcaccggtagttgataaatatgatttatcaagtgttagaacggtcatgtccggcgcagcgcctcttggtaaagagcttgaagacgctgtccgtgcgaaattcccgaacgcaaaacttgggcaaggttacggtatgactgaagctgggcctgtccttgcgatgtgtctagcctttgcaaaggagccgtatgagattaagagtggagcgtgcggcactgtggtacgtaatgcggaaatgaagattgtcgaccccgagacaaacgcctccttaccgcgtaaccaaagaggtgaaatctgcattaggggtgatcaaatcatgaagggatatttgaacgaccctgagtcaacaagaacaactatcgacgaggagggctggctacataccggagacattgggtttatcgatgatgatgacgagctatttatagtagatcgtctaaaggaaatcatcaaatacaaggggtttcaggtcgcccctgcagagttagaggcccttctgttgacccatccaacaataagtgacgcagcagtcgtcccgatgattgatgagaaagcaggcgaagtacctgtagcgtttgttgttaggacaaacggtttcacgacaacggaggaggagataaagcaatttgtttcaaagcaggttgtcttctacaagagaattttcagggtcttctttgtggatgccattccaaagtctccgagcgggaaaattcttcgtaaggacctgagagcaaggattgcctctggggaccttccaaaataagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatgagcaaatacgaaggacgttggacgactgttaaagtcgaattagaggccggtatcgcgtgggtaacgttgaacagacctgagaagaggaatgccatgagccccacgcttaacagggagatggtagatgtgttggagactctggagcaggacgcagacgctggggtattggttcttactggggcgggggaaagttggactgccggcatggacttgaaggaatacttcagggaagtggatgcgggcccagaaatcttgcaggaaaaaatccgtagggaggcaagccaatggcagtggaagctactaaggttatacgccaagccgactatcgctatggtcaacggttggtgcttcggaggcgggttttcccccctagtcgcatgtgatctagctatatgcgcaaacgaagctacgtttggattaagcgagattaactggggtattcctcctgggaaccttgtcagcaaagcgatggcggatacagtaggccacaggcagtctttgtattacataatgacagggaaaacttttgatgggaggaaagctgcggagatgggactggtaaatgattccgtcccactggctgagttaagagagactacgagagaactagctttgaaccttctggagaaaaacccggttgtcttgcgtgcggctaaaaacggattcaagaggtgccgtgagctgacgtgggaacagaacgaggactacttatacgcaaagttggaccagtctcgtctacttgacacgaccggagggagggagcaaggcatgaaacaatttttggacgataaaagtattaaacctggattgcaggcttacaagaggtgaacccagctttcttgtacaaagtggtagcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctagcggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccggggatcgatccactagt

SEQ ID NO: 68 - DNA, 8764 bp, synthetic construct
  source 1..8764: mol_type = other DNA
  misc_feature 1..8764: note = pVAN derivate, with mutation site D80V in ech gene
caactttgtatagaaaagttgaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatgggtgactgtgtcgcacccaaggaggatttaatctttcgttctaagttacccgatatttatatccccaaacacttgccactacatacatattgctttgagaacataagtaaagttggcgacaaatcttgtctaataaacggagctacgggggaaacctttacctactcccaggtcgaattgttgtccaggaaggtagcgtctgggcttaataaactaggaatacagcagggagatacaattatgttattactaccaaattctcctgaatactttttcgcctttctgggcgcctcatatcgtggggcaataagtaccatggctaaccccttctttacaagcgcagaagtaattaagcagctaaaggcgagccaggctaaactgattataactcaagcgtgttacgtcgataaggtaaaggactatgcagcggaaaagaacattcaaattatttgcatagatgacgctccgcaggactgcctacacttctctaaattaatggaggcagatgagagtgagatgcctgaagttgttataaattcagatgacgttgtcgccctgccttactcctcagggacaactggactgccaaaaggagtgatgctgacgcataaaggactggtcacttctgtggcgcagcaagttgatggggacaacccgaatctgtatatgcacagcgaagacgtgatgatatgcatccttccactatttcatatctatagtctaaatgcggtactatgctgtgggctgagagctggggtaacaatacttattatgcaaaagtttgatatcgtaccatttttagaacttattcagaaatataaagtgaccattggacctttcgttcccccgattgtcctggctatagccaagtcaccggtagttgataaatatgatttatcaagtgttagaacggtcatgtccggcgcagcgcctcttggtaaagagcttgaagacgctgtccgtgcgaaattcccgaacgcaaaacttgggcaaggttacggtatgactgaagctgggcctgtccttgcgatgtgtctagcctttgcaaaggagccgtatgagattaagagtggagcgtgcggcactgtggtacgtaatgcggaaatgaagattgtcgaccccgagacaaacgcctccttaccgcgtaaccaaagaggtgaaatctgcattaggggtgatcaaatcatgaagggatatttgaacgaccctgagtcaacaagaacaactatcgacgaggagggctggctacataccggagacattgggtttatcgatgatgatgacgagctatttatagtagatcgtctaaaggaaatcatcaaatacaaggggtttcaggtcgcccctgcagagttagaggcccttctgttgacccatccaacaataagtgacgcagcagtcgtcccgatgattgatgagaaagcaggcgaagtacctgtagcgtttgttgttaggacaaacggtttcacgacaacggaggaggagataaagcaatttgtttcaaagcaggttgtcttctacaagagaattttcagggtcttctttgtggatgccattccaaagtctccgagcgggaaaattcttcgtaaggacctgagagcaaggattgcctctggggaccttccaaaataagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatgagcaaatacgaaggacgttggacgactgttaaagtcgaattagaggccggtatcgcgtgggtaacgttgaacagacctgagaagaggaatgccatgagccccacgcttaacagggagatggtagatgtgttggagactctggagcaggacgcagacgctggggtattggttcttactggggcgggggaaagttggactgccggcatggacttgaaggaatacttcagggaagtggttgcgggcccagaaatcttgcaggaaaaaatccgtagggaggcaagccaatggcagtggaagctactaaggttatacgccaagccgactatcgctatggtcaacggttggtgcttcggaggcgggttttcccccctagtcgcatgtgatctagctatatgcgcaaacgaagctacgtttggattaagcgagattaactggggtattcctcctgggaaccttgtcagcaaagcgatggcggatacagtaggccacaggcagtctttgtattacataatgacagggaaaacttttgatgggaggaaagctgcggagatgggactggtaaatgattccgtcccactggctgagttaagagagactacgagagaactagctttgaaccttctggagaaaaacccggttgtcttgcgtgcggctaaaaacggattcaagaggtgccgtgagctgacgtgggaacagaacgaggactacttatacgcaaagttggaccagtctcgtctacttgacacgaccggagggagggagcaaggcatgaaacaatttttggacgataaaagtattaaacctggattgcaggcttacaagaggtgaacccagctttcttgtacaaagtggtagcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctagcggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccggggatcgatccactagt

SEQ ID NO: 69 - DNA, 8764 bp, synthetic construct
  source 1..8764: mol_type = other DNA
  misc_feature 1..8764: note = pVAN derivate, with mutation site Y75H in ech gene
caactttgtatagaaaagttgaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatgggtgactgtgtcgcacccaaggaggatttaatctttcgttctaagttacccgatatttatatccccaaacacttgccactacatacatattgctttgagaacataagtaaagttggcgacaaatcttgtctaataaacggagctacgggggaaacctttacctactcccaggtcgaattgttgtccaggaaggtagcgtctgggcttaataaactaggaatacagcagggagatacaattatgttattactaccaaattctcctgaatactttttcgcctttctgggcgcctcatatcgtggggcaataagtaccatggctaaccccttctttacaagcgcagaagtaattaagcagctaaaggcgagccaggctaaactgattataactcaagcgtgttacgtcgataaggtaaaggactatgcagcggaaaagaacattcaaattatttgcatagatgacgctccgcaggactgcctacacttctctaaattaatggaggcagatgagagtgagatgcctgaagttgttataaattcagatgacgttgtcgccctgccttactcctcagggacaactggactgccaaaaggagtgatgctgacgcataaaggactggtcacttctgtggcgcagcaagttgatggggacaacccgaatctgtatatgcacagcgaagacgtgatgatatgcatccttccactatttcatatctatagtctaaatgcggtactatgctgtgggctgagagctggggtaacaatacttattatgcaaaagtttgatatcgtaccatttttagaacttattcagaaatataaagtgaccattggacctttcgttcccccgattgtcctggctatagccaagtcaccggtagttgataaatatgatttatcaagtgttagaacggtcatgtccggcgcagcgcctcttggtaaagagcttgaagacgctgtccgtgcgaaattcccgaacgcaaaacttgggcaaggttacggtatgactgaagctgggcctgtccttgcgatgtgtctagcctttgcaaaggagccgtatgagattaagagtggagcgtgcggcactgtggtacgtaatgcggaaatgaagattgtcgaccccgagacaaacgcctccttaccgcgtaaccaaagaggtgaaatctgcattaggggtgatcaaatcatgaagggatatttgaacgaccctgagtcaacaagaacaactatcgacgaggagggctggctacataccggagacattgggtttatcgatgatgatgacgagctatttatagtagatcgtctaaaggaaatcatcaaatacaaggggtttcaggtcgcccctgcagagttagaggcccttctgttgacccatccaacaataagtgacgcagcagtcgtcccgatgattgatgagaaagcaggcgaagtacctgtagcgtttgttgttaggacaaacggtttcacgacaacggaggaggagataaagcaatttgtttcaaagcaggttgtcttctacaagagaattttcagggtcttctttgtggatgccattccaaagtctccgagcgggaaaattcttcgtaaggacctgagagcaaggattgcctctggggaccttccaaaataagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatgagcaaatacgaaggacgttggacgactgttaaagtcgaattagaggccggtatcgcgtgggtaacgttgaacagacctgagaagaggaatgccatgagccccacgcttaacagggagatggtagatgtgttggagactctggagcaggacgcagacgctggggtattggttcttactggggcgggggaaagttggactgccggcatggacttgaaggaacacttcagggaagtggatgcgggcccagaaatcttgcaggaaaaaatccgtagggaggcaagccaatggcagtggaagctactaaggttatacgccaagccgactatcgctatggtcaacggttggtgcttcggaggcgggttttcccccctagtcgcatgtgatctagctatatgcgcaaacgaagctacgtttggattaagcgagattaactggggtattcctcctgggaaccttgtcagcaaagcgatggcggatacagtaggccacaggcagtctttgtattacataatgacagggaaaacttttgatgggaggaaagctgcggagatgggactggtaaatgattccgtcccactggctgagttaagagagactacgagagaactagctttgaaccttctggagaaaaacccggttgtcttgcgtgcggctaaaaacggattcaagaggtgccgtgagctgacgtgggaacagaacgaggactacttatacgcaaagttggaccagtctcgtctacttgacacgaccggagggagggagcaaggcatgaaacaatttttggacgataaaagtattaaacctggattgcaggcttacaagaggtgaacccagctttcttgtacaaagtggtagcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctagcggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccggggatcgatccactagt

SEQ ID NO: 70 - DNA, 8764 bp, synthetic construct
  source 1..8764: mol_type = other DNA
  misc_feature 1..8764: note = pVAN derivate, with mutation sites I90N/Y169C/N212Q/P213L in ech gene
caactttgtatagaaaagttgaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatgggtgactgtgtcgcacccaaggaggatttaatctttcgttctaagttacccgatatttatatccccaaacacttgccactacatacatattgctttgagaacataagtaaagttggcgacaaatcttgtctaataaacggagctacgggggaaacctttacctactcccaggtcgaattgttgtccaggaaggtagcgtctgggcttaataaactaggaatacagcagggagatacaattatgttattactaccaaattctcctgaatactttttcgcctttctgggcgcctcatatcgtggggcaataagtaccatggctaaccccttctttacaagcgcagaagtaattaagcagctaaaggcgagccaggctaaactgattataactcaagcgtgttacgtcgataaggtaaaggactatgcagcggaaaagaacattcaaattatttgcatagatgacgctccgcaggactgcctacacttctctaaattaatggaggcagatgagagtgagatgcctgaagttgttataaattcagatgacgttgtcgccctgccttactcctcagggacaactggactgccaaaaggagtgatgctgacgcataaaggactggtcacttctgtggcgcagcaagttgatggggacaacccgaatctgtatatgcacagcgaagacgtgatgatatgcatccttccactatttcatatctatagtctaaatgcggtactatgctgtgggctgagagctggggtaacaatacttattatgcaaaagtttgatatcgtaccatttttagaacttattcagaaatataaagtgaccattggacctttcgttcccccgattgtcctggctatagccaagtcaccggtagttgataaatatgatttatcaagtgttagaacggtcatgtccggcgcagcgcctcttggtaaagagcttgaagacgctgtccgtgcgaaattcccgaacgcaaaacttgggcaaggttacggtatgactgaagctgggcctgtccttgcgatgtgtctagcctttgcaaaggagccgtatgagattaagagtggagcgtgcggcactgtggtacgtaatgcggaaatgaagattgtcgaccccgagacaaacgcctccttaccgcgtaaccaaagaggtgaaatctgcattaggggtgatcaaatcatgaagggatatttgaacgaccctgagtcaacaagaacaactatcgacgaggagggctggctacataccggagacattgggtttatcgatgatgatgacgagctatttatagtagatcgtctaaaggaaatcatcaaatacaaggggtttcaggtcgcccctgcagagttagaggcccttctgttgacccatccaacaataagtgacgcagcagtcgtcccgatgattgatgagaaagcaggcgaagtacctgtagcgtttgttgttaggacaaacggtttcacgacaacggaggaggagataaagcaatttgtttcaaagcaggttgtcttctacaagagaattttcagggtcttctttgtggatgccattccaaagtctccgagcgggaaaattcttcgtaaggacctgagagcaaggattgcctctggggaccttccaaaataagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatgagcaaatacgaaggacgttggacgactgttaaagtcgaattagaggccggtatcgcgtgggtaacgttgaacagacctgagaagaggaatgccatgagccccacgcttaacagggagatggtagatgtgttggagactctggagcaggacgcagacgctggggtattggttcttactggggcgggggaaagttggactgccggcatggacttgaaggaatacttcagggaagtggatgcgggcccagaaatcttgcaggaaaaaaaccgtagggaggcaagccaatggcagtggaagctactaaggttatacgccaagccgactatcgctatggtcaacggttggtgcttcggaggcgggttttcccccctagtcgcatgtgatctagctatatgcgcaaacgaagctacgtttggattaagcgagattaactggggtattcctcctgggaaccttgtcagcaaagcgatggcggatacagtaggccacaggcagtctttgtgttacataatgacagggaaaacttttgatgggaggaaagctgcggagatgggactggtaaatgattccgtcccactggctgagttaagagagactacgagagaactagctttgaaccttctggagaaaaacttggttgtcttgcgtgcggctaaaaacggattcaagaggtgccgtgagctgacgtgggaacagaacgaggactacttatacgcaaagttggaccagtctcgtctacttgacacgaccggagggagggagcaaggcatgaaacaatttttggacgataaaagtattaaacctggattgcaggcttacaagaggtgaacccagctttcttgtacaaagtggtagcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctagcggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccggggatcgatccactagt

SEQ ID NO: 71 - DNA, 6225 bp, synthetic construct
  source 1..6225: mol_type = other DNA
  misc_feature 1..6225: note = pET21b derivate, expressing ech gene
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgtcacctcttgtaagcctgcaatccaggtttaatacttttatcgtccaaaaattgtttcatgccttgctccctccctccggtcgtgtcaagtagacgagactggtccaactttgcgtataagtagtcctcgttctgttcccacgtcagctcacggcacctcttgaatccgtttttagccgcacgcaagacaaccaagtttttctccagaaggttcaaagctagttctctcgtagtctctcttaactcagccagtgggacggaatcatttaccagtcccatctccgcagctttcctcccatcaaaagttttccctgtcattatgtaacacaaagactgcctgtggcctactgtatccgccatcgctttgctgacaaggttcccaggaggaataccccagttaatctcgcttaatccaaacgtagcttcgtttgcgcatatagctagatcacatgcgactaggggggaaaacccgcctccgaagcaccaaccgttgaccatagcgatagtcggcttggcgtataaccttagtagcttccactgccattggcttgcctccctacggtttttttcctgcaagatttctgggcccgcatccacttccctgaagtattccttcaagtccatgccggcagtccaactttcccccgccccagtaagaaccaataccccagcgtctgcgtcctgctccagagtctccaacacatctaccatctccctgttaagcgtggggctcatggcattcctcttctcaggtctgttcaacgttacccacgcgataccggcctctaattcgactttaacagtcgtccaacgtccttcgtatttgctcatacccatttgctgtccaccagtcatgctagccatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 72 - DNA, 6225 bp, synthetic construct
  source 1..6225: mol_type = other DNA
  misc_feature 1..6225: note = pECH derivate, with mutation site Y75H in ech gene
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgtcacctcttgtaagcctgcaatccaggtttaatacttttatcgtccaaaaattgtttcatgccttgctccctccctccggtcgtgtcaagtagacgagactggtccaactttgcgtataagtagtcctcgttctgttcccacgtcagctcacggcacctcttgaatccgtttttagccgcacgcaagacaaccgggtttttctccagaaggttcaaagctagttctctcgtagtctctcttaactcagccagtgggacggaatcatttaccagtcccatctccgcagctttcctcccatcaaaagttttccctgtcattatgtaatacaaagactgcctgtggcctactgtatccgccatcgctttgctgacaaggttcccaggaggaataccccagttaatctcgcttaatccaaacgtagcttcgtttgcgcatatagctagatcacatgcgactaggggggaaaacccgcctccgaagcaccaaccgttgaccatagcgatagtcggcttggcgtataaccttagtagcttccactgccattggcttgcctccctacggattttttcctgcaagatttctgggcccgcatccacttccctgaagtgttccttcaagtccatgccggcagtccaactttcccccgccccagtaagaaccaataccccagcgtctgcgtcctgctccagagtctccaacacatctaccatctccctgttaagcgtggggctcatggcattcctcttctcaggtctgttcaacgttacccacgcgataccggcctctaattcgactttaacagtcgtccaacgtccttcgtatttgctcatacccatttgctgtccaccagtcatgctagccatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 73 - DNA, 6225 bp, synthetic construct
  source 1..6225: mol_type = other DNA
  misc_feature 1..6225: note = pECH derivate, with mutation site D80V in ech gene
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgtcacctcttgtaagcctgcaatccaggtttaatacttttatcgtccaaaaattgtttcatgccttgctccctccctccggtcgtgtcaagtagacgagactggtccaactttgcgtataagtagtcctcgttctgttcccacgtcagctcacggcacctcttgaatccgtttttagccgcacgcaagacaaccgggtttttctccagaaggttcaaagctagttctctcgtagtctctcttaactcagccagtgggacggaatcatttaccagtcccatctccgcagctttcctcccatcaaaagttttccctgtcattatgtaatacaaagactgcctgtggcctactgtatccgccatcgctttgctgacaaggttcccaggaggaataccccagttaatctcgcttaatccaaacgtagcttcgtttgcgcatatagctagatcacatgcgactaggggggaaaacccgcctccgaagcaccaaccgttgaccatagcgatagtcggcttggcgtataaccttagtagcttccactgccattggcttgcctccctacggattttttcctgcaagatttctgggcccgcaaccacttccctgaagtattccttcaagtccatgccggcagtccaactttcccccgccccagtaagaaccaataccccagcgtctgcgtcctgctccagagtctccaacacatctaccatctccctgttaagcgtggggctcatggcattcctcttctcaggtctgttcaacgttacccacgcgataccggcctctaattcgactttaacagtcgtccaacgtccttcgtatttgctcatacccatttgctgtccaccagtcatgctagccatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 74 - DNA, 6225 bp, synthetic construct; identical to SEQ ID NO: 71
  source 1..6225: mol_type = other DNA
  misc_feature 1..6225: note = pECH derivate, with mutation sites I90N/Y169C/N212Q/P213L in ech gene

SEQ ID NO: 75 - DNA, 10454 bp, synthetic construct
  source 1..10454: mol_type = other DNA
  misc_feature 1..10454: note = pVAN derivate, containing the expression cassettes for 4cl, ech and pp_0179 genes
caactttgtatagaaaagttgaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatgggtgactgtgtcgcacccaaggaggatttaatctttcgttctaagttacccgatatttatatccccaaacacttgccactacatacatattgctttgagaacataagtaaagttggcgacaaatcttgtctaataaacggagctacgggggaaacctttacctactcccaggtcgaattgttgtccaggaaggtagcgtctgggcttaataaactaggaatacagcagggagatacaattatgttattactaccaaattctcctgaatactttttcgcctttctgggcgcctcatatcgtggggcaataagtaccatggctaaccccttctttacaagcgcagaagtaattaagcagctaaaggcgagccaggctaaactgattataactcaagcgtgttacgtcgataaggtaaaggactatgcagcggaaaagaacattcaaattatttgcatagatgacgctccgcaggactgcctacacttctctaaattaatggaggcagatgagagtgagatgcctgaagttgttataaattcagatgacgttgtcgccctgccttactcctcagggacaactggactgccaaaaggagtgatgctgacgcataaaggactggtcacttctgtggcgcagcaagttgatggggacaacccgaatctgtatatgcacagcgaagacgtgatgatatgcatccttccactatttcatatctatagtctaaatgcggtactatgctgtgggctgagagctggggtaacaatacttattatgcaaaagtttgatatcgtaccatttttagaacttattcagaaatataaagtgaccattggacctttcgttcccccgattgtcctggctatagccaagtcaccggtagttgataaatatgatttatcaagtgttagaacggtcatgtccggcgcagcgcctcttggtaaagagcttgaagacgctgtccgtgcgaaattcccgaacgcaaaacttgggcaaggttacggtatgactgaagctgggcctgtccttgcgatgtgtctagcctttgcaaaggagccgtatgagattaagagtggagcgtgcggcactgtggtacgtaatgcggaaatgaagattgtcgaccccgagacaaacgcctccttaccgcgtaaccaaagaggtgaaatctgcattaggggtgatcaaatcatgaagggatatttgaacgaccctgagtcaacaagaacaactatcgacgaggagggctggctacataccggagacattgggtttatcgatgatgatgacgagctatttatagtagatcgtctaaaggaaatcatcaaatacaaggggtttcaggtcgcccctgcagagttagaggcccttctgttgacccatccaacaataagtgacgcagcagtcgtcccgatgattgatgagaaagcaggcgaagtacctgtagcgtttgttgttaggacaaacggtttcacgacaacggaggaggagataaagcaatttgtttcaaagcaggttgtcttctacaagagaattttcagggtcttctttgtggatgccattccaaagtctccgagcgggaaaattcttcgtaaggacctgagagcaaggattgcctctggggaccttccaaaataagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatgagcaaatacgaaggacgttggacgactgttaaagtcgaattagaggccggtatcgcgtgggtaacgttgaacagacctgagaagaggaatgccatgagccccacgcttaacagggagatggtagatgtgttggagactctggagcaggacgcagacgctggggtattggttcttactggggcgggggaaagttggactgccggcatggacttgaaggaatacttcagggaagtggatgcgggcccagaaatcttgcaggaaaaaatccgtagggaggcaagccaatggcagtggaagctactaaggttatacgccaagccgactatcgctatggtcaacggttggtgcttcggaggcgggttttcccccctagtcgcatgtgatctagctatatgcgcaaacgaagctacgtttggattaagcgagattaactggggtattcctcctgggaaccttgtcagcaaagcgatggcggatacagtaggccacaggcagtctttgtattacataatgacagggaaaacttttgatgggaggaaagctgcggagatgggactggtaaatgattccgtcccactggctgagttaagagagactacgagagaactagctttgaaccttctggagaaaaacccggttgtcttgcgtgcggctaaaaacggattcaagaggtgccgtgagctgacgtgggaacagaacgaggactacttatacgcaaagttggaccagtctcgtctacttgacacgaccggagggagggagcaaggcatgaaacaatttttggacgataaaagtattaaacctggattgcaggcttacaagaggtgaacccagctttcttgtacaaagtggtagcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatggttcaaagattgactttcagaagacgtttgtcttacaacaccaagtctaaccaaagaagaatcgttagaactccaggtggtagattagtctaccaatacgttaagaagccaaagaaaattccaagatgtggtcaatgtaagtccaagttgagaggtatccaacctgccagaccagctgaaagatccagattgtgttacagaaagaagaccgtcaagagagtttatggtggtgttctatgccacaagtgtgtcaagcaacgtattgtcagagctttcttgattgaagaacaaaagatcgtcaaagttttgaaggctcaacaagcttctgccaaggctggtcgtaaagctgctaagtaagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctagcggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccggggatcgatccactagt

SEQ ID NO: 76 - DNA, 10454 bp, synthetic construct
  source 1..10454: mol_type = other DNA
  misc_feature 1..10454: note = pVAN-PP_0179 derivate, with mutation site G45C in pp_0179 gene
caactttgtatagaaaagttgaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatgggtgactgtgtcgcacccaaggaggatttaatctttcgttctaagttacccgatatttatatccccaaacacttgccactacatacatattgctttgagaacataagtaaagttggcgacaaatcttgtctaataaacggagctacgggggaaacctttacctactcccaggtcgaattgttgtccaggaaggtagcgtctgggcttaataaactaggaatacagcagggagatacaattatgttattactaccaaattctcctgaatactttttcgcctttctgggcgcctcatatcgtggggcaataagtaccatggctaaccccttctttacaagcgcagaagtaattaagcagctaaaggcgagccaggctaaactgattataactcaagcgtgttacgtcgataaggtaaaggactatgcagcggaaaagaacattcaaattatttgcatagatgacgctccgcaggactgcctacacttctctaaattaatggaggcagatgagagtgagatgcctgaagttgttataaattcagatgacgttgtcgccctgccttactcctcagggacaactggactgccaaaaggagtgatgctgacgcataaaggactggtcacttctgtggcgcagcaagttgatggggacaacccgaatctgtatatgcacagcgaagacgtgatgatatgcatccttccactatttcatatctatagtctaaatgcggtactatgctgtgggctgagagctggggtaacaatacttattatgcaaaagtttgatatcgtaccatttttagaacttattcagaaatataaagtgaccattggacctttcgttcccccgattgtcctggctatagccaagtcaccggtagttgataaatatgatttatcaagtgttagaacggtcatgtccggcgcagcgcctcttggtaaagagcttgaagacgctgtccgtgcgaaattcccgaacgcaaaacttgggcaaggttacggtatgactgaagctgggcctgtccttgcgatgtgtctagcctttgcaaaggagccgtatgagattaagagtggagcgtgcggcactgtggtacgtaatgcggaaatgaagattgtcgaccccgagacaaacgcctccttaccgcgtaaccaaagaggtgaaatctgcattaggggtgatcaaatcatgaagggatatttgaacgaccctgagtcaacaagaacaactatcgacgaggagggctggctacataccggagacattgggtttatcgatgatgatgacgagctatttatagtagatcgtctaaaggaaatcatcaaatacaaggggtttcaggtcgcccctgcagagttagaggcccttctgttgacccatccaacaataagtgacgcagcagtcgtcccgatgattgatgagaaagcaggcgaagtacctgtagcgtttgttgttaggacaaacggtttcacgacaacggaggaggagataaagcaatttgtttcaaagcaggttgtcttctacaagagaattttcagggtcttctttgtggatgccattccaaagtctccgagcgggaaaattcttcgtaaggacctgagagcaaggattgcctctggggaccttccaaaataagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatgagcaaatacgaaggacgttggacgactgttaaagtcgaattagaggccggtatcgcgtgggtaacgttgaacagacctgagaagaggaatgccatgagccccacgcttaacagggagatggtagatgtgttggagactctggagcaggacgcagacgctggggtattggttcttactggggcgggggaaagttggactgccggcatggacttgaaggaatacttcagggaagtggatgcgggcccagaaatcttgcaggaaaaaatccgtagggaggcaagccaatggcagtggaagctactaaggttatacgccaagccgactatcgctatggtcaacggttggtgcttcggaggcgggttttcccccctagtcgcatgtgatctagctatatgcgcaaacgaagctacgtttggattaagcgagattaactggggtattcctcctgggaaccttgtcagcaaagcgatggcggatacagtaggccacaggcagtctttgtattacataatgacagggaaaacttttgatgggaggaaagctgcggagatgggactggtaaatgattccgtcccactggctgagttaagagagactacgagagaactagctttgaaccttctggagaaaaacccggttgtcttgcgtgcggctaaaaacggattcaagaggtgccgtgagctgacgtgggaacagaacgaggactacttatacgcaaagttggaccagtctcgtctacttgacacgaccggagggagggagcaaggcatgaaacaatttttggacgataaaagtattaaacctggattgcaggcttacaagaggtgaacccagctttcttgtacaaagtggtagcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatggttcaaagattgactttcagaagacgtttgtcttacaacaccaagtctaaccaaagaagaatcgttagaactccaggtggtagattagtctaccaatacgttaagaagccaaagaaaattccaagatgttgtcaatgtaagtccaagttgagaggtatccaacctgccagaccagctgaaagatccagattgtgttacagaaagaagaccgtcaagagagtttatggtggtgttctatgccacaagtgtgtcaagcaacgtattgtcagagctttcttgattgaagaacaaaagatcgtcaaagttttgaaggctcaacaagcttctgccaaggctggtcgtaaagctgctaagtaagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctagcggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccggggatcgatccactagt

SEQ ID NO: 77 - DNA, 10453 bp, synthetic construct
  source 1..10453: mol_type = other DNA
  misc_feature 1..10453: note = pVAN-PP_0179 derivate, with mutation site K48M in pp_0179 gene
caactttgtatagaaaagttgaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatgggtgactgtgtcgcacccaaggaggatttaatctttcgttctaagttacccgatatttatatccccaaacacttgccactacatacatattgctttgagaacataagtaaagttggcgacaaatcttgtctaataaacggagctacgggggaaacctttacctactcccaggtcgaattgttgtccaggaaggtagcgtctgggcttaataaactaggaatacagcagggagatacaattatgttattactaccaaattctcctgaatactttttcgcctttctgggcgcctcatatcgtggggcaataagtaccatggctaaccccttctttacaagcgcagaagtaattaagcagctaaaggcgagccaggctaaactgattataactcaagcgtgttacgtcgataaggtaaaggactatgcagcggaaaagaacattcaaattatttgcatagatgacgctccgcaggactgcctacacttctctaaattaatggaggcagatgagagtgagatgcctgaagttgttataaattcagatgacgttgtcgccctgccttactcctcagggacaactggactgccaaaaggagtgatgctgacgcataaaggactggtcacttctgtggcgcagcaagttgatggggacaacccgaatctgtatatgcacagcgaagacgtgatgatatgcatccttccactatttcatatctatagtctaaatgcggtactatgctgtgggctgagagctggggtaacaatacttattatgcaaaagtttgatatcgtaccatttttagaacttattcagaaatataaagtgaccattggacctttcgttcccccgattgtcctggctatagccaagtcaccggtagttgataaatatgatttatcaagtgttagaacggtcatgtccggcgcagcgcctcttggtaaagagcttgaagacgctgtccgtgcgaaattcccgaacgcaaaacttgggcaaggttacggtatgactgaagctgggcctgtccttgcgatgtgtctagcctttgcaaaggagccgtatgagattaagagtggagcgtgcggcactgtggtacgtaatgcggaaatgaagattgtcgaccccgagacaaacgcctccttaccgcgtaaccaaagaggtgaaatctgcattaggggtgatcaaatcatgaagggatatttgaacgaccctgagtcaacaagaacaactatcgacgaggagggctggctacataccggagacattgggtttatcgatgatgatgacgagctatttatagtagatcgtctaaaggaaatcatcaaatacaaggggtttcaggtcgcccctgcagagttagaggcccttctgttgacccatccaacaataagtgacgcagcagtcgtcccgatgattgatgagaaagcaggcgaagtacctgtagcgtttgttgttaggacaaacggtttcacgacaacggaggaggagataaagcaatttgtttcaaagcaggttgtcttctacaagagaattttcagggtcttctttgtggatgccattccaaagtctccgagcgggaaaattcttcgtaaggacctgagagcaaggattgcctctggggaccttccaaaataagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatgagcaaatacgaaggacgttggacgactgttaaagtcgaattagaggccggtatcgcgtgggtaacgttgaacagacctgagaagaggaatgccatgagccccacgcttaacagggagatggtagatgtgttggagactctggagcaggacgcagacgctggggtattggttcttactggggcgggggaaagttggactgccggcatggacttgaaggaatacttcagggaagtggatgcgggcccagaaatcttgcaggaaaaaatccgtagggaggcaagccaatggcagtggaagctactaaggttatacgccaagccgactatcgctatggtcaacggttggtgcttcggaggcgggttttcccccctagtcgcatgtgatctagctatatgcgcaaacgaagctacgtttggattaagcgagattaactggggtattcctcctgggaaccttgtcagcaaagcgatggcggatacagtaggccacaggcagtctttgtattacataatgacagggaaaacttttgatgggaggaaagctgcggagatgggactggtaaatgattccgtcccactggctgagttaagagagactacgagagaactagctttgaaccttctggagaaaaacccggttgtcttgcgtgcggctaaaaacggattcaagaggtgccgtgagctgacgtgggaacagaacgaggactacttatacgcaaagttggaccagtctcgtctacttgacacgaccggagggagggagcaaggcatgaaacaatttttggacgataaaagtattaaacctggattgcaggcttacaagaggtgaacccagctttcttgtacaaagtggtagcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatggttcaaagattgactttcagaagacgtttgtcttacaacaccaagtctaaccaaagaagaatcgttagaactccaggtggtagattagtctaccaatacgttaagaagccaaagaaaattccaagatgtggtcaatgtatgtccaagttgagaggtatccaacctgccagaccagctgaaagatccagattgtgttacagaaagaagaccgtcaagagagtttatggtggtgttctatgccacaagtgtgtcaagcaacgtatgtcagagctttcttgattgaagaacaaaagatcgtcaaagttttgaaggctcaacaagcttctgccaaggctggtcgtaaagctgctaagtaagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctagcggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccggggatcgatccactagt

SEQ ID NO: 78 - DNA, 10454 bp, synthetic construct
  source 1..10454: mol_type = other DNA
  misc_feature 1..10454: note = pVAN-PP_0179 derivate, with mutation sites T15I/G45R/Q98H in pp_0179 gene
caactttgtatagaaaagttgaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatgggtgactgtgtcgcacccaaggaggatttaatctttcgttctaagttacccgatatttatatccccaaacacttgccactacatacatattgctttgagaacataagtaaagttggcgacaaatcttgtctaataaacggagctacgggggaaacctttacctactcccaggtcgaattgttgtccaggaaggtagcgtctgggcttaataaactaggaatacagcagggagatacaattatgttattactaccaaattctcctgaatactttttcgcctttctgggcgcctcatatcgtggggcaataagtaccatggctaaccccttctttacaagcgcagaagtaattaagcagctaaaggcgagccaggctaaactgattataactcaagcgtgttacgtcgataaggtaaaggactatgcagcggaaaagaacattcaaattatttgcatagatgacgctccgcaggactgcctacacttctctaaattaatggaggcagatgagagtgagatgcctgaagttgttataaattcagatgacgttgtcgccctgccttactcctcagggacaactggactgccaaaaggagtgatgctgacgcataaaggactggtcacttctgtggcgcagcaagttgatggggacaacccgaatctgtatatgcacagcgaagacgtgatgatatgcatccttccactatttcatatctatagtctaaatgcggtactatgctgtgggctgagagctggggtaacaatacttattatgcaaaagtttgatatcgtaccatttttagaacttattcagaaatataaagtgaccattggacctttcgttcccccgattgtcctggctatagccaagtcaccggtagttgataaatatgatttatcaagtgttagaacggtcatgtccggcgcagcgcctcttggtaaagagcttgaagacgctgtccgtgcgaaattcccgaacgcaaaacttgggcaaggttacggtatgactgaagctgggcctgtccttgcgatgtgtctagcctttgcaaaggagccgtatgagattaagagtggagcgtgcggcactgtggtacgtaatgcggaaatgaagattgtcgaccccgagacaaacgcctccttaccgcgtaaccaaagaggtgaaatctgcattaggggtgatcaaatcatgaagggatatttgaacgaccctgagtcaacaagaacaactatcgacgaggagggctggctacataccggagacattgggtttatcgatgatgatgacgagctatttatagtagatcgtctaaaggaaatcatcaaatacaaggggtttcaggtcgcccctgcagagttagaggcccttctgttgacccatccaacaataagtgacgcagcagtcgtcccgatgattgatgagaaagcaggcgaagtacctgtagcgtttgttgttaggacaaacggtttcacgacaacggaggaggagataaagcaatttgtttcaaagcaggttgtcttctacaagagaattttcagggtcttctttgtggatgccattccaaagtctccgagcgggaaaattcttcgtaaggacctgagagcaaggattgcctctggggaccttccaaaataagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatgagcaaatacgaaggacgttggacgactgttaaagtcgaattagaggccggtatcgcgtgggtaacgttgaacagacctgagaagaggaatgccatgagccccacgcttaacagggagatggtagatgtgttggagactctggagcaggacgcagacgctggggtattggttcttactggggcgggggaaagttggactgccggcatggacttgaaggaatacttcagggaagtggatgcgggcccagaaatcttgcaggaaaaaatccgtagggaggcaagccaatggcagtggaagctactaaggttatacgccaagccgactatcgctatggtcaacggttggtgcttcggaggcgggttttcccccctagtcgcatgtgatctagctatatgcgcaaacgaagctacgtttggattaagcgagattaactggggtattcctcctgggaaccttgtcagcaaagcgatggcggatacagtaggccacaggcagtctttgtattacataatgacagggaaaacttttgatgggaggaaagctgcggagatgggactggtaaatgattccgtcccactggctgagttaagagagactacgagagaactagctttgaaccttctggagaaaaacccggttgtcttgcgtgcggctaaaaacggattcaagaggtgccgtgagctgacgtgggaacagaacgaggactacttatacgcaaagttggaccagtctcgtctacttgacacgaccggagggagggagcaaggcatgaaacaatttttggacgataaaagtattaaacctggattgcaggcttacaagaggtgaacccagctttcttgtacaaagtggtagcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatggttcaaagattgactttcagaagacgtttgtcttacaacatcaagtctaaccaaagaagaatcgttagaactccaggtggtagattagtctaccaatacgttaagaagccaaagaaaattccaagatgtcgtcaatgtaagtccaagttgagaggtatccaacctgccagaccagctgaaagatccagattgtgttacagaaagaagaccgtcaagagagtttatggtggtgttctatgccacaagtgtgtcaagcaacgtattgtcagagctttcttgattgaagaacacaagatcgtcaaagttttgaaggctcaacaagcttctgccaaggctggtcgtaaagctgctaagtaagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctagcggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccggggatcgatccactagt

SEQ ID NO: 79 - DNA, 10454 bp, synthetic construct
  source 1..10454: mol_type = other DNA
  misc_feature 1..10454: note = pVAN-PP_0179 derivate, with mutation sites K40R/V79D in pp_0179 gene
caactttgtatagaaaagttgaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatgggtgactgtgtcgcacccaaggaggatttaatctttcgttctaagttacccgatatttatatccccaaacacttgccactacatacatattgctttgagaacataagtaaagttggcgacaaatcttgtctaataaacggagctacgggggaaacctttacctactcccaggtcgaattgttgtccaggaaggtagcgtctgggcttaataaactaggaatacagcagggagatacaattatgttattactaccaaattctcctgaatactttttcgcctttctgggcgcctcatatcgtggggcaataagtaccatggctaaccccttctttacaagcgcagaagtaattaagcagctaaaggcgagccaggctaaactgattataactcaagcgtgttacgtcgataaggtaaaggactatgcagcggaaaagaacattcaaattatttgcatagatgacgctccgcaggactgcctacacttctctaaattaatggaggcagatgagagtgagatgcctgaagttgttataaattcagatgacgttgtcgccctgccttactcctcagggacaactggactgccaaaaggagtgatgctgacgcataaaggactggtcacttctgtggcgcagcaagttgatggggacaacccgaatctgtatatgcacagcgaagacgtgatgatatgcatccttccactatttcatatctatagtctaaatgcggtactatgctgtgggctgagagctggggtaacaatacttattatgcaaaagtttgatatcgtaccatttttagaacttattcagaaatataaagtgaccattggacctttcgttcccccgattgtcctggctatagccaagtcaccggtagttgataaatatgatttatcaagtgttagaacggtcatgtccggcgcagcgcctcttggtaaagagcttgaagacgctgtccgtgcgaaattcccgaacgcaaaacttgggcaaggttacggtatgactgaagctgggcctgtccttgcgatgtgtctagcctttgcaaaggagccgtatgagattaagagtggagcgtgcggcactgtggtacgtaatgcggaaatgaagattgtcgaccccgagacaaacgcctccttaccgcgtaaccaaagaggtgaaatctgcattaggggtgatcaaatcatgaagggatatttgaacgaccctgagtcaacaagaacaactatcgacgaggagggctggctacataccggagacattgggtttatcgatgatgatgacgagctatttatagtagatcgtctaaaggaaatcatcaaatacaaggggtttcaggtcgcccctgcagagttagaggcccttctgttgacccatccaacaataagtgacgcagcagtcgtcccgatgattgatgagaaagcaggcgaagtacctgtagcgtttgttgttaggacaaacggtttcacgacaacggaggaggagataaagcaatttgtttcaaagcaggttgtcttctacaagagaattttcagggtcttctttgtggatgccattccaaagtctccgagcgggaaaattcttcgtaaggacctgagagcaaggattgcctctggggaccttccaaaataagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatgagcaaatacgaaggacgttggacgactgttaaagtcgaattagaggccggtatcgcgtgggtaacgttgaacagacctgagaagaggaatgccatgagccccacgcttaacagggagatggtagatgtgttggagactctggagcaggacgcagacgctggggtattggttcttactggggcgggggaaagttggactgccggcatggacttgaaggaatacttcagggaagtggatgcgggcccagaaatcttgcaggaaaaaatccgtagggaggcaagccaatggcagtggaagctactaaggttatacgccaagccgactatcgctatggtcaacggttggtgcttcggaggcgggttttcccccctagtcgcatgtgatctagctatatgcgcaaacgaagctacgtttggattaagcgagattaactggggtattcctcctgggaaccttgtcagcaaagcgatggcggatacagtaggccacaggcagtctttgtattacataatgacagggaaaacttttgatgggaggaaagctgcggagatgggactggtaaatgattccgtcccactggctgagttaagagagactacgagagaactagctttgaaccttctggagaaaaacccggttgtcttgcgtgcggctaaaaacggattcaagaggtgccgtgagctgacgtgggaacagaacgaggactacttatacgcaaagttggaccagtctcgtctacttgacacgaccggagggagggagcaaggcatgaaacaatttttggacgataaaagtattaaacctggattgcaggcttacaagaggtgaacccagctttcttgtacaaagtggtagcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaacaagtttgtacaaaaaagcaggctgccaccatggttcaaagattgactttcagaagacgtttgtcttacaacaccaagtctaaccaaagaagaatcgttagaactccaggtggtagattagtctaccaatacgttaagaagccaaagagaattccaagatgtggtcaatgtaagtccaagttgagaggtatccaacctgccagaccagctgaaagatccagattgtgttacagaaagaagaccgtcaagagagtttatggtggtgatctatgccacaagtgtgtcaagcaacgtattgtcagagctttcttgattgaagaacaaaagatcgtcaaagttttgaaggctcaacaagcttctgccaaggctggtcgtaaagctgctaagtaagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatagcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctagcggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccggggatcgatccactagt

SEQ ID NO: 80 - DNA, 28 bp, synthetic construct
  source 1..28: mol_type = other DNA
  misc_feature 1..28: note = ATP aptamer
tacctgggggagtattgcggaggaaggt

SEQ ID NO: 81 - DNA, 51 bp, synthetic construct; identical to SEQ ID NO: 49
  source 1..51: mol_type = other DNA
  misc_feature 1..51: note = glucose aptamer

SEQ ID NO: 82 - DNA, 26 bp, synthetic construct
  source 1..26: mol_type = other DNA
  misc_feature 1..26: note = Vanillin aptamer
caggagaaacatggagtctcgatgat

SEQ ID NO: 83 - DNA, 27 bp, synthetic construct
  source 1..27: mol_type = other DNA
  misc_feature 1..27: note = Naringenin aptamer
ctagcagttgtggtagagtcaccacaa

SEQ ID NO: 84 - DNA, 22 bp, synthetic construct
  source 1..22: mol_type = other DNA
  misc_feature 1..22: note = Arginine aptamer
ccaccaacgtgtcgcctcgtcg

SEQ ID NO: 85 - DNA, 59 bp, synthetic construct
  source 1..59: mol_type = other DNA
  misc_feature 1..59: note = L-tyrosine aptamer
ctctcgggacgacctctcgggacgacggcccgatctcagagtagtcgtcccgtcgtccc

SEQ ID NO: 86 - DNA, 51 bp, synthetic construct
  source 1..51: mol_type = other DNA
  misc_feature 1..51: note = Phenylalanine aptamer
ctctcgggacgaccgcgtttcccaagaaagcaagtattggttggtcgtccc

SEQ ID NO: 87 - DNA, 30 bp, synthetic construct
  source 1..30: mol_type = other DNA
  misc_feature 1..30: note = L-Lactate aptamer
gactagcgcgtatgaatgcttttctatgga

SEQ ID NO: 88 - DNA, 34 bp, synthetic construct
  source 1..34: mol_type = other DNA
  misc_feature 1..34: note = L-tryptophan aptamer
agcacgttggttaggtcaggtttgggtttcgtgc

SEQ ID NO: 89 - DNA, 51 bp, synthetic construct
  source 1..51: mol_type = other DNA
  misc_feature 1..51: note = Testosterone aptamer
ctctcgggacgacgggatgtccggggtacggtggttgcagttcgtcgtccc

SEQ ID NO: 90 - DNA, 51 bp, synthetic construct
  source 1..51: mol_type = other DNA
  misc_feature 1..51: note = Hydrocortisone aptamer
ctctcgggacgacgcccgcatgttccatggatagtcttgactagtcgtccc

SEQ ID NO: 91 - DNA, 51 bp, synthetic construct
  source 1..51: mol_type = other DNA
  misc_feature 1..51: note = Aldosterone aptamer
ctctcgggacgacagatagttgttcttagcgatgttcagcgttgtcgtccc

SEQ ID NO: 92 - DNA, 27 bp, synthetic construct
  source 1..27: mol_type = other DNA
  misc_feature 1..27: note = Human IFN gamma aptamer
tggggttggttgtgttgggtgttgtgt

SEQ ID NO: 93 - DNA, 34 bp, synthetic construct; identical to SEQ ID NO: 57
  source 1..34: mol_type = other DNA
  misc_feature 1..34: note = glycosylated HSA aptamer

SEQ ID NO: 94 - DNA, 25 bp, synthetic construct
  source 1..25: mol_type = other DNA
  misc_feature 1..25: note = Human TNF alpha aptamer
tggtggatggcgcagtcggcgacaa

SEQ ID NO: 95 - DNA, 30 bp, synthetic construct
  source 1..30: mol_type = other DNA
  misc_feature 1..30: note = Human insulin aptamer
ggtggtggggggggttggtagggtgtcttc

SEQ ID NO: 96 - DNA, 29 bp, synthetic construct
  source 1..29: mol_type = other DNA
  misc_feature 1..29: note = Human IL-2 aptamer
tcgttcgggactgctcgggattgcggata

SEQ ID NO: 97 - DNA, 72 bp, synthetic construct
  source 1..72: mol_type = other DNA
  misc_feature 1..72: note = alpha defensin human neutrophil protein 1 aptamer
ggcaggaagacaaacacggggcggccgaaacaatgcgtttcggacccagcaactcgtggtctgtggtgctgt

SEQ ID NO: 98 - DNA, 33 bp, synthetic construct
  source 1..33: mol_type = other DNA
  misc_feature 1..33: note = PD-L1 aptamer
tacaggttctggggggtgggtggggaacctgtt

SEQ ID NO: 99 - DNA, 16 bp, synthetic construct
  source 1..16: mol_type = other DNA
  misc_feature 1..16: note = Thrombin aptamer
cctgccacgctccgct

SEQ ID NO: 100 - DNA, 30 bp, synthetic construct
  source 1..30: mol_type = other DNA
  misc_feature 1..30: note = Salivary alpha amylase aptamer
tgaacgacgtgaatagtgtttgtgggtccg

SEQ ID NO: 101 - DNA, 60 bp, synthetic construct
  source 1..60: mol_type = other DNA
  misc_feature 1..60: note = Streptavidin aptamer
ctatactccactttgctatttctcggttccttcacgcgccgatcgcaggctgatgaattg